(19) United States
(12) Patent Application Publication
(10) Pub. No.: US 2025/0169710 A1
(43) Pub. Date: May 29, 2025

(54) BIOIMPEDANCE-BASED FEEDBACK FOR MEDICAL PROCEDURES

(71) Applicant: EDWARDS LIFESCIENCES CORPORATION, Irvine, CA (US)

(72) Inventors: Bezalel Haberman-Browns, Pardes Hanna (IL); Carmel Peleg, Pardes Hana-Karkur (IL); Lior Harush, Herzliya (IL); Micha Nisani, Hod HaSharon (IL); Yaron Herman, Givat Ada (IL); Natan Anatoly Bukhdruker, Tirat Carmel (IL); Doron Katzir, Sde-Yitzhak (IL); Sergio Delgado, Irvine, CA (US); Yuval Keret, Tel Aviv (IL)

(21) Appl. No.: 19/032,436

(22) Filed: Jan. 20, 2025

Related U.S. Application Data
(63) Continuation of application No. PCT/US2023/028329, filed on Jul. 21, 2023.
(60) Provisional application No. 63/439,836, filed on Jan. 18, 2023.
(60) Provisional application No. 63/369,176, filed on Jul. 22, 2022.

(51) Int. Cl. A61B 5/0538; A61F 2/24

(52) U.S. Cl. CPC A61B 5/0538; A61F 2/246; A61F 2/2466

(57) ABSTRACT

Medical procedures and devices that use bioimpedance-based feedback are disclosed. Bioimpedance-based feedback can include measuring or acquiring electrical signals that include or indicate a bioimpedance signal. The bioimpedance signal can be used to determine the position and/or status of device (e.g., of a clasp or anchor of the device) and/or tissue near the device. The bioimpedance signal can be analyzed and converted into information presented to a clinician to indicate a status of a portion of a device to provide feedback regarding the position and/or status of the device, for example, anchoring elements of an implant. Some devices enable the removal of electrodes or electrical leads when the device is implanted.

### CROSS-REFERENCE TO RELATED APPLICATIONS

This application is a continuation of International Patent Application No. PCT/US2023/028329, filed Jul. 21, 2023, which claims the benefit of U.S. Provisional Application No. 63/369,176, filed Jul. 22, 2022, and to U.S. Provisional Application No. 63/439,836, filed Jan. 18, 2023, the complete disclosures of which are hereby incorporated by reference in their entireties.

### DESCRIPTION OF RELATED ART

Various medical procedures involve implanting an object in the body of a patient to address one or more issues. Medical personnel may use various surgical techniques or other techniques to implant the object in the patient. This may involve securing or anchoring the implant to targeted tissue within the patient.

### SUMMARY

This summary is meant to provide some examples and is not intended to limit the scope of the disclosed subject matter in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

In some implementations, medical procedures, systems, and/or devices that sense or indicate bioimpedance and/or use bioimpedance-based feedback are disclosed. Sensing bioimpedance and/or bioimpedance-based feedback can include measuring or acquiring electrical signals that comprise and/or indicate a bioimpedance signal.

In some implementations, a system, an apparatus, and/or a device can be configured to measure or acquire electrical signals that comprise and/or indicate bioimpedance or a bioimpedance signal. The bioimpedance or bioimpedance signal can be used to obtain/provide information about native anatomy, blood, tissue, cells, vessels, etc. and/or information about various functions, features, locations, uses, operations, etc. of the system, apparatus, and/or device when inside a body of a patient.

In some implementations, the bioimpedance or bioimpedance signal can be used to obtain/provide information about whether the system/device is in the correct location or position, whether the system/device is interacting properly with the native tissue, whether the system/device is properly anchored to the native tissue, whether the system/device is properly implanted, and/or other indications. In some implementations, the bioimpedance signal configured to indicate a leaflet capture status within an anchor (e.g., a capture portion, clasp, clamp, clip, etc.) of the system, apparatus, and/or device.

In some implementations, the system and/or apparatus comprises a device, which can be one or more of a treatment device, a repair device, a valve repair device, an implantable device, a valve treatment device, a tissue treatment device, a catheter, an implant, an anchor, etc.

In some implementations, the system, apparatus, and/or device comprises one or more electrodes. In some implementations, the system, apparatus, and/or device comprises two or more electrodes (or at least two electrodes). In some implementations, the system, apparatus, and/or device comprises three electrodes. In some implementations, the system, apparatus, and/or device comprises four electrodes.

In some implementations, the electrode or electrodes (e.g., two or more electrodes) are configured such than when an electrical signal is applied to the electrode or electrodes, a bioimpedance signal is measured and/or indicated in response to the electrical signal applied.

In some implementations, the system, apparatus, and/or device comprises two or more electrodes including a first electrode coupled to a first location of the system, apparatus, and/or device and a second electrode coupled to a second location of the system, apparatus, and/or device. In some implementations, the first electrode is adjacent to the second electrode. In some implementations, the first electrode comprises an electrode plate covering a majority of the first location and the second electrode comprises an electrode plate covering a majority of the second location.

In some implementations, the system, apparatus, and/or device comprises two or more electrodes including a first electrode coupled to a first arm (e.g., a clip arm, a clasp arm, a paddle, etc.) of the system, apparatus, and/or device and a second electrode coupled to a second portion or second arm (e.g., a second arm, a clip arm, a clasp arm, a paddle, an extension, etc.) of the system, apparatus, and/or device. While many examples herein use a “second arm” for illustrative purposes, other portions (e.g., a coaptation element, a cover, a surface, etc.) of the device can be used as the second portion, even if not typically considered an arm.

In some implementations, the first electrode is adjacent to the second electrode upon closing or moving the first arm and the second arm together.

In some implementations, the first electrode and the second electrode are on the same arm (e.g., both on the first arm or both on the second arm).

In some implementations, a first extension is used as or in place of the first arm and a second extension is used as or in place of the second arm.

In some implementations, a first surface is used as or in place of the first arm and a second surface is used as or in place of the second arm. In some implementations, a first panel is used as or in place of the first arm and a second panel is used as or in place of the second arm.

In some implementations, the first arm and the second arm are arms of a clasp. In some implementations, the first arm and the second arm are arms of a gripper or gripping member.

In some implementations, the first arm and the second arm are arms of a clamp or clamping portion of the system/device.

In some implementations, the first arm and the second arm are arms of an anchor (e.g., clasp, clip, clamp, gripping members, paddles, gripping member and paddle, etc.) of an implant or device (e.g., a treatment device, repair device, etc.).

In some implementations, the first electrode comprises an electrode plate covering a majority of the first arm and the second electrode comprises an electrode plate covering a majority of the second arm.

In some implementations, the two or more electrodes comprise a first electrode coupled to the first arm and a second electrode coupled to the first arm. In some implementations, the first electrode is separated from the second electrode by a gap. In some implementations, the first electrode and the second electrode comprise electrode strips parallel to a length of the first arm. In some implementations, the first electrode and the second electrode comprise electrode strips parallel to a width of the first arm (e.g., perpendicular to a length of the first arm).

In some implementations, the system/device is configured to capture a tissue, e.g., to capture tissue between the first arm and the second arm and/or between a first surface and a second surface, etc. In some implementations, the system/device is configured to capture a leaflet of a native valve, e.g., to capture a leaflet between the first arm and the second arm and/or between a first surface and a second surface, etc.

In some implementations, the first electrode is positioned on the first arm at a first leaflet capture depth, and the second electrode is positioned on the first arm at a second leaflet capture depth. In some implementations, the first electrode is positioned on the first arm at a targeted minimum leaflet capture depth, and the second electrode is positioned on the first arm at a targeted maximum leaflet capture depth.

In some implementations, the first electrode is positioned on the first arm at a first tissue capture depth, and the second electrode is positioned on the first arm at a second tissue capture depth. In some implementations, the first electrode is positioned on the first arm at a targeted minimum tissue capture depth, and the second electrode is positioned on the first arm at a targeted maximum tissue capture depth.

In some implementations, the system/device includes an electrode plate coupled to the second arm.

In some implementations, the system/device includes an impedance measurement device configured to measure bioimpedance or bioimpedance signals and to determine a tissue or leaflet capture depth based on the measured bioimpedance signals.

In some implementations, the system, apparatus, and/or device can be configured for repairing or treating a native valve of a patient or simulation. In some implementations, the system, apparatus, and/or device can be configured for repairing or treating a heart of a patient or simulation.

In some implementations, the system, apparatus, and/or device can comprise an anchor, tissue engagement portion, or clasp (e.g., one, two, three, or more anchors, clasps, or other tissue engagement portion), the anchor or clasp (e.g., each anchor and/or each clasp) comprising a first arm (e.g., a clasp arm, paddle, etc.) and a second arm (e.g., a clasp arm, paddle, etc.).

In some implementations, the first arm and the second arm are joined by a hinge portion to enable the first arm and the second arm to close (e.g., to move toward each other, to come close together, and/or optionally to come into contact with each other) to capture targeted tissue (e.g., a leaflet and/or other tissue) in the anchor, tissue engagement portion, or clasp.

In some implementations, the anchor, tissue engagement portion, or clasp is movable to form a capture region for capturing tissue, e.g., for capturing a leaflet of a native valve. In some implementations, a first arm of the anchor, tissue engagement portion, or clasp is movable toward and away from a second arm (and/or other second portion) of the anchor, tissue engagement portion, or clasp to form a capture region for capturing tissue, e.g., for capturing a leaflet of a native valve.

In some implementations, two or more electrodes are coupled to the anchor or clasp. In some implementations, the two or more electrodes are configured such that when an electrical signal is applied to the two or more electrodes, bioimpedance or a bioimpedance signal can be measured, e.g., based on or in response to the applied electrical signal. In some implementations, the bioimpedance signal configured to indicate a status of the tissue (e.g., a leaflet capture status, etc.) within the anchor or clasp (e.g., between a first arm and a second arm of the anchor or clasp, between a first surface and a second surface of the anchor or clasp, etc.) and/or to indicate a status or deployment status of the anchor or clasp.

In some implementations, the two or more electrodes comprise a first electrode coupled to the first arm and a second electrode coupled to the second arm. In some implementations, the first electrode is adjacent to the second electrode upon closing the anchor or clasp. In some implementations, the first electrode comprises an electrode plate covering a majority of the first arm and the second electrode comprises an electrode plate covering a majority of the second arm.

In some implementations, the two or more electrodes comprise a first electrode coupled to the first arm and a second electrode coupled to the first arm. In some implementations, the first electrode is separated from the second electrode by a gap.

In some implementations, the first electrode and the second electrode comprise electrode strips parallel to a length of the first arm. In some implementations, the first electrode and the second electrode comprise electrode strips parallel to a width of the first arm.

In some implementations, the first electrode is positioned on the first arm at a targeted minimum leaflet capture depth. In some implementations, the second electrode is positioned on the first arm at a targeted maximum leaflet capture depth.

In some implementations, the system, apparatus, and/or device includes an electrode plate coupled to the second arm.

In some implementations, the system, apparatus, and/or device includes an impedance measurement device configured to measure bioimpedance signals and to determine a tissue (e.g., leaflet, etc.) capture depth based on the measured bioimpedance signals.

In some implementations, the impedance measurement device implements an algorithm to generate an indicator of fully captured tissue (e.g., a fully captured leaflet, etc.), partially captured tissue (e.g., a partially captured leaflet, etc.), and/or overly captured tissue (e.g., an overly captured leaflet, etc.).

In some implementations, the impedance measurement device implements an algorithm to generate an indicator of tissue (e.g., leaflet, etc.) capture depth.

In some implementations, the impedance measurement device is configured to generate an indicator of tissue (e.g., leaflet, etc.) capture status when the anchor, tissue engagement portion, or clasp is closed.

In some implementations, the impedance measurement device is configured to generate an indicator of tissue (e.g., leaflet, etc.) capture status when the anchor, tissue engagement portion, or clasp is open.

In some implementations, the impedance measurement device implements an algorithm to generate an indicator of tissue (e.g., leaflet, etc.) capture angle or otherwise provide an indication when captured tissue is askew.

In some implementations, the system/device comprises a device or implant configured to be used and/or implanted during a medical procedure. In some implementations, the device includes an anchor configured to secure the device to tissue in a patient.

In some implementations, the system, apparatus, and/or device comprises an electrode (e.g., at least one electrode, two electrodes, three electrodes, four electrodes, an electrode strip, two electrode strips, three electrode strips, four electrode strips, etc.) coupled to the anchor.

In some implementations, the system, apparatus, and/or device is configured such that, when an electrical signal is applied to the anchor, a bioimpedance signal can be measured based on and/or responsive to the applied electrical signal.

In some implementations, the bioimpedance signal is configured to indicate a position and/or deployment status of the anchor.

In some implementations, the system, apparatus, and/or device comprises an edge-to-edge repair device.

In some implementations, the system, apparatus, and/or device comprises an annuloplasty device, (e.g., an annuloplasty implant, an annuloplasty ring, etc.).

In some implementations, the system apparatus, and/or device includes a plurality of anchors (e.g., two, three, four, or more anchors), each anchor including an electrode. In some implementations, when an electrical signal is applied to the plurality of anchors, a bioimpedance signal can be measured from each of the plurality of anchors based on and/or in response to the electrical signal applied.

In some implementations, each bioimpedance signal is configured to indicate a position and/or deployment status of a corresponding anchor of the plurality of anchors.

In some implementations, the system, apparatus, and/or device includes a plurality of anchors that are electrically shorted together.

In some implementations, the system, apparatus, and/or device includes an impedance measurement device configured to measure bioimpedance signals and to determine a position and/or an anchor deployment status based on the measured bioimpedance signals.

In some implementations, the impedance measurement device implements an algorithm to generate an indicator of anchor deployment status.

In some implementations, the impedance measurement device implements an algorithm to generate an indicator of the anchor deployment status including the anchor in contact with tissue.

In some implementations, the impedance measurement device implements an algorithm to generate an indicator of a partially deployed anchor.

In some implementations, the impedance measurement device implements an algorithm to generate an indicator of a fully deployed anchor.

In some implementations, the system/apparatus/device can comprise a sensor, wherein the sensor is configured to measure impedance or bioimpedance.

In some implementations, the sensor can be configured to compare one or more electrical signals and/or characteristics measured at the time of use to previously measured electrical signals and/or characteristics (e.g., which can correspond to known tissue and blood samples).

In some implementations, the sensor can be configured to determine whether tissue is engaged.

In some implementations, the sensor is configured to differentiate between leaflet tissue, annulus tissue, and/or chordae tendinea tissue (e.g., to differentiate when an anchor is in contact with (or engaged with) leaflet tissue vs. annulus tissue vs. chordae tissue, and/or whether it is only in contact with blood).

In some implementations, a first impedance or bioimpedance value is measured in a method of identifying a position and/or condition of the system/apparatus/device (e.g., of a treatment device, a repair device, an implantable device, of a delivery device, etc.). In some implementations, the first impedance value is compared to a reference value (e.g., to previously measured or determined impedance values, etc.).

In some implementations, the method includes determining and/or estimating one or more of the condition or location of an anchor (e.g., clasp, clip, tissue anchor, helical anchor, dart, screw, etc.) of the system/device based on the comparison.

In some implementations, the method includes determining and/or estimating one or more of the condition or location of a catheter and/or other delivery apparatus of the system/device based on the comparison.

In some implementations, a system, apparatus, and/or device includes a tissue engagement portion or tissue capture portion including a first surface and a second surface, the tissue engagement portion or tissue capture portion configured such that the first surface and the second surface can close or be moved closer together to engage and/or capture tissue in the tissue engagement portion or tissue capture portion.

In some implementations, at least one of the first surface and the second surface are movable to form a capture region between the first surface and the second surface for capturing the tissue.

In some implementations, the tissue engagement portion or tissue capture portion is configured as or to include one or more of the following: an anchor, a dart, a hook, a clasp, a clip, a clamp, a gripper, a gripping member, paddles, arms, combinations of these, etc.

In some implementations, two or more electrodes are coupled to the tissue engagement portion or tissue capture portion. In some implementations, the system, apparatus, and/or device is configured such that: an electrical signal (e.g., impulse, voltage, heart signal, etc.) can be applied to the two or more electrodes. In some implementations, the electrical signal provides an indication of a status of tissue (e.g., tissue capture status, leaflet capture status, tissue engagement status, etc.) within the tissue engagement portion, tissue capture portion, and/or capture region.

In some implementations, a bioimpedance signal can be measured (e.g., responsive to an electrical signal applied to the two or more electrodes, etc.), the bioimpedance signal providing an indication of a status of tissue within the tissue engagement portion, tissue capture portion, and/or capture region.

In some implementations, the status includes or indicates under insertion of tissue in the tissue engagement portion, tissue capture portion, and/or capture region.

In some implementations, the status includes or indicates full insertion of tissue in the tissue engagement portion, tissue capture portion, and/or capture region.

In some implementations, the status includes or indicates over insertion of tissue in the tissue engagement portion, tissue capture portion, and/or capture region.

In some implementations, the status includes or indicates angled insertion of tissue in the tissue engagement portion, tissue capture portion, and/or capture region.

In some implementations, the status includes or indicates insertion of non-targeted tissue in the tissue engagement portion, tissue capture portion, and/or capture region. In some implementations, the non-targeted tissue includes chordae tendineae, etc.

In some implementations, the status includes or indicates insertion of tissue in the tissue engagement portion, tissue capture portion, and/or capture region, while the tissue engagement portion, tissue capture portion, and/or capture region is in an open configuration including the first surface and the second surface being apart from each other.

In some implementations, the indication of the status is configured to generate or be used to generate a visual indicator for a user of the status (e.g., of the tissue engagement status, of the tissue capture status, of the leaflet capture status, etc.). In some implementations, the visual indicator is configured to indicate one or more of no tissue insertion, under tissue insertion, full tissue insertion, and over tissue insertion. In some implementations, the visual indicator is configured to indicate one or more of no tissue insertion, under tissue insertion, full tissue insertion, over tissue insertion, angled tissue insertion, and non-targeted tissue insertion.

In some implementations, a system and/or a device (e.g., a treatment system, a repair system, a valve repair system, a treatment device, etc., which can be the same as or similar to other systems and/or devices herein) includes a tissue engagement portion or tissue capture portion (e.g., an anchor, a clasp, a clip, a clamp, multiple arms, multiple gripping members, two paddles, a clasp arm and a paddle arm, a gripping member and a paddle, etc.).

In some implementations, the tissue engagement portion or tissue capture portion includes a first surface (e.g., a surface of a clip arm, clasp arm, paddle, coaptation element, other component, etc.) and a second surface (e.g., a surface of a clip arm, clasp arm, paddle, coaptation element, other component, etc.) configured such that the first surface and the second surface can close and/or be moved closer together to engage and/or capture tissue in the tissue engagement portion or tissue capture portion (e.g., to capture a leaflet of a native valve in the tissue capture portion).

In some implementations, the tissue engagement portion or tissue capture portion includes a first arm (e.g., a clip arm, a clasp arm, a paddle, etc.) and/or a second arm (e.g., a clip arm, a clasp arm, a paddle, etc.) configured such that the first arm and the second arm can close and/or be moved closer together to engage and/or capture tissue in the tissue engagement portion or tissue capture portion (e.g., to capture a leaflet of a native valve in the tissue capture portion).

In some implementations, the first arm includes the first surface and/or the second arm includes the second surface.

In some implementations, the system, apparatus, and/or device is useable for repairing and/or treating a native valve of a patient or simulation. In some implementations, the tissue is a leaflet of the native valve.

In some implementations, the system, apparatus, and/or device includes multiple tissue engagement portions, tissue capture portions, and/or anchors.

In some implementations, the system, apparatus, and/or device includes a second tissue engagement portion or second anchor including a first surface (e.g., a surface of a clip arm, clasp arm, paddle, coaptation element, etc.) and a second surface (e.g., a surface of a clip arm, clasp arm, paddle, coaptation element, etc.) configured such that the first surface and the second surface can close and/or be moved closer together to engage and/or capture tissue (e.g., a second leaflet of a native valve, another portion of a leaflet, etc.) in the second tissue engagement portion or anchor (e.g., the second tissue engagement portion or anchor can act as a tissue capture portion). In some implementations, the second tissue engagement portion or second anchor includes a first arm (e.g., a clip arm, a clasp arm, a paddle, etc.) and/or a second arm (e.g., a clip arm, a clasp arm, a paddle, etc.) configured such that the first arm and the second arm can close and/or be moved closer together to engage and/or capture tissue (e.g., a second leaflet of a native valve, another portion of a leaflet, etc.) in the second tissue engagement portion or anchor (e.g., in a clasp, clip, etc.). In some implementations, the first arm can comprise the first surface and/or the second arm can comprise the second surface. The second tissue engagement portion or second anchor can be configured the same as or similar to the first tissue engagement portion.

In some implementations, the system, apparatus, and/or device includes a third tissue engagement portion or third anchor including a first surface (e.g., a surface of a clip arm, clasp arm, paddle, coaptation element, etc.) and a second surface (e.g., a surface of a clip arm, clasp arm, paddle, coaptation element, etc.) configured such that the first surface and the second surface can close and/or be moved closer together to engage and/or capture tissue (e.g., a third leaflet of a native valve, another portion of a leaflet, etc.) in the third tissue engagement portion or anchor (e.g., the third tissue engagement portion can act as a tissue capture portion and capture tissue). In some implementations, the third tissue engagement portion or third anchor includes a first arm (e.g., a clip arm, a clasp arm, a paddle, etc.) and/or a second arm (e.g., a clip arm, a clasp arm, a paddle, etc.) configured such that the first arm and the second arm can close and/or be moved closer together to engage and/or capture tissue (e.g., a third leaflet of a native valve, another portion of a leaflet, etc.) in the third tissue engagement portion or anchor (e.g., in a clasp, clip, etc.). In some implementations, the first arm can comprise the first surface and/or the second arm can comprise the second surface. The third tissue engagement portion or third anchor can be configured the same as or similar to the first tissue engagement portion and/or the second tissue engagement portion or anchor.

In some implementations, at least one of the (i) first surface and/or first arm and (ii) the second surface and/or second arm (e.g., of a first tissue capture portion and/or a second tissue capture portion, etc.) is movable to form a capture region therebetween for capturing the tissue (e.g., capturing a leaflet of the native valve).

In some implementations, two or more electrodes are coupled to the tissue engagement portion or tissue capture portion (e.g., to an anchor, to a clasp, etc.), wherein the system, apparatus, and/or device is configured such that: an electrical signal can be applied to the two or more electrodes.

In some implementations, a bioimpedance signal can be measured, e.g., based on the applied electrical signal.

In some implementations, the two or more electrodes include: a first electrode strip coupled to the first surface and/or first arm of the tissue engagement portion or tissue capture portion (e.g., anchor, clasp, etc.) and a second electrode strip coupled to the first surface and/or first arm of the tissue engagement portion or tissue capture portion (e.g., anchor, clasp, etc.).

In some implementations, the two or more electrodes include: a first electrode strip coupled to the first surface and/or first arm of the tissue engagement portion or tissue capture portion (e.g., anchor, clasp, etc.) near a first edge of the first surface and/or first arm, and a second electrode strip coupled to the first surface and/or first arm of the tissue engagement portion or tissue capture portion (e.g., anchor, clasp, etc.) near a second edge of the first surface and/or first arm, the second edge opposite the first edge.

In some implementations, the first electrode strip and the second electrode strip are parallel to each other and run along a length of the first surface and/or first arm.

In some implementations, the first electrode strip and the second electrode strip are offset a prescribed distance from a free edge of the first surface and/or first arm of the tissue engagement portion or tissue capture portion (e.g., anchor, clasp, etc.).

In some implementations, the prescribed distance is between 1-15 mm. In some implementations, the prescribed distance is between 2-10 mm. In some implementations, the prescribed distance is between 5-8 mm. In some implementations, the prescribed distance is at least 6 mm.

In some implementations, a first bioimpedance signal can be measured, e.g., based on an electrical signal applied to the first electrode strip, and a second bioimpedance signal can be measured, e.g., based on an applied electrical signal to the second electrode strip.

In some implementations, the first bioimpedance signal and the second bioimpedance signal indicate a status of the tissue between the first surface and/or first arm and the second surface and/or second arm of the tissue engagement portion or tissue capture portion (e.g., anchor, clasp, etc.) and/or indicate a status or deployment status of the tissue engagement portion of tissue capture portion (e.g., a distance between the first surface and/or first arm and the second surface and/or second arm, an indication the tissue engagement portion or tissue capture portion is closed or open, etc.).

In some implementations, a difference between the capture status indicated by the first bioimpedance signal and indicated by the second bioimpedance signal indicates an angled insertion of the tissue between the first surface and/or first arm and the second surface and/or second arm of the tissue engagement portion or tissue capture portion (e.g., anchor, clasp, etc.).

In some implementations, an average of the first bioimpedance signal and the second bioimpedance signal is used to determine the capture status of the tissue.

In some implementations, the first bioimpedance signal and the second bioimpedance signal provide a continuous indication of tissue insertion between the first surface and/or first arm and the second surface and/or second arm.

In some implementations, the continuous indication of the capture status is divided into quantized signal regions indicating four categories of capture status that include no insertion of the tissue, under insertion of the tissue, full insertion of the tissue, and over insertion of the tissue. Other status signals are also possible.

In some implementations, the system, apparatus, and/or device includes a reference electrode configured to enable bipolar measurements of the bioimpedance signal.

In some implementations, the bioimpedance signal can be measured in at least three configurations including the first electrode strip versus the reference electrode, the second electrode strip versus the reference node, and the first electrode strip versus the second electrode strip.

In some implementations, the two or more electrodes include: a first electrode coupled to the first surface and/or first arm of the tissue engagement portion or tissue capture portion near a free edge of the first surface and/or first arm, the free edge opposite a hinged edge coupled to a hinged edge of the second surface and/or second arm; and a second electrode coupled to the second surface and/or second arm of the tissue engagement portion or tissue capture portion near a free edge of the second surface and/or second arm, the free edge opposite the hinged edge.

In some implementations, the first electrode and the second electrode are configured to contact one another with the tissue engagement portion or tissue capture portion is closed.

In some implementations, bioimpedance signals measured with the first electrode and with the second electrode are configured to be used to determine a thickness of the tissue inserted into the tissue engagement portion or tissue capture portion.

In some implementations, the bioimpedance signals measured with the first electrode and with the second electrode are configured to be used to determine variation in thickness of the tissue as the tissue is inserted into the tissue engagement portion or tissue capture portion.

In some implementations, a cross-sectional map of the thickness of the tissue is generated based on the determined thickness and variation in thickness of the tissue.

In some implementations, bioimpedance signals are measured with the first electrode and with the second electrode while the tissue engagement portion or tissue capture portion is partially closed to approximate the first electrode and the second electrode to the tissue inserted into the tissue engagement portion or tissue capture portion.

In some implementations, a system, apparatus, and/or device (e.g., a treatment system, a repair system, a valve repair system, a treatment device, a repair device etc. which can be the same as or similar to other systems, apparatuses, and/or devices herein) includes an anchor portion.

In some implementations, the system, apparatus, and/or device includes a coaptation portion coupled to the anchor portion.

In some implementations, the coaptation portion includes an optional coaptation element.

In some implementations, the anchor portion includes a clasp (or other anchor or tissue capture portion) configured to capture tissue (e.g., a leaflet of a native valve, a membrane, muscle, etc.) in the clasp (or other anchor or tissue capture portion).

In some implementations, one or more flexible electrodes protrude away from the coaptation portion and a reference electrode.

In some implementations, the system, apparatus, and/or device is configured such that: an electrical signal can be applied to the one or more flexible electrodes.

In some implementations, a bioimpedance signal can be measured (e.g., based on or responsive to the applied electrical signal, etc.) to determine relative blood flow adjacent to the system, apparatus, and/or device.

In some implementations, the one or more flexible electrodes are configured to measure blood flow through a native valve, e.g., when the system, apparatus, and/or device is implanted in the native valve.

In some implementations, the one or more flexible electrodes are configured to detect leakage through the native valve.

In some implementations, the one or more flexible electrodes are configured to deflect in response to blood flowing past the one or more flexible electrodes.

In some implementations, the bioimpedance signal changes in response to deflection of the one or more flexible electrodes.

In some implementations, the change in bioimpedance signal correlates to an amount of deflection which correlates to a blood flow rate through the valve.

In some implementations, the bioimpedance signal is configured to decrease in response to regurgitation blood volume through the native valve.

In some implementations, the reference electrode is coupled to an actuation element, the actuation element coupled to the coaptation portion and to the anchor portion.

In some implementations, a system and/or a device (e.g., a valve repair system, apparatus, and/or device, which can be the same as or similar to other systems/devices herein) includes an anchor portion.

In some implementations, the system, apparatus, and/or device includes a coaptation portion coupled to the anchor portion.

In some implementations, the coaptation portion can optionally include a coaptation element.

In some implementations, the anchor portion includes a clasp (or other anchor or tissue capture portion) configured to capture tissue (e.g., a leaflet of a native valve, a membrane, muscle, etc.) in the clasp (or other anchor or tissue capture portion).

In some implementations, one or more electrodes are coupled to the anchor portion.

In some implementations, the system, apparatus, and/or device is configured such that: an electrical signal can be applied to the one or more electrodes.

In some implementations, a bioimpedance signal can be measured (e.g., based on the applied electrical signal, etc.) to determine forces on the system, apparatus, and/or device.

In some implementations, the bioimpedance signal is correlated with a deflection of the coaptation portion or the anchor portion.

In some implementations, the deflection is correlated with a force applied to the system, apparatus, and/or device such that the bioimpedance signal is correlated with the force applied to the system, apparatus, and/or device.

In some implementations, the anchor portion include an inner paddle coupled to an outer paddle that rotate relative to one another, wherein forces applied to the system, apparatus, and/or device change an opening distance between the inner paddle and the outer paddle.

In some implementations, a first electrode of the two or more electrodes is coupled to the inner paddle and a second electrode of the two or more electrodes is coupled to the outer paddle such that the change in the opening distance causes a change in the bioimpedance signal.

In some implementations, a system, an apparatus, and/or a device (e.g., a treatment system, a repair system, a valve repair system, a treatment device, a repair device, etc., which can be the same as or similar to other systems and/or devices herein) includes at least one tissue engagement portion or anchor (e.g., a helical anchor, a screw, a staple, a dart, a hook, a clasp, a clip, a clamp, multiple arms, multiple gripping members, two paddles, a clasp arm and a paddle arm, a gripping member and a paddle, etc.).

In some implementations, the tissue engagement portion or anchor includes a first surface (e.g., a surface of a clip arm, clasp arm, paddle, etc.) and a second surface (e.g., a surface of a clip arm, clasp arm, paddle, etc.) configured such that the first surface and the second surface can close or be moved closer together to engage and/or capture tissue (e.g., a leaflet of a native valve, a membrane, muscle, etc.) in the tissue engagement portion or anchor (e.g., the tissue engagement portion or anchor can act as a tissue capture portion and capture the tissue).

In some implementations, the tissue engagement portion or anchor includes a first arm (e.g., a clip arm, a clasp arm, a paddle, etc.) and/or a second arm (e.g., a clip arm, a clasp arm, a paddle, etc.) configured such that the first arm and the second arm can close or be moved closer together to engage and/or capture tissue (e.g., a leaflet of a native valve, a membrane, muscle, etc.) in the tissue engagement portion or anchor (e.g., the tissue engagement portion or anchor can act as a tissue capture portion and capture the tissue). In some implementations, the first arm comprises the first surface and/or the second arm comprises the second surface.

In some implementations, at least one of the first surface and/or first arm and the second surface and/or second arm are movable to form a capture region therebetween for capturing the tissue (e.g., leaflet of the native valve).

In some implementations, a plurality of electrodes is coupled to the tissue engagement portion or anchor.

In some implementations, the plurality of electrodes electrically are coupled in series using an electrical lead.

In some implementations, a plurality of electrical components are electrically coupled in series with the plurality of electrodes using the electrical lead.

In some implementations, the system, apparatus, and/or device is configured such that: an electrical signal can be applied to the plurality of electrodes through the electrical lead.

In some implementations, a bioimpedance signal can be measured for the plurality of electrodes (e.g., based on the applied electrical signal, etc.).

In some implementations, measured bioimpedance values can be determined for each electrode of the plurality of electrodes based on electrical characteristics of the plurality of electrical components and the electrical signal.

In some implementations, one or more electrical components of the plurality of electrical components is coupled in series between a pair of electrodes of the plurality of electrodes using the electrical lead.

In some implementations, the one or more electrical components includes a resistor, capacitor, or inductor.

In some implementations, the one or more electrical components in series between the pair of electrodes of the plurality of electrodes have different electrical characteristics than another of the one or more electrical components in series between a different pair of electrodes of the plurality of electrodes.

In some implementations, the electrical signal can be applied with a predetermined current and frequency.

In some implementations, the electrical lead includes a single electrical lead.

In some implementations, a resistor with a fixed resistance value is coupled in series between a first electrode and a second electrode of the plurality of electrodes.

In some implementations, a capacitor with a fixed capacitance value is coupled in series between the second electrode and a third electrode of the plurality of electrodes.

In some implementations, an inductor with a fixed inductance value is coupled in series between the third electrode and a fourth electrode of the plurality of electrodes.

In some implementations, the measured bioimpedance signals of the first, second, third, and fourth electrodes of the plurality of electrodes depends on the fixed resistance of the resistor, the fixed capacitance of the capacitor, and the fixed inductance of the inductor in conjunction with a predetermined frequency and current of the applied electrical signal.

In some implementations, a system, an apparatus, and/or a device (e.g., a treatment system, a repair system, a valve repair system, a treatment device, a repair device, which can be the same as or similar to other systems, apparatuses, and/or device herein) includes a tissue engagement portion or anchor (e.g., a helical anchor, screw, staple, dart, hook, clasp, clip, clamp, multiple arms, multiple gripping members, two paddles, a clasp arm and a paddle arm, a gripping member and a paddle, etc.).

In some implementations, the tissue engagement portion or anchor includes a first arm (e.g., a clip arm, a clasp arm, a paddle, etc.) and/or a second arm (e.g., a clip arm, a clasp arm, a paddle, etc.) configured such that the first arm and the second arm can close or be moved closer together to engage and/or capture tissue (e.g., a leaflet of a native valve, a membrane, muscle, etc.) in the tissue engagement portion or anchor (e.g., the tissue engagement portion or anchor can act as a tissue capture portion and capture the tissue). In some implementations, at least one of the first arm and the second arm is movable to form a capture region therebetween for capturing the tissue (e.g., leaflet, etc.) and/or movable closer to the other arm to capture tissue therebetween.

In some implementations, a plurality of electrodes is coupled to the tissue engagement portion or anchor.

In some implementations, an analog-to-digital converter (ADC) chip is coupled to the tissue engagement portion or anchor and electrically coupled to the plurality of electrodes.

In some implementations, an electrical lead (e.g., a single electrical lead, etc.) is configured to direct signals from the ADC chip to a measurement system.

In some implementations, the system, apparatus, and/or device is configured such that: an electrical signal can be applied to the plurality of electrodes through the ADC chip, the ADC chip digitizes a bioimpedance signal from each of the plurality of electrodes, the digitized bioimpedance signal is transmitted over the single electrical lead to the measurement system, and the bioimpedance signal is determined for each of the plurality of electrodes based on the applied electrical signal and the digitized bioimpedance signal.

In some implementations, the digitized bioimpedance signal is sent over the single electrical lead using digital packets.

In some implementations, the system, apparatus, and/or device (which can be the same as or similar to other systems and/or devices herein) includes at least one tissue engagement portion or anchor (e.g., a clasp, a clip, a clamp, multiple arms, multiple gripping members, two paddles, a clasp arm and a paddle arm, a gripping member and a paddle, etc.).

In some implementations, the tissue engagement portion or anchor includes a first surface (e.g., a surface of a clip arm, clasp arm, paddle, coaptation element, etc.) and a second surface (e.g., a surface of a clip arm, clasp arm, paddle, coaptation element, etc.) configured such that the first surface and the second surface can close or be moved closer together to engage and/or capture tissue (e.g., a leaflet of a native valve, a membrane, muscle, etc.) in the tissue engagement portion or anchor (e.g., the tissue engagement portion or anchor can act as a tissue capture portion and capture the tissue). In some implementations, at least one of the first surface and the second surface is movable to form a capture region therebetween for capturing the tissue (e.g., leaflet, etc.) and/or moveable closer to the other arm to capture tissue therebetween.

In some implementations, the tissue engagement portion or anchor includes a first arm (e.g., a clip arm, a clasp arm, a paddle, etc.) and/or a second arm (e.g., a clip arm, a clasp arm, a paddle, etc.) configured such that the first arm and the second arm can close or be moved closer together to engage and/or capture tissue (e.g., a leaflet of a native valve, a membrane, muscle, etc.) in the tissue engagement portion or anchor (e.g., the tissue engagement portion or anchor can act as a tissue capture portion and capture the tissue). In some implementations, at least one of the first arm and the second arm is movable to form a capture region therebetween for capturing the tissue (e.g., leaflet, etc.) and/or movable closer to the other arm to capture tissue therebetween. In some implementations, the first arm includes the first surface and/or the second arm includes the second surface.

In some implementations, a flexible printed circuit board (PCB) includes a body, one or more electrodes coupled to the body, and an electrical lead extending away from the body, and the flexible PCB is coupled to the tissue engagement portion or anchor (e.g., to a clasp, clip, paddle, etc.).

In some implementations, the flexible PCB is coupled to the tissue engagement portion or anchor using one or more sutures. Other coupling mechanisms are also possible.

In some implementations, the flexible PCB is coupled to the tissue engagement portion or anchor the system, apparatus, and/or device is configured such that: an electrical signal can be applied to the one or more electrodes through the electrical lead of the flexible PCB, and a bioimpedance signal can be measured using the electrical lead based on or in response to the applied electrical signal.

In some implementations, the system, apparatus, and/or device includes a cover that covers the tissue engagement portion or anchor, wherein the flexible PCB is secured to the cover that covers the anchor to couple the flexible PCB to the tissue engagement portion or anchor.

In some implementations, the system, apparatus, and/or device includes a cover that covers the tissue engagement portion or anchor, wherein the flexible PCB is secured to the first arm of the tissue engagement portion or anchor under the cover that covers the tissue engagement portion or anchor to couple the flexible PCB to the tissue engagement portion or anchor.

In some implementations, the flexible PCB includes one or more physical features that facilitate removal of the flexible PCB from the tissue engagement portion or anchor by applying a force to the electrical lead.

In some implementations, the one or more physical features include a stress concentration point including a narrow connection point between two openings, the one or more sutures is configured to extend through the two openings and over the narrow connection point to couple the flexible PCB to the tissue engagement portion or anchor, and applying a force to the electrical lead causes the narrow connection point to break thereby releasing the flexible PCB from the tissue engagement portion or anchor and leaving the one or more sutures coupled to the tissue engagement portion or anchor.

In some implementations, the one or more physical features include a Y-shaped protrusion extending from an end of the body the flexible PCB opposite an end from which the electrical lead extends away from the body of the flexible PCB, the Y-shaped protrusion includes a pair of legs extending away from a bridge portion that extends away from the body of the flexible PCB.

In some implementations, the bridge portion forms a rotation cutout configured to facilitate rotation of the pair of legs toward one another with an inward force applied to the pair of legs. In some implementations, the one or more sutures are configured to extend over the bridge portion to secure the flexible PCB to the tissue engagement portion or anchor.

In some implementations, applying a force to the electrical lead causes the suture to push the pair of legs toward one another to allow the flexible PCB to slide from under the suture thereby releasing the flexible PCB from the tissue engagement portion or anchor and leaving the one or more sutures coupled to the tissue engagement portion or anchor.

In some implementations, the one or more physical features include a round protrusion extending from a side of the body the flexible PCB. In some implementations, the round protrusion includes a neck portion that connects the round portion to the body of the flexible PCB.

In some implementations, the round protrusion is configured to wrap over a side of the tissue engagement portion or anchor and the one or more sutures are configured to extend over the neck portion at the side of the tissue engagement portion or anchor to secure the flexible PCB to the tissue engagement portion or anchor.

In some implementations, applying a force to the electrical lead causes the round protrusion to deform to allow the flexible PCB to slide from under the suture thereby releasing the flexible PCB from the tissue engagement portion or anchor and leaving the one or more sutures coupled to the tissue engagement portion or anchor.

In some implementations, the one or more physical features include a pair of side indents formed from the body of the flexible PCB, the side indents formed in opposite sides of the body of the flexible PCB.

In some implementations, the side indents are configured to provide a positive lock in a targeted location of the flexible PCB, the targeted location configured so as to not interfere with measurements made with the one or more electrodes of the flexible PCB.

In some implementations, applying a force to the electrical lead causes the flexible PCB to slide from under the suture thereby releasing the flexible PCB from the tissue engagement portion or anchor and leaving the one or more sutures coupled to the tissue engagement portion or anchor.

In some implementations, the one or more physical features include a hole formed in the body of the flexible PCB near an edge of the body of the flexible PCB opposite an end from which the electrical lead extends away from the body of the flexible PCB. In some implementations, the one or more sutures are configured to pass through the hole over the body of the flexible PCB to secure the flexible PCB to the tissue engagement portion or anchor.

In some implementations, applying a force to the electrical lead causes the body of the PCB to break at the edge of the body of the flexible PCB thereby releasing the flexible PCB from the tissue engagement portion or anchor and leaving the one or more sutures coupled to the tissue engagement portion or anchor.

In some implementations, the one or more physical features includes a relief extending from the hole to the edge. In some implementations, the relief is configured to allow the one or more sutures to pass through the relief to release the flexible PCB from the tissue engagement portion or anchor.

In some implementations, the one or more physical features include a pair of bi-directional tongues that form a pair of tabs on the body of the flexible PCB, the pair of tabs being oriented in opposite directions from one another, each of the pair of tabs being is configured to allow a suture of the one or more sutures to pass over a portion of the body of the flexible PCB and under the tabs to secure the flexible PCB to the tissue engagement portion or anchor.

In some implementations, applying a force to the electrical lead causes the one or more sutures to push the corresponding tab away from the body of the PCB to allow the flexible PCB to slide from under the one or more sutures thereby releasing the flexible PCB from the tissue engagement portion or anchor and leaving the one or more sutures coupled to the tissue engagement portion or anchor.

In some implementations, a system, an apparatus, and/or a device (e.g., a treatment system, a repair system, a valve repair system, a treatment device, a repair device etc., which can be the same as or similar to other systems and/or devices herein) includes a tissue engagement portion or anchor (e.g., a helical anchor, screw, dart, staple, hook, clasp, clip, clamp, multiple arms, multiple gripping members, two paddles, a clasp arm and a paddle arm, a gripping member and a paddle, etc.).

In some implementations, the tissue engagement portion or anchor includes a first surface (e.g., a surface of a clip arm, clasp arm, paddle, etc.) and a second surface (e.g., a surface of a clip arm, clasp arm, paddle, etc.) configured such that the first surface and the second surface can close or be moved closer together to engage and/or capture tissue (e.g., a leaflet of a native valve, membrane, lining, muscle, etc.) in the tissue engagement portion or anchor (e.g., the tissue engagement portion or anchor can act as a tissue capture portion and capture the tissue).

In some implementations, the tissue engagement portion or anchor includes a first arm (e.g., a clip arm, a clasp arm, a paddle, etc.) and/or a second arm (e.g., a clip arm, a clasp arm, a paddle, etc.) configured such that the first arm and the second arm can close or be moved closer together to engage and/or capture tissue (e.g., a leaflet of a native valve, membrane, lining, muscle, etc.) in the tissue engagement portion or anchor (e.g., the tissue engagement portion or anchor can act as a tissue capture portion and capture the tissue). In some implementations, the first arm comprises the first surface and/or the second arm comprises the second surface.

In some implementations, at least one of the first arm and the second arm is movable to form a capture region therebetween for capturing the tissue (e.g., leaflet, etc.).

In some implementations, the tissue engagement portion or anchor includes a plurality of barbs to secure the tissue (e.g., leaflet, etc.) within the tissue engagement portion or anchor.

In some implementations, the tissue engagement portion or anchor includes a flexible printed circuit board (PCB) including an electrode pad or electrode array with one or more electrodes coupled to the electrode pad. In some implementations, an electrical lead extends away from the electrode pad/array.

In some implementations, the system, apparatus, and/or device is configured such that: an electrical signal can be applied to the one or more electrodes through the electrical lead of the flexible PCB. In some implementations, a bioimpedance signal can be measured using the electrical lead based on or in response to the applied electrical signal.

In some implementations, application of a force to the electrical lead causes the flexible PCB and/or the one or more electrodes to be removed from the system, apparatus, and/or device.

In some implementations, the flexible PCB is configured to be pulled through a pair of barbs of the plurality barbs to remove the flexible PCB from the system, apparatus, and/or device.

In some implementations, the electrical lead extends between the pair of barbs.

In some implementations, the electrode pad of the flexible PCB has a width that is greater than a distance between the pair of barbs, the electrode pad of the flexible PCB configured to bend to fit between the pair of barbs.

In some implementations, the width of the electrode pad is less than or equal to 1.875 times the distance between the pair of barbs.

In some implementations, the width of the electrode pad is less than or equal to 1.25 times the distance between the pair of barbs.

In some implementations, the distance between the pair of barbs is less than or equal to 8 mm.

In some implementations, a force required to pull the electrode pad through the pair of barbs is less than or equal to 1.5 N.

In some implementations, the flexible PCB is configured to be pulled around a side of the plurality barbs to remove the flexible PCB from the system, apparatus, and/or device.

In some implementations, the electrical lead has a diagonal bend section leading immediately away from the electrode pad such that the electrode pad is laterally offset from the electrical lead so that the electrical lead lies along the side of the plurality of barbs while the electrode pad is within the tissue engagement portion or anchor.

In some implementations, pulling on the electrical lead causes the electrode pad to exit the tissue engagement portion or anchor from a side of the tissue engagement portion or anchor around the plurality of barbs.

In some implementations, pulling on the electrical lead causes the diagonal bend section to contact the plurality of barbs so as to cause the electrode pad to move laterally relative to the plurality of barbs to exit the side of the tissue engagement portion or anchor, using one or more barbs of the plurality of barbs as a fulcrum.

In some implementations, the electrode pad includes a relief cut through the electrode pad such that application of a sufficient force causes the electrode pad to split apart into a first lateral portion and a second lateral portion.

In some implementations, the flexible PCB includes a second electrical lead, the electrical lead coupled to the first lateral portion of the electrode pad and the second electrical lead coupled to the second lateral portion of the electrode pad.

In some implementations, the electrical lead and the second electrical lead each include diagonal bend sections in opposite directions so that the electrical lead and the second electrical lead are each laterally offset from the respective lateral portion of the electrode pad so that the electrical lead lies along a first side of the plurality of barbs and the second electrical lead lies along a second side of the plurality of barbs opposite the first side while the electrode pad is within the tissue engagement portion or anchor.

In some implementations, application of a proximal force on the electrical lead and the second electrical lead causes the electrode pad to split into the first lateral portion and the second lateral portion.

In some implementations, application of the proximal force on the electrical lead and the second electrical lead after the electrode split in the first lateral portion and the second lateral portion causes the first lateral portion to exit the tissue engagement portion or anchor around the first side of the plurality of barbs and causes the second lateral portion to exit the tissue engagement portion or anchor around the second side of the plurality of barbs.

In some implementations, the flexible PCB includes a reference electrode coupled to the electrical lead.

In some implementations, the electrical lead is configured to extend proximally to a proximal end of a delivery system configured to implant the system, apparatus, and/or device.

In some implementations, a system, an apparatus, and/or a device (e.g., a treatment system, a repair system, a valve repair system, a treatment device, a repair device, which can be the same as or similar to other systems, apparatuses, and/or devices herein) includes an anchor portion including a tissue engagement portion or anchor (e.g., a clasp, a clip, a clamp, multiple arms, multiple gripping members, two paddles, a clasp arm and a paddle arm, a gripping member and a paddle, etc.).

In some implementations, the tissue engagement portion or anchor includes a first surface (e.g., a surface of a clip arm, clasp arm, paddle, etc.) and a second surface (e.g., a surface of a clip arm, clasp arm, paddle, etc.) configured to engage (e.g., capture, attach to, etc.) tissue (e.g., a leaflet of a native valve, membrane, lining, muscle, etc.).

In some implementations, the tissue engagement portion or anchor includes a first arm (e.g., a clip arm, a clasp arm, a paddle, etc.) and/or a second arm (e.g., a clip arm, a clasp arm, a paddle, etc.) configured to engage (e.g., capture, attach to, etc.) tissue (e.g., a leaflet of a native valve, membrane, lining, muscle, etc.). In some implementations, the first arm comprises the first surface and/or the second arm comprises the second surface.

In some implementations, the system, apparatus, and/or device includes a distal portion configured to engage with an actuation element (e.g., wire, line, suture, tube, rod, etc.) of a delivery system. In some implementations, the actuation element is configured to rotate to deploy the anchor portion.

In some implementations, the system, apparatus, and/or device includes an electrode coupled to the tissue engagement portion or anchor.

In some implementations, one or more wires are coupled to the electrode and coupled to the actuation element of the delivery system.

In some implementations, the system, apparatus, and/or device is configured such that: an electrical signal can be applied to the electrode through the one or more wires, and a bioimpedance signal can be measured based on or in response to the applied electrical signal.

In some implementations, rotation of the actuation element of the delivery system causes the one or more wires to spool around the actuation element so as to pull the electrode off of the tissue engagement portion or anchor to remove the electrode from the system, apparatus, and/or device.

In some implementations, the one or more wires are secured to a collar that is affixed to the actuation element such that rotation of the actuation element causes the collar to rotate.

In some implementations, one or more electrical leads are coupled to the one or more wires at the collar to provide electrical connectivity to a proximal end of the delivery system.

In some implementations, the electrode includes a flexible printed circuit board.

In some implementations, the electrode is releasably secured to the tissue engagement portion or anchor.

In some implementations, rotation of the actuation element further causes the electrode to spool around the actuation element, thereby removing the electrode and the one or more wires from the system, apparatus, and/or device.

In some implementations, methods and/or techniques described herein relate to operation or use of a system (which can be a system useable for repairing and/or treating a native valve of a patient or simulation and can be the same as or similar to other systems herein), the system including a delivery system and a valve repair device.

In some implementations, the methods and/or techniques comprise using the system to access an interior of a body and repair and/or treat tissue of the body. In some implementations, the methods and/or techniques comprise using the system to repair and/or treat a heart valve of the body.

In some implementations, the methods and/or techniques comprise using the delivery system to advance the valve repair device to a heart valve and deploy or otherwise use the valve repair device to repair and/or treat the heart valve. In some implementations, deploying or otherwise using the valve repair device to repair and/or treat the heart valve includes anchoring the valve repair device to tissue of the heart and/or heart valve.

In some implementations, the delivery system includes: a catheter with a proximal end and a distal end, an actuation element, a wire extending within a lumen of the catheter from the proximal end of the catheter to the distal end of the catheter, and/or a capture mechanism at a distal end of the delivery system.

In some implementations, the valve repair device includes: an attachment portion including a proximal component (e.g., a proximal collar, proximal ring, proximal extension, etc.) configured to engage with the capture mechanism of the delivery system and an anchor portion including a tissue engagement portion or anchor (e.g., helical anchor, screw, dart, staple, hook, clasp, clip, clamp, multiple arms, multiple gripping members, two paddles, a clasp arm and a paddle arm, a gripping member and a paddle, a combination of these, etc.).

In some implementations, the tissue engagement portion or anchor includes a first surface (e.g., a surface of an anchor, anchor head, clip arm, clasp arm, paddle, etc.) configured to engage tissue (e.g., an annulus of a native valve, a leaflet of a native valve, a membrane, a lining, muscle, etc.).

In some implementations, the tissue engagement portion or anchor includes a first surface (e.g., a surface of a clip arm, clasp arm, paddle, etc.) and a second surface (e.g., a surface of a clip arm, clasp arm, paddle, etc.) configured to capture tissue (e.g., a leaflet of a native valve, membrane, lining, muscle, etc.).

In some implementations, the tissue engagement portion or anchor includes a first arm (e.g., a clip arm, a clasp arm, a paddle, etc.) and/or a second arm (e.g., a clip arm, a clasp arm, a paddle, etc.) configured to capture tissue (e.g., a leaflet of a native valve, membrane, lining, muscle, etc.). In some implementations, the first arm comprises the first surface and/or the second arm comprises the second surface.

In some implementations, the valve repair device includes a distal portion configured to engage with the actuation element of the delivery system, the actuation element configured to deploy the anchor portion.

In some implementations, the actuation element is also configured to release the capture mechanism from the proximal component.

In some implementations, an electrode is coupled to the tissue engagement portion or anchor (e.g., coupled to a first surface thereof, etc.)

In some implementations, an electrical lead has a distal end coupled to the electrode and a proximal end coupled to the proximal component.

In some implementations, the valve repair device is configured such that: an electrical signal can be applied to the electrode through the electrical lead, and a bioimpedance signal can be measured based on or in response to the applied electrical signal.

In some implementations, the wire is configured to provide an electrical connection to the electrical lead during delivery and deployment of the valve repair device that is terminated upon withdrawal of the delivery system.

In some implementations, a distal end of the wire includes a spring pin connector, the proximal end of the electrical lead is coupled to an electrical pad at the proximal component, and the spring pin connector of the wire is in electrical contact with the electrical pad of the electrical lead to provide electrical connection to the electrode until the valve repair device is released from the delivery system.

In some implementations, a distal end of the wire includes an electrical pad, the proximal end of the electrical lead is coupled to a spring pin connector at the proximal component, and the spring pin connector of the electrical lead is in electrical contact with the electrical pad of the wire to provide electrical connection to the electrode until the valve repair device is released from the delivery system.

In some implementations, the spring pin connector is configured to use spring forces parallel to a shaft of the catheter to provide electrical contact between the electrical lead and the wire.

In some implementations, a spring force of the spring pin connector is configured to assist in detaching the spring pin connector from the electrical pad.

In some implementations, the proximal component forms a groove, the electrical lead is coupled to the proximal component within the groove.

In some implementations, the capture mechanism includes a finger configured to mate with the groove of the proximal component to couple the valve repair device to the delivery system. In some implementations, the wire is coupled to an inner surface of the finger so that the wire physically contacts the electrical lead in the groove to provide electrical contact between the wire and the electrical lead.

In some implementations, release of the valve repair device from the delivery system causes the finger to disengage from the proximal component, thereby releasing the valve repair device and terminating electrical contact between the wire and the electrical lead.

In some implementations, the groove and the finger are coated with an insulative material to electrically isolate the electrical connection between the wire and the electrical lead.

In some implementations, the delivery system includes a tube coupled to the capture mechanism with the wire secured within the tube, the proximal end of the electrical lead is releasably secured within the tube to provide electrical contact between the wire and the electrical lead while the valve repair device is coupled to the delivery system.

In some implementations, withdrawal of the delivery system from the valve repair device causes the tube to move away from the proximal component, thereby releasing the electrical lead from the tube and terminating electrical contact between the wire and the electrical lead.

In some implementations, the tube includes a leaf spring to provide a clamping force on the wire and the electrical lead to enhance the electrical connection.

In some implementations, the delivery system includes a frame secured to the distal end of the catheter, the tube being coupled to the frame and the frame configured to hold the tube in a targeted location relative to the valve repair device.

In some implementations, the frame is made of a polymer to electrically isolate the electrical connection between the wire and the electrical lead.

In some implementations, the frame includes a U-shaped support that engages with the attachment portion of the valve repair device.

In some implementations, a distal end of the wire terminates with a coil crimp having an inner diameter, the proximal end of the electrical lead is seated within the coil crimp, the inner diameter configured to provide a friction fit between the electrical lead and the wire to establish an electrical connection between the wire and the electrical lead, and the coil crimp is configured to expand to release the electrical lead.

In some implementations, the coil crimp is configured to expand responsive to being exposed to a temperature above a threshold temperature.

In some implementations, the coil crimp is configured to expand responsive to a current above a threshold current being driven through the wire.

In some implementations, the coil crimp is formed with a shape memory alloy in a martensite state, the inner diameter being smaller than a diameter of the electrical lead.

In some implementations, the coil crimp is configured to expand to have an inner diameter larger than the diameter of the electrical lead responsive to transitioning to the austenite state.

In some implementations, the coil crimp includes a bent location to enhance a friction fit between the wire and the electrical lead.

In some implementations, the electrical lead is inserted into the coil crimp at the bent location.

In some implementations, the capture mechanism includes a pair of fingers that are configured to engage with the proximal component to releasably secure the valve repair device to the delivery system.

In some implementations, the capture mechanism includes a disc crimp having a first section coupled to a first finger of the pair of fingers and a second section coupled to a second finger of the pair of fingers. In some implementations, the first section and the second section of the disc crimp forming a connection channel when abutted by the pair of fingers.

In some implementations, the connection channel opening when the first section and the second section are separated, the wire is coupled to the connection channel and the electrical lead is seated within the connection channel, the connection channel is sized to force the wire to physically contact the electrical lead to form an electrical connection.

In some implementations, releasing the valve repair device from the delivery system causes the pair of fingers to separate from the proximal component and to separate the first section from the second section of the disc crimp, thereby allowing the wire and the electrical lead to separate to terminate the electrical connection.

In some implementations, the disc crimp includes a polymer that is configured to electrically insulate the electrical connection between the wire and the electrical lead.

In some implementations, the first section is coupled to the first finger by inserting a portion of the first section through a window of the first finger to establish a friction fit between the first section and the first finger, and the second section is coupled to the second finger by inserting a portion of the second section through a window of the second finger to establish a friction fit between the second section and the second finger.

In some implementations, the first section and the second section include a shape set alloy that is welded to the first finger and the second finger, respectively.

In some implementations, the connection channel is coated with an electrically insulative coating to electrically insulate the electrical connection between the wire and the electrical lead.

In some implementations, the delivery system includes a heat-activated electrical connector coupled to the capture mechanism with the wire secured within the heat-activated electrical connector, the proximal end of the electrical lead is releasably secured within the heat-activated electrical connector to provide electrical contact between the wire and the electrical lead while the valve repair device is coupled to the delivery system.

In some implementations, the heat-activated electrical connector is configured to change shape responsive to the application of heat or current, the change in shape configured to release the electrical lead from the heat-activated electrical connector.

In some implementations, withdrawal of the delivery system from the valve repair device includes applying heat or current to the heat-activated electrical connector to cause the heat-activated electrical connector to open to release the electrical lead, thereby releasing the electrical lead from the heat-activated electrical connector and terminating electrical contact between the wire and the electrical lead.

In some implementations, the heat-activated electrical connector includes a shape set alloy with a transition temperature above average body temperature.

In some implementations, the heat-activated electrical connector is heated using heated saline.

In some implementations, the heat-activated electrical connector is opened by applying a current via the wire.

In some implementations, the heat-activated electrical connector includes a flat tube with an open orifice that is configured to transition to an open U-shape responsive to the application of heat or current above a threshold to enable removal of the electrical lead.

In some implementations, the heat-activated electrical connector includes a flat tube that is configured to transition to an open cylinder responsive to the application of heat or current above a threshold to enable removal of the electrical lead.

In some implementations, a distal end of the wire includes a shape memory alloy that is formed into a shepherd hook and configured to transition to a straight wire with application of heat or current. In some implementations, a proximal end of the electrical lead includes a shape memory alloy that is formed into a shepherd hook and configured to transition to a straight wire with application of heat or current. In some implementations, the shepherd hook of the wire and the shepherd hook of the electrical lead are hooked to each other to form an electrical connection.

In some implementations, withdrawal of the delivery system from the valve repair device includes applying heat or current to the distal end of the wire and to the proximal end of the electrical lead to cause the wire and the electrical lead straighten, thereby disconnecting the electrical lead and the wire to terminate the electrical connection between the wire and the electrical lead.

In some implementations, the heat-activated electrical connector is heated using heated saline.

In some implementations, the heat-activated electrical connector is opened by applying a current via the wire.

In some implementations, the wire includes a first portion including a first metal and a second portion including the shape memory alloy, the first portion joined to the second portion using a first crimp, and the electrical lead includes a first portion including the first metal and a second portion including the shape memory alloy, the first portion joined to the second portion using a second crimp.

In some implementations, the methods and/or techniques described herein relate to a device that includes a tissue engagement portion or tissue capture portion including a first surface and a second surface. In some implementations, the methods and/or techniques described herein relate to using the device at a tissue site in a body. In some implementations, the methods and/or techniques described herein relate to using the device at a heart valve in a heart. In some implementations, the methods and/or techniques described herein relate to advancing the device inside a heart and deploying (e.g., anchoring, etc.) the device at a heart valve in a heart.

In some implementations, the tissue capture portion is configured such that the first surface and the second surface can close or be moved closer together to capture tissue in the tissue capture portion.

In some implementations, at least one of the first surface and the second surface is movable to form a capture region between the first surface and the second surface for capturing the tissue and/or movable closer together to capture tissue therebetween.

In some implementations, two or more electrodes are coupled to the tissue capture portion. In some implementations, the device is configured such that: an electrical signal can be applied to the two or more electrodes, and a bioimpedance signal can be measured responsive to the electrical signal applied. In some implementations, the bioimpedance signal provides an indication of a status of the tissue within the tissue capture portion and/or an indication of a status of the tissue capture portion (e.g., a distance between a first surface and/or first arm and a second surface and/or second arm, an indication the tissue capture portion is closed or open, etc.).

In some implementations, the two or more electrodes include a first electrode coupled to the first surface and a second electrode coupled to the second surface.

In some implementations, the first electrode is adjacent to the second electrode when the tissue capture portion is in a closed configuration.

In some implementations, the first electrode includes an electrode plate covering a majority of the first surface and the second electrode includes an electrode plate covering a majority of the second surface.

In some implementations, the two or more electrodes include a first electrode coupled to the first surface and a second electrode coupled to the first surface.

In some implementations, the first electrode is separated from the second electrode by a gap.

In some implementations, the first electrode and the second electrode include electrode strips parallel to a length of the first surface.

In some implementations, the first electrode and the second electrode include electrode strips parallel to a width of the first surface.

In some implementations, the first electrode is positioned on the first surface at a first tissue capture depth.

In some implementations, the second electrode is positioned on the first surface at a second tissue capture depth greater than the first tissue capture depth.

In some implementations, the device includes an electrode plate coupled to the second surface.

In some implementations, the device includes an impedance measurement device configured to measure bioimpedance signals and to determine a tissue capture depth based on the measured bioimpedance signals.

In some implementations, the impedance measurement device implements an algorithm to generate an indicator of fully captured tissue, partially captured tissue, or overly captured tissue.

In some implementations, the impedance measurement device implements an algorithm to generate an indicator of tissue capture depth.

In some implementations, the impedance measurement device is configured to generate an indicator of the status of the tissue when the tissue capture portion is in a closed configuration and/or of the status of the tissue capture portion (e.g., a distance between a first surface and/or first arm and a second surface and/or second arm, an indication the tissue engagement portion or tissue capture portion is closed or open, etc.).

In some implementations, the impedance measurement device is configured to generate an indicator of the status of the tissue when the tissue capture portion is in an open configuration.

In some implementations, the tissue capture portion is configured as or to include one or more of the following: an anchor, a clasp, a clip, a clamp, a gripper, a gripping member, paddles, arms, combinations of these, etc.

In some implementations, the methods and/or techniques described herein relate to a device (e.g., an implantable device configured to be implanted during a medical procedure, a treatment device configured to be used in a medical procedure even if not necessarily implanted, etc.) that includes a tissue engagement portion or anchor configured to secure the device to tissue in a patient. In some implementations, at least one electrode is coupled to the tissue engagement portion or anchor.

In some implementations, the device is configured such that: an electrical signal can be applied to the tissue engagement portion or anchor, and a bioimpedance signal can be measured based on or in response to the applied electrical signal. In some implementations, the bioimpedance signal provides an indication of a status or deployment status of the tissue engagement portion or anchor and/or a status of the tissue relative to the tissue engagement portion or anchor.

In some implementations, the device comprises an annuloplasty device.

In some implementations, the device further comprises a plurality of anchors, each anchor including at least one electrode.

In some implementations, an electrical signal can be applied to the plurality of anchors and a bioimpedance signal can be measured from each of the plurality of anchors based on or in response to the applied electrical signal, each bioimpedance signal configured to indicate a deployment status of the corresponding anchor of the plurality of anchors.

In some implementations, the device includes a plurality of anchors that are electrically shorted together.

In some implementations, the device includes an impedance measurement device configured to measure bioimpedance signals and to determine an anchor deployment status based on measured bioimpedance signals.

In some implementations, the impedance measurement device implements an algorithm to generate an indicator of anchor deployment status, the anchor deployment status including the anchor in contact with tissue, a partially deployed anchor, and a fully deployed anchor.

In some implementations, the methods and/or techniques described herein relate to a system, an apparatus, and/or a device (e.g., a treatment system, a repair system, a valve repair system, a treatment device, a repair device, etc.) usable for repairing and/or treating a native valve and/or other tissue of a patient or simulation. In some implementations, the methods and/or techniques described include using the system, apparatus, and/or device to repair and/or treat a native valve and/or other tissue of a patient or simulation.

In some implementations, the system, apparatus, and/or device includes a tissue engagement portion or anchor (e.g., a helical anchor, screw, dart, staple, hook, clasp, clip, clamp, multiple arms, multiple gripping members, two paddles, a clasp arm and a paddle arm, a gripping member and a paddle, etc.).

In some implementations, the tissue engagement portion or anchor includes a first arm (e.g., a clip arm, a clasp arm, a paddle, etc.) and a second arm (e.g., a clip arm, a clasp arm, a paddle, etc.) configured such that the first arm and the second arm can close or be moved closer together to engage and/or capture tissue (e.g., a leaflet of a native valve, membrane, lining, muscle, etc.) in the tissue engagement portion or anchor.

In some implementations, at least one of the first arm and the second arm is movable to form a capture region therebetween for capturing the tissue (e.g., leaflet, etc.) and/or moveable to bring the first arm and the second arm closer together.

In some implementations, two or more electrodes are coupled to the tissue engagement portion or anchor.

In some implementations, the system, apparatus, and/or device is configured such that: an electrical signal can be applied to the two or more electrodes, and a bioimpedance signal can be measured based on or in response to the applied electrical signal.

In some implementations, the bioimpedance signal providing an indication of a status (e.g., tissue capture status, tissue engagement status, etc.) of the tissue within the tissue engagement portion or anchor and/or an indication of a status of the tissue engagement portion or anchor (e.g., open, closed, etc.).

In some implementations, the two or more electrodes include a first electrode coupled to the first arm and a second electrode coupled to the second arm.

In some implementations, the first electrode is adjacent to the second electrode upon closing the tissue engagement portion or anchor.

In some implementations, the first electrode includes an electrode plate covering a majority of the first arm and the second electrode includes an electrode plate covering a majority of the second arm.

In some implementations, the two or more electrodes include a first electrode coupled to the first arm and a second electrode coupled to the first arm.

In some implementations, the first electrode is separated from the second electrode by a gap.

In some implementations, the first electrode and the second electrode include electrode strips parallel to a length of the first arm.

In some implementations, the first electrode and the second electrode include electrode strips parallel to a width of the first arm.

In some implementations, the first electrode is positioned on the first arm at a targeted minimum tissue capture depth.

In some implementations, the second electrode is positioned on the first arm at a targeted maximum tissue capture depth.

In some implementations, the system, apparatus, and/or device includes an electrode plate coupled to the second arm.

In some implementations, the system, apparatus, and/or device includes an impedance measurement device configured to measure bioimpedance signals and to determine a tissue capture depth based measured bioimpedance signals.

In some implementations, the impedance measurement device implements an algorithm to generate an indicator of a fully captured tissue, a partially captured tissue, and/or an overly captured tissue.

In some implementations, the system, apparatus, and/or device is configured for use at a native valve to capture leaflet tissue, and the impedance measurement device implements an algorithm to generate an indicator of a fully captured leaflet, a partially captured leaflet, and/or an overly captured leaflet.

In some implementations, the impedance measurement device implements an algorithm to generate an indicator of tissue capture depth.

In some implementations, the impedance measurement device is configured to generate an indicator of a status (e.g., tissue capture status, tissue engagement status, etc.) of the tissue when the tissue engagement portion or anchor is closed.

In some implementations, the impedance measurement device is configured to generate an indicator of capture status when the tissue engagement portion or anchor is open.

In some implementations, a system (e.g., a measurement system, detection system, bioimpedance signal measurement system, etc.) can comprise a device including a tissue engagement portion comprising a first surface and a second surface. In some implementations, the tissue engagement portion is configured such that the first surface and the second surface can close or be moved closer together to capture tissue in the tissue engagement portion. In some implementations, at least one of the first surface and the second surface is movable to form a capture region between the first surface and the second surface for capturing the tissue.

In some implementations, two or more electrodes are coupled to the tissue engagement portion.

In some implementations, the system includes an impedance measurement device. In some implementations, the impedance measurement device comprises a power supply and an electrical sensor. The power supply can be configured to apply an electrical signal to the two or more electrodes.

In some implementations, the impedance measurement device is configured to measure a bioimpedance signal using the electrical sensor.

In some implementations, the bioimpedance signal is responsive to the applied electrical signal.

In some implementations, the bioimpedance signal provides an indication of a status of the tissue within and/or proximate the tissue engagement portion.

In some implementations, the two or more electrodes are coupled to one or more anchors of the device. In some implementations, the two or more electrodes are coupled to one or more clasps of the device.

In some implementations, the impedance measurement device is configured to measure electrical characteristics from the two or more electrodes to determine the relative location of a clasp of the device and anatomy, tissue, etc. (e.g., anatomy or tissue that the device is proximate and/or in contact with, etc.).

In some implementations, the electrical characteristics include a peak-to-peak amplitude of oscillations of the bioimpedance signal. In some implementations, the electrical characteristics include an average value of a magnitude of the bioimpedance signal.

In some implementations, the system is configured to determine that the two or more electrodes are in blood and/or are not in contact with tissue based at least in part on the bioimpedance signal. In some implementations, the system is further configured to determine that the two or more electrodes are contacting targeted tissue based at least in part on the bioimpedance signal.

In some implementations, the system is configured to differentiate tissue types based at least in part on the bioimpedance signal.

In some implementations, the system is configured to determine that the two or more electrodes are transitioning from being primarily in contact with blood (and/or not in contact with tissue) to being in contact (e.g., partially in contact, primarily in contact, fully in contact, etc.) with tissue based at least in part on the bioimpedance signal.

In some implementations, the system is configured to determine that the two or more electrodes are transitioning from being partially or primarily in contact with tissue to being primarily in contact with blood (and/or not in contact with tissue) based at least in part on the bioimpedance signal.

In some implementations, the impedance measurement device comprises (e.g., on a non-transitory, computer readable medium) a signal processing algorithm capable of indicating a status of the device.

In some implementations, the impedance measurement device implements and/or executes a signal processing algorithm to indicate a status of the device.

In some implementations, the status of the device includes full capture of a leaflet, under capture of a leaflet, over capture of a leaflet, and a relative position of a leaflet in a clasp of the device.

In some implementations, the system includes a display to display a derived indicator to a user, the derived indicator indicative of a status of the device.

In some implementations, a system (e.g., a bioimpedance-based feedback system, bioimpedance system, feedback system, etc.) is configured to measure bioimpedance signals, determine tissue status (e.g., capture status, insertion status, etc.) with respect to a device (e.g., an implantable device, a treatment device, a repair device, etc.), and/or to display or otherwise provide indicators associated with the determined status. The system can employ any process, procedure, algorithm, or method described herein for measuring bioimpedance and determining tissue status with respect to an implant.

In some implementations, the system includes hardware, software, and/or firmware components for bioimpedance-based feedback. In some implementations, the system includes one or more of a data store, one or more processors, a measurement module, a capture module, and an indicator module.

In some implementations, the system comprises one or more computing devices (e.g., a single computing device, multiple computing devices, a distributed computing environment, a virtual device residing in a public or private computing cloud, etc.).

In some implementations, the system includes a measurement module to acquire or receive electrical signals from electrical components (e.g., sensors, electrodes, etc., such as any of the electrodes, sensors, arrays, etc. described anywhere herein). In some implementations, the electrical signals correspond to bioimpedance signals and can also correspond to resistance, capacitance, voltage, current, components of impedance, and the like. In some implementations, the measurement module is configured to determine an impedance value based on the acquired bioimpedance signals.

In some implementations, the system includes a status module (e.g., a capture module, deployment module, etc.) to determine a status (e.g., of a system, apparatus, and/or device) based on the bioimpedance measurements by the measurement module.

In some implementations, the bioimpedance measurements (as well as resistance, inductance, capacitance, voltage, and/or current readings) measured by the measurement module are different based on the anatomy or anatomies that indicator electrodes are near or in contact with. In some implementations, electrical characteristics measured by the measurement module, e.g., bioimpedance signals, can be used to determine the relative locations of a clasp, anchor, other device components, etc. and anatomy (e.g., tissue, etc.) or blood that a device associated with the system is proximate and/or in contact with.

In some implementations, the algorithms, methods, steps, processes, etc. herein can be stored on a non-transitory computer readable medium. In some implementation, the algorithms, methods, steps, processes, etc. herein can be implemented in a status module to determine a status (e.g., of a device, of tissue, etc.) based on the measurements acquired by the measurement module.

In some implementations, the system includes the indicator module to indicate results from the status module.

In some implementations, the system includes a data store configured to store configuration data, measurement data, analysis parameters, control commands, databases, algorithms, executable instructions (e.g., instructions for one or more processors), and the like.

In some implementations, the system includes one or more processors that are configured to control operation of one or more of the measurement module, the capture module, the indicator module, and/or the data store. In some implementations, the one or more processors implement, execute, and/or utilize the software modules, hardware components, and/or firmware elements configured to provide bioimpedance-based feedback.

In some implementations, a non-transitory computer readable medium is provided that includes computer executable instructions to cause one or more processors to perform any of the algorithms, procedures, processes, or methods described herein.

Any of the above method(s) and any methods of using the systems, assemblies, apparatuses, devices, etc. above or otherwise herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the disclosed subject matter are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. It is appreciated that these drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings.

FIG. 1 illustrates a cutaway view of the human heart in a diastolic phase.

FIG. 2 illustrates a cutaway view of the human heart in a systolic phase.

FIG. 3 illustrates a cutaway view of the human heart in a systolic phase showing valve regurgitation.

FIG. 4 is the cutaway view of FIG. 3 annotated to illustrate a natural shape of mitral valve leaflets in the systolic phase.

FIG. 5 illustrates a healthy mitral valve with the leaflets closed as viewed from an atrial side of the mitral valve.

FIG. 6 illustrates a dysfunctional mitral valve with a visible gap between the leaflets as viewed from an atrial side of the mitral valve.

FIG. 7 illustrates a tricuspid valve viewed from an atrial side of the tricuspid valve.

FIGS. 8, 9, 10, 11, 12, 13, and 14 show an example of a device or implant, in various stages of deployment.

FIG. 15 shows an example of a device or implant that is similar to the device illustrated by FIGS. 8-14, but where the paddles are independently controllable.

FIGS. 16, 17, 18, 19, 20, and 21 show the example device or implant of FIGS. 8-14 being delivered and implanted within a native valve.

FIG. 22 shows a perspective view of an example device or implant in a closed position.

FIG. 23 shows a front view of the example device or implant of FIG. 22.

FIG. 24 shows a side view of the example device or implant of FIG. 22.

FIG. 25 shows a front view of the example device or implant of FIG. 22 with a cover covering the paddles and a coaptation element or spacer.

FIG. 26 shows a top perspective view of the example device or implant of FIG. 22 in an open position.

FIG. 27 shows a bottom perspective view of the example device or implant of FIG. 22 in an open position.

FIG. 28 shows an example clasp useable in a device or implant.

FIG. 29 shows a portion of native valve tissue grasped by a clasp.

FIG. 30 shows a side view of an example device or implant in a partially open position with clasps in a closed position.

FIG. 31 shows a side view of an example device or implant in a partially open position with clasps in an open position.

FIG. 32 shows a side view of an example device or implant in a half-open position with clasps in a closed position.

FIG. 33 shows a side view of an example device or implant in a half-open position with clasps in an open position.

FIG. 34 shows a side view of an example device or implant in a three-quarters-open position with clasps in a closed position.

FIG. 35 shows a side view of an example device or implant in a three-quarters-open position with clasps in an open position.

FIG. 36 shows a side view of an example device in a fully open or full bailout position with clasps in a closed position.

FIG. 37 shows a side view of an example device in a fully open or full bailout position with clasps in an open position.

FIGS. 38, 39, 40, 41, 42, 43, 44, 45, 46, 47, 48, and 49 show the example device or implant of FIGS. 30-38, including a cover, being delivered and implanted within a native valve.

FIGS. 50A, 50B, and 50C illustrate an example system, apparatus, and/or device that can incorporate one or more of the concepts of the present application.

FIGS. 51A, 51B, 51C, 51D, and 51E illustrate examples of systems and/or devices that can incorporate one or more of the concepts of the present application.

FIGS. 52A, 52B, and 52C illustrate example clasps having two or more electrodes.

FIGS. 53A, 53B, 53C, 53D, 53E, and 53F illustrate clasps having various example electrode configurations.

FIG. 54 illustrates example bioimpedance signals from a clasp having two or more electrodes to provide bioimpedance-based feedback.

FIGS. 55 and 56 illustrate example bioimpedance signals from the clasp of FIG. 53B.

FIGS. 57 and 58 illustrate example bioimpedance signals from the clasp of FIG. 53C.

FIGS. 59A and 59B illustrate an example clasp that is configured similar to the clasp of FIGS. 22-37 with an optional cover over the clasp, the clasp including a combination of an electrode plate on one arm and electrode strips on the other arm.

FIGS. 60A and 60B illustrate a clasp that is configured similar to the clasp of FIGS. 22-37 with an optional cover over the clasp, the clasp including electrode strips on an arm of the clasp.

FIG. 60C illustrates an example clasp similar to the clasp of FIG. 60B without a cover, the clasp including electrode strips on an arm of the clasp.

FIGS. 61A and 61B illustrate the real and imaginary portions of bioimpedance signals of the clasp of FIGS. 60A-60C for various capture states of a leaflet.

FIGS. 62A and 62B illustrate an implementation of a tissue engagement portion or clasp similar to the clasp of FIGS. 60A-60C with the electrode strips offset from an edge of an arm by a prescribed distance.

FIG. 62C illustrates bioimpedance signals of the clasp of FIGS. 62A and 62B for various capture states of a leaflet.

FIG. 62D illustrates an example proximal end of a delivery system that includes an indicator panel.

FIGS. 63A and 63B illustrates an example device with clasps each having a first electrode positioned on a first arm and a second electrode positioned on a second arm of the clasps, the electrodes configured to provide bioimpedance signals corresponding to different dies of a leaflet or other tissue.

FIG. 64 illustrates an example device with tissue engagement portions or clasps having electrodes similar to the device with clasps of FIG. 53A, with the addition of a reference electrode implemented on the device.

FIG. 65 illustrates an example of the device of FIGS. 22-37 with the addition of flexible electrodes that protrude away from the device.

FIGS. 66A and 66B illustrate example electrode arrays that reduce the number of electrical leads required to enable the electrical leads to fit into small-lumen catheters.

FIG. 67A illustrates an example of a bioimpedance signal with oscillations corresponding to diastole and systole of the heart.

FIG. 67B illustrates an example of a bioimpedance signal as a delivery device implants an annuloplasty ring in an annulus.

FIG. 68 illustrates an example bioimpedance signal measurement system.

FIG. 69 illustrates a portion of a flexible PCB with a stress concentration point in the PCB.

FIG. 70 illustrates a portion of a flexible PCB with a Y-shaped protrusion extending from one end of the PCB.

FIG. 71 illustrates a portion of a flexible PCB with a round protrusion extending from a body of the PCB.

FIG. 72 illustrates a portion of a flexible PCB with side indents to facilitate securing the suture over the PCB and to the device to secure the PCB to the device.

FIG. 73 illustrates a portion of a flexible PCB forming a circular hole with a relief.

FIG. 74 illustrates a portion of a flexible PCB that forms a pair of bi-directional tongues.

FIGS. 75A, 75B, and 75C illustrate a removable PCB that is configured to be pulled through the barbs of a device to remove the PCB from the device.

FIGS. 76A, 76B, and 76C illustrate another removable PCB that is configured to be pulled and exit through a side of a clasp, around barbs of a device to remove the PCB from the device.

FIGS. 77A, 77B, and 77C illustrate another removable PCB that is configured to be split apart when pulled so that half exits through one side of a clasp and the other half exits through the other side of the clasp, each half exiting the clasp around barbs of a device to remove the PCB from the device.

FIG. 78 illustrates an electrode that is removable from a device, the electrode being coupled to wires extending from the electrode towards an actuation element.

FIGS. 79A and 79B illustrate spring pin electrical connectors configured to extend to a distal end of a delivery system to provide electrical connection with wires of an electrode coupled to the device.

FIGS. 80A and 80B illustrate using radial forces via fingers of a delivery system to couple wires coming from the delivery system to electrical leads coupled to an electrode of the device.

FIGS. 81A and 81B illustrate the use of a tube to enable releasable electrical contact between wires and electrical leads.

FIGS. 82A and 82B illustrate a coil crimp configured to provide releasable electrical contact between wires and electrical leads.

FIGS. 83A and 83B illustrate a coil connection socket configured to provide releasable electrical contact between wires and electrical leads.

FIGS. 84A, 84B, 84C, and 84D illustrate an example disc crimp configured to provide releasable electrical connection between wires and electrical leads.

FIGS. 85A, 85B, 85C, 85D, 85E, and 85F illustrate examples of heat-activated electrical connectors to provide releasable electrical connections between wires and electrical leads.

FIG. 86 illustrates a block diagram of an example bioimpedance-based feedback system.

### DETAILED DESCRIPTION

The following description refers to the accompanying drawings, which illustrate example implementations of the present disclosure. Other implementations having different structures and operation do not depart from the scope of the present disclosure.

### Overview

Disclosed herein are devices and methods that use bioimpedance or bioimpedance-based feedback to provide useful information during a medical procedure. Bioimpedance or bioimpedance-based feedback includes measuring or acquiring electrical signals that include a bioimpedance signal (e.g., a signal that is indicative of bioimpedance). The bioimpedance signal can be used to determine the position and/or status of a device (e.g., of an implantable device, of a treatment device, of a delivery device, etc.) or portion thereof (e.g., such as a clasp, valve, anchor, or the like) relative to tissue or other portions of a body. The bioimpedance signal can be analyzed and converted into information presented to a clinician (e.g., words, images, symbols, colors, etc. displayed on a display, sounds, lights, etc.) to indicate a position and/or status of the device (e.g., a position and/or status of anchoring elements of an implant, etc.).

Bioimpedance is related to electrical properties of tissue within the body (or other biomaterials). Bioimpedance is a measure of how well the tissue impedes electric current flow. Fat has high resistivity; blood has lower resistivity. At a given current applied to the tissue, a low impedance will correspond to a low voltage and vice versa. Tissue includes cells and membranes, and membranes are thin with high resistivity and behave electrically as capacitors. By using high measuring frequencies, the current passes through these capacitors, and the resulting signal depends on tissue and liquids both inside and outside the cells. At low frequencies, however, the membranes impede current flow, and the results are dependent only on liquids outside the cells. The magnitude and phase of the impedance Z is given by:

$$\vert Z \vert = \sqrt{{R}^{2} + {( {X}_{L} - {X}_{C} )}^{2}} \\ {θ}_{Z} = {tan}^{- 1} \frac{X}{R}$$

Where R is the resistance, X_L is the inductive reactance, X_C is the capacitive reactance, R is the total resistance, and X is the total reactance. The impedance can also be expressed using real and imaginary components as: Z=R+jX.

In some implementations, the systems and/or apparatuses herein comprise devices (e.g., treatment devices, repair devices, implantable devices, etc.) or portions of devices including electrodes. In some implementations, the systems and/or apparatuses herein comprise delivery systems and/or devices or portions thereof including electrodes (e.g., catheters with electrodes, etc.).

Electrical power, e.g., in the form of alternating current, direct current, etc., can be provided to the electrodes and electrical signals measured (e.g., voltage, current, changes in voltage, changes in current, etc.). Bioimpedance signals, which form part of the measured electrical signals (or can be determined from the electrical signals), can be used to draw conclusions or estimates related to the system/device (e.g., related to the status of the device or an anchoring portion of the device, etc.).

In some implementations, where electrodes are implemented on a clasp, clamp, clip, gripping portion, anchor, etc. of a device (e.g., an implantable device, a treatment device, a repair device, etc.), the bioimpedance signal can be correlated to how much tissue is within the clasp, clamp, clip, gripping portion, anchor, etc. of the device.

In some implementations, bioimpedance signals can also be used to monitor the depth of an anchor (e.g., a helical anchor, a tissue anchor, a screw, a dart, a staple, etc.) of a device in tissue, valve height and positioning, consecutive anchor deployment, and the like.

In some implementations, the bioimpedance signal can be analyzed and presented in real time to provide useful information to a clinician implanting an implantable device and/or while using a treatment device or repair device (even if not permanently implanted). This is one-way bioimpedance signals can be used to provide useful feedback to clinicians or medical systems regarding the status of a device or implant and/or components thereof.

In some implementations, values of a bioimpedance signal and/or changes in the bioimpedance signal can indicate a transition from being primarily in blood to contacting tissue. In some implementations, values of a bioimpedance signal and/or changes in the bioimpedance signal can indicate a transition from being in contact with a first type of tissue (e.g., a leaflet, etc.) being in contact with a second type of tissue (e.g., an annulus, heart wall, etc.).

In some implementations, the value or change in bioimpedance signal can correlate to an amount of contact with tissue the device has (e.g., the amount of leaflet in a clasp, the depth of an anchor in tissue, valve height and/or positioning, and the like).

In some implementations, the value or change in bioimpedance signal can correlate to the location/position of a delivery device (e.g., a catheter, anchor driver, hypotube, pusher, etc.) and/or whether the delivery device is in contact with tissue or different types of tissue.

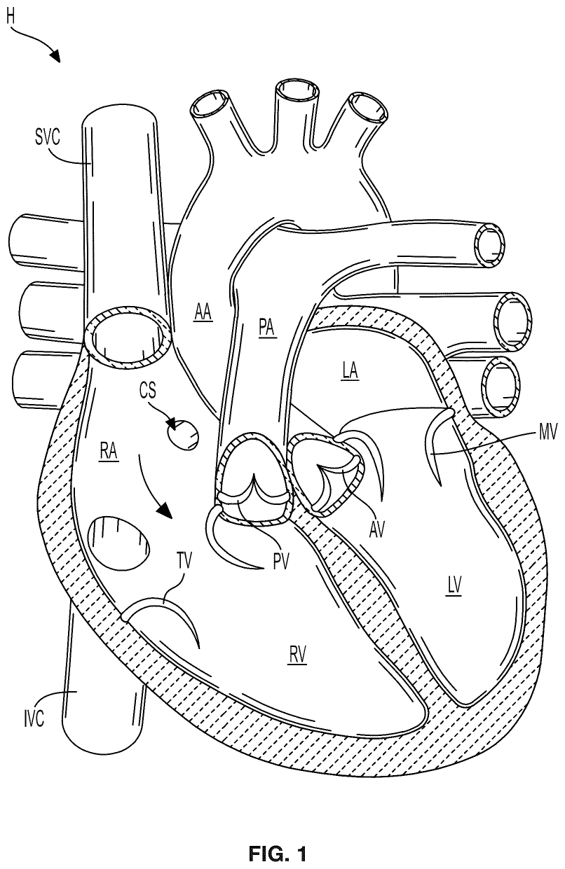

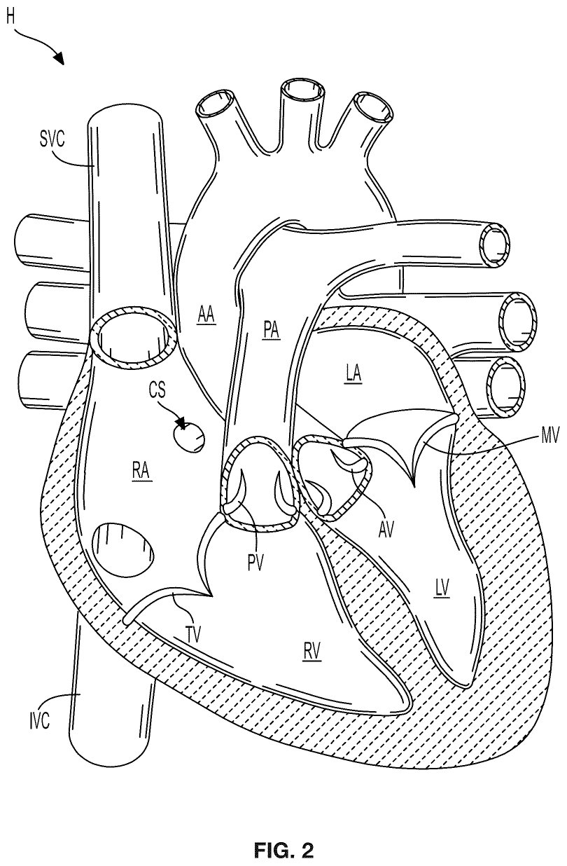

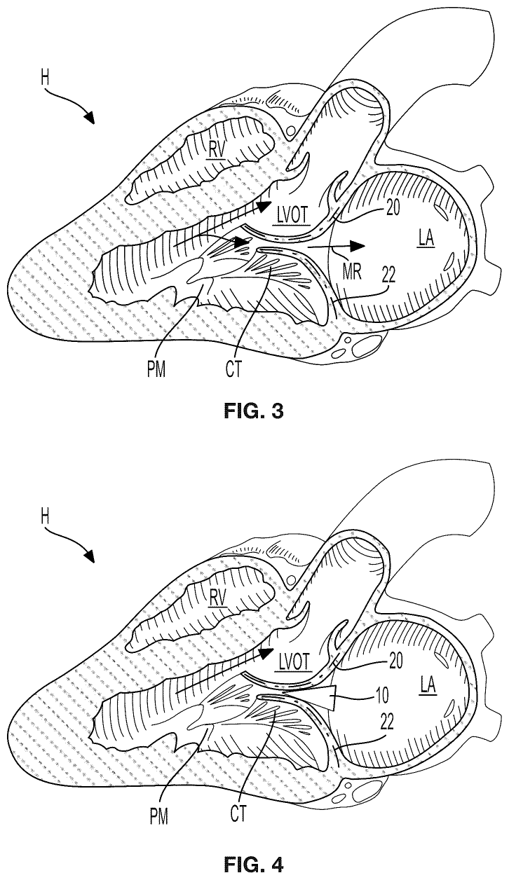

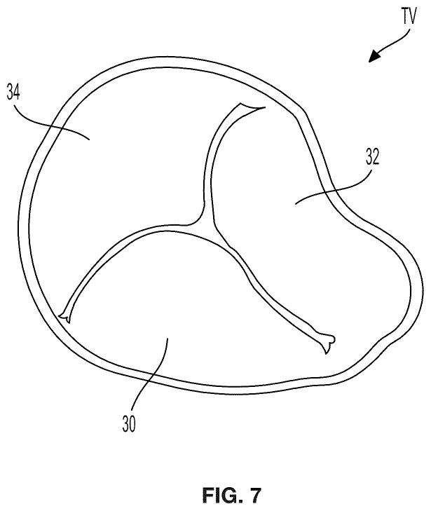

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent or inhibit blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

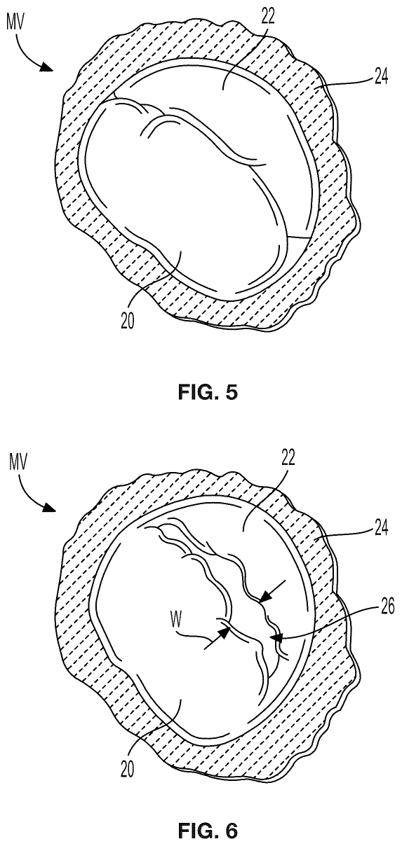

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm. As set forth above, there are several different ways that a leaflet (e.g., leaflets 20, 22 of mitral valve MV) may malfunction which can thereby lead to valvular regurgitation.

In any of the above-mentioned situations, a system, an apparatus, and/or device (e.g., a treatment system, a repair system, valve repair device, valve treatment device, implant, etc.) is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a device, valve repair device, or implant 10 is shown implanted between the leaflets 20, 22 such that regurgitation does not occur during systole (compare FIG. 3 with FIG. 4). In some implementations, the coaptation element (e.g., spacer, coaption element, gap filler, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, prevents the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to treating and/or repairing the structure of a mitral valve. However, it should be understood that the devices and concepts provided herein can be used in conjunction with procedures on any native valve (e.g., the tricuspid valve), as well as any other medical procedure implanting an implantable device and/or gripping tissue as part of a treatment and/or repair procedure (even if the device is not implanted).

### Example Devices

An example device or implant (e.g., a treatment device, a repair device, a valve repair device, an implantable device, an implantable prosthetic device, etc.) with which the concepts herein can be implemented can optionally have a coaptation element (e.g., spacer, coaption element, gap filler, etc.) and at least one anchor (e.g., one, two, three, or more). In some implementations, a device or implant can have any combination or sub-combination of the features disclosed herein without a coaptation element.

In some implementations, when included, the coaptation element (e.g., coaption element, spacer, etc.) can be configured to be positioned within the native heart valve orifice to help fill the space between the leaflets and form a more effective seal, thereby reducing or preventing or inhibiting regurgitation described above.

In some implementations, the optional coaptation element can have a structure that is impervious and/or resistant to blood flow therethrough (or otherwise reduces or inhibits blood flow) and that allows the native leaflets to close around the coaptation element during ventricular systole to block blood from flowing from the left or right ventricle back into the left or right atrium, respectively. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The device or implant can be configured to seal against two or three native valve leaflets; that is, the device can be used in the native mitral (bicuspid) and tricuspid valves.

The optional coaptation element (e.g., spacer, coaption element, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

In some implementations, the anchor (e.g., a clasp, a clip, a clamp, multiple arms, multiple gripping members, two paddles, a clasp arm and a paddle arm, a gripping member and a paddle arm, etc.) can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent to the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element, e.g., shaft, actuation wire, etc. The anchor can be configured to be positioned behind a native leaflet when used and/or implanted such that the leaflet is grasped by the anchor.

The device or implant can be configured to be used, operated, and/or implanted via a delivery system or other means for delivery. The delivery system can comprise one or more of a guide/delivery sheath, a delivery catheter, a steerable catheter, an implant catheter, tube, combinations of these, etc. The optional coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. Various example methods are more fully discussed below with respect to each implementation.

Additional information regarding these and other delivery methods that can be used with the various systems and devices herein can be found in U.S. Pat. No. 8,449,599 and U.S. Patent Application Publication Nos. 2014/0222136, 2014/0067052, 2016/0331523, PCT patent application publication No. WO2020/076898, PCT patent application No. PCT/US2022/035672, PCT patent application No. PCT/US2022/037983, PCT patent application No. PCT/US2022/050158, PCT patent application No. PCT/US2022/051232, PCT patent application No. PCT/US2022/049305, PCT patent application No. PCT/US2022/037176, and PCT patent application No. PCT/US2022/025390, each of which is incorporated herein by reference in its entirety for all purposes. These method(s) can be performed on a living animal or on a simulation, such as on a cadaver, cadaver heart, anthropomorphic ghost, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

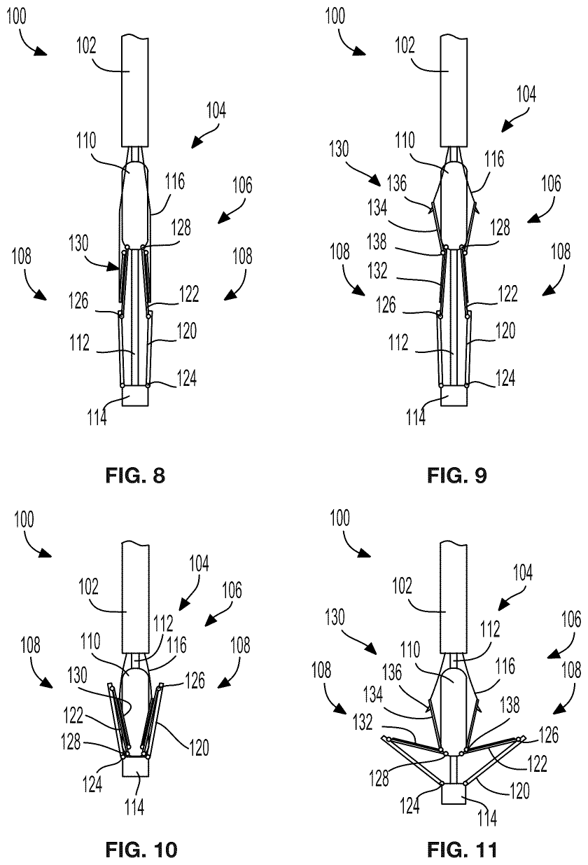

Referring now to FIGS. 8-15, a schematically illustrated example device or implant 100 (e.g., a prosthetic spacer device, valve repair device, valve treatment device, etc.) is shown in various stages of deployment. The device or implant 100 and other similar devices/implants that can be used with the various implementation, systems, and devices herein are described in more detail in PCT patent application publication Nos. WO 2018/195215, WO 2020/076898, WO 2019/139904, PCT patent application No. PCT/US2022/035672, PCT patent application No. PCT/US2022/037983, PCT patent application No. PCT/US2022/050158, PCT patent application No. PCT/US2022/051232, PCT patent application No. PCT/US2022/049305, PCT patent application No. PCT/US2022/037176, and PCT patent application No. PCT/US2022/025390 which are incorporated herein by reference in their entirety for all purposes. The device 100 (and other systems and devices herein) can include any other features for an device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited herein).

The device or implant 100 is deployed from a delivery system, delivery device, or other means for delivery 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 can include an optional coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, actuation shaft, actuation tube, etc.).

In some implementations, the anchor portion 106 includes one or more anchors 108 that are actuatable between open and closed conditions and can take a wide variety of forms, such as, for example, paddles, gripping elements, a clasp, a clip, a clamp, a clasp arm and a paddle arm, a gripping member and a paddle, etc.) or the like. Actuation of the means for actuating or actuation element 112 opens and closes the anchor portion 106 of the device 100 to grasp the native valve leaflets during implantation. The means for actuating or actuation element 112 (as well as other means for actuating and actuation elements herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and means for actuating or actuation element 112 (e.g., actuation wire, actuation shaft, etc.). These can be configured to extend through a guide catheter/sheath (e.g., a transseptal sheath, etc.). In some implementations, the means for actuating or actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element removably attaches the coaptation element 110 to the delivery system 102, either directly or indirectly, so that the means for actuating or actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members. As illustrated, in some implementations, gripping members (or tissue engagement portions) can comprise clasps 130 that include a base or fixed arm 132, a movable arm 134, optional barbs, friction-enhancing elements, or other means for securing 136 (e.g., protrusions, ridges, grooves, textured surfaces, adhesive, etc.), and a joint portion 138.

In some implementations, a fixed arm may not be used and another portion of the device (e.g., another component, surface, element, etc.) can perform functions described herein with respect to the fixed arm.

In some implementations, the fixed arms 132 are attached to the inner paddles 122. In some implementations, the fixed arms 132 are attached to the inner paddles 122 with the joint portion 138 disposed proximate a coaptation element 110. In some implementations, the clasps (e.g., barbed clasps, etc.) have flat surfaces and do not fit in a recess of the inner paddle. Rather, the flat portions of the clasps are disposed against the surface of the inner paddle 122. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional barbs, friction-enhancing elements, or means for securing 136.

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. This pinching force remains constant regardless of the position of the inner paddles 122. Optional barbs, friction-enhancing elements, or other means for securing 136 of the clasps 130 can grab, pinch, and/or pierce the native leaflets to further secure the native leaflets.

During implantation, the paddles 120, 122 can be opened and closed, for example, to grasp tissue (e.g., native leaflets, native mitral valve leaflets, native tricuspid valve leaflets, etc.) between the paddles 120, 122 and/or between the paddles 120, 122 and a coaptation element 110. The clasps 130 can be used to grasp and/or further secure the tissue by engaging the tissue with optional barbs, friction-enhancing elements, or means for securing 136 and pinching the tissue (e.g., leaflets, etc.) between the movable and fixed arms 134, 132. The optional barbs, friction-enhancing elements, or other means for securing 136 (e.g., protrusions, ridges, grooves, textured surfaces, adhesive, etc.) of the tissue engagement portions or clasps 130 increase friction with the tissue or can partially or completely puncture the tissue.

In some implementations, the actuation lines 116 can be actuated separately so that each tissue engagement portion or clasp 130 can be opened and closed separately. Separate operation allows one leaflet to be grasped at a time, or for the repositioning of a tissue engagement portion or clasp 130 on tissue (e.g., a leaflet, etc.) that was insufficiently grasped, without altering a successful grasp on the other leaflet. In some implementations, the clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the example device 100 is shown in an elongated or fully open condition for deployment from an implant delivery catheter of the delivery system 102. The device 100 is disposed at the end of the catheter in the fully open position, because the fully open position takes up the least space and allows the smallest catheter to be used (or the largest device to be used for a given catheter size). In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 are kept in a closed condition during deployment through the delivery system 102 so that the optional barbs, friction-enhancing elements, or other means for securing 136 (FIG. 9) do not catch or damage the delivery system 102 or tissue in the patient's heart.

Referring now to FIG. 9, the device 100 is shown in an elongated detangling condition, similar to FIG. 8, but with the clasps 130 in a fully open position, ranging from about 140 degrees to about 200 degrees, from about 170 degrees to about 190 degrees, or about 180 degrees between fixed and movable arms 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the means for actuating or actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open to closed position, the outer paddles 120 maintain an acute angle with the means for actuating or actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition. In some implementations, the inner paddles 122 are thinner and/or narrower than the outer paddles 120, and the connection portions 126, 128 (e.g., joints, flexible connections, etc.) connected to the inner paddles 122 can be thinner and/or more flexible. For example, this increased flexibility can allow more movement than the connection portion 124 connecting the outer paddle 120 to the cap 114. In some implementations, the outer paddles 120 are narrower than the inner paddles 122. The connection portions 126, 128 connected to the inner paddles 122 can be more flexible, for example, to allow more movement than the connection portion 124 connecting the outer paddle 120 to the cap 114. In some implementations, the inner paddles 122 can be the same or substantially the same width as the outer paddles.

Referring now to FIGS. 11-13, the device 100 is shown in a partially open, grasp-ready condition. To transition from the fully closed to the partially open condition, the means for actuating or actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the targeted tissue or leaflets can be grasped. In some implementations, the pair of inner and outer paddles 122, 120 are moved in unison, rather than independently, by a single means for actuating or single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. For example, the device 100 can have two actuation elements and two independent caps (or other attachment portions), such that one independent actuation element (e.g., wire, shaft, etc.) and cap (or other attachment portion) are used to control one paddle, and the other independent actuation element and cap (or other attachment portion) are used to control the other paddle.

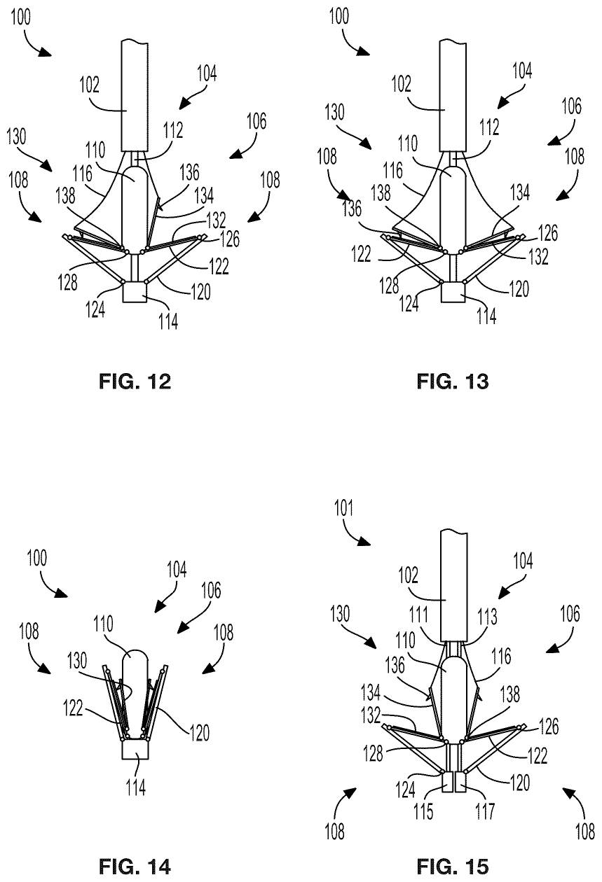

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system or means for delivery 102 and means for actuating or actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device 100 illustrated by FIG. 11, except the device 101 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the means for actuating or actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the means for actuating or actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single means for actuating or actuation element 112.

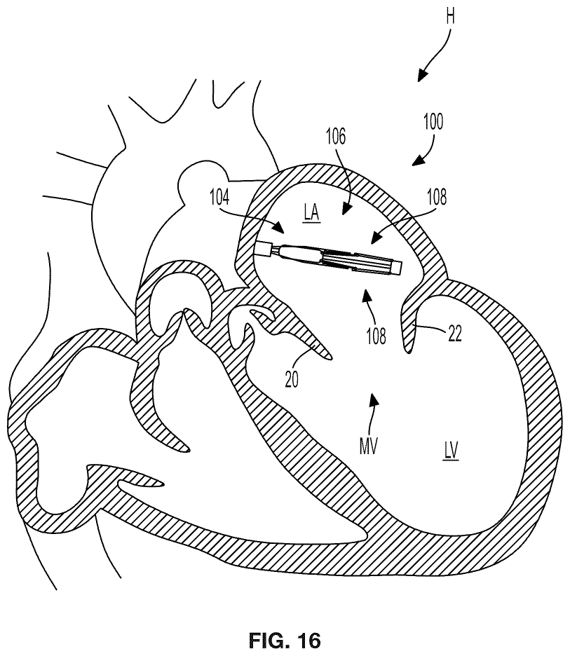

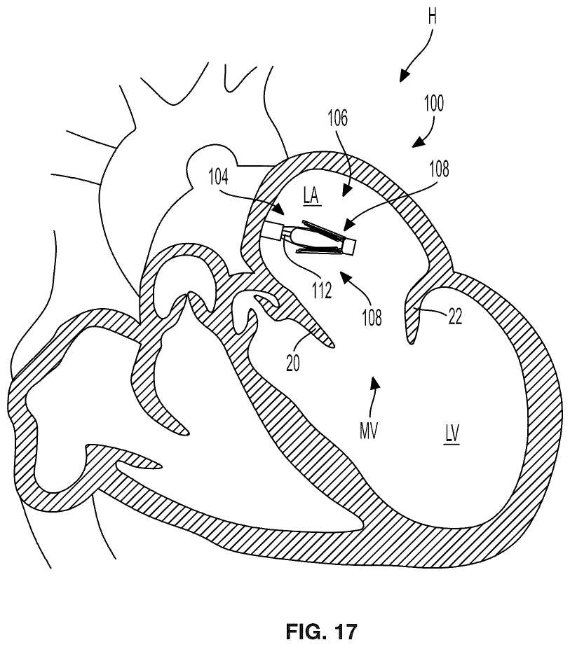

Referring now to FIGS. 16-21, the device 100 of FIGS. 8-14 is shown being delivered and implanted within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The means for actuating or actuation element 112 is then retracted to move the implant/device into the fully closed condition shown in FIG. 17.

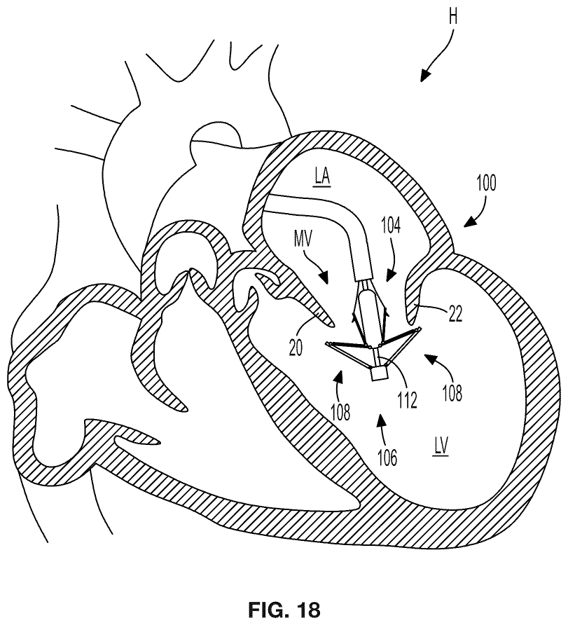

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant/device as illustrated by FIG. 18.

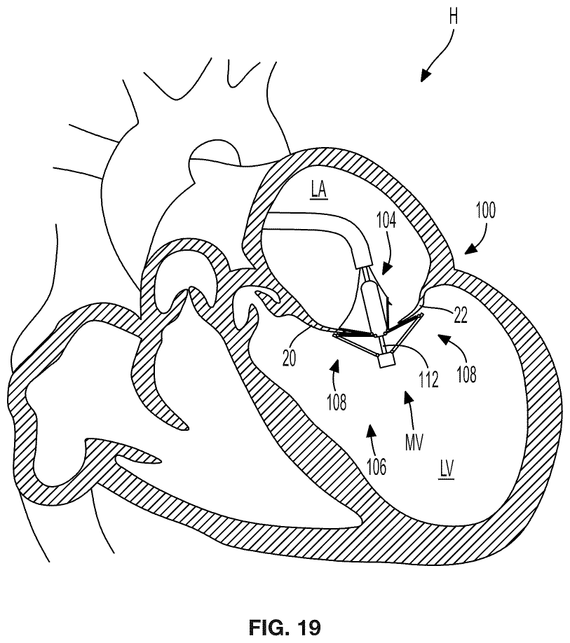

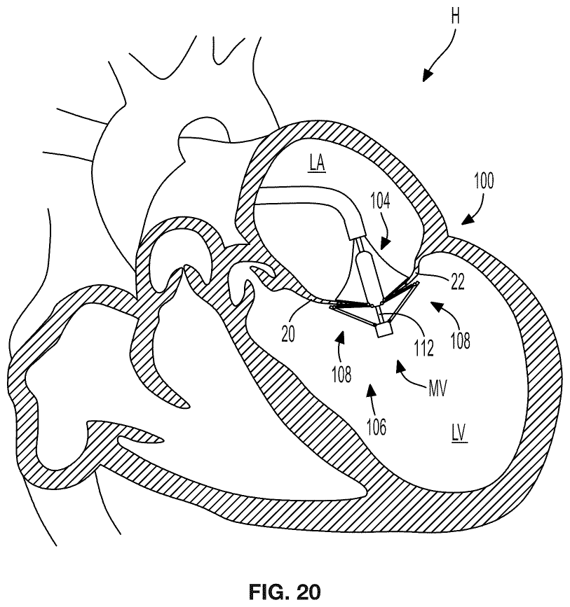

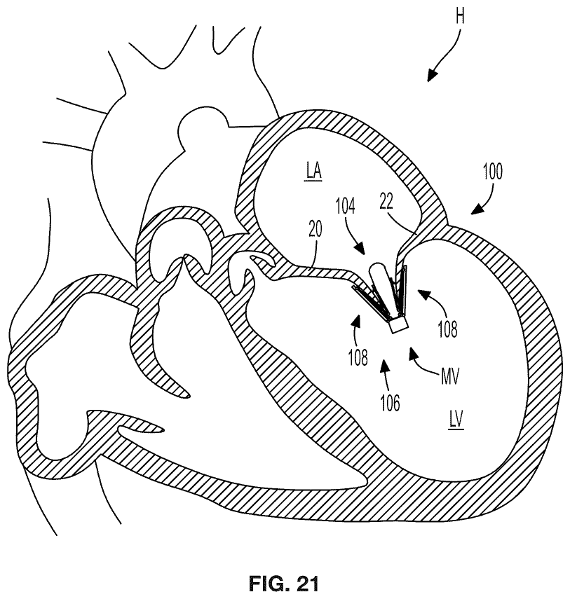

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), means for actuating or actuation element 112 and actuation lines 116 are then retracted and the device or implant 100 is fully closed and deployed in the native mitral valve MV.

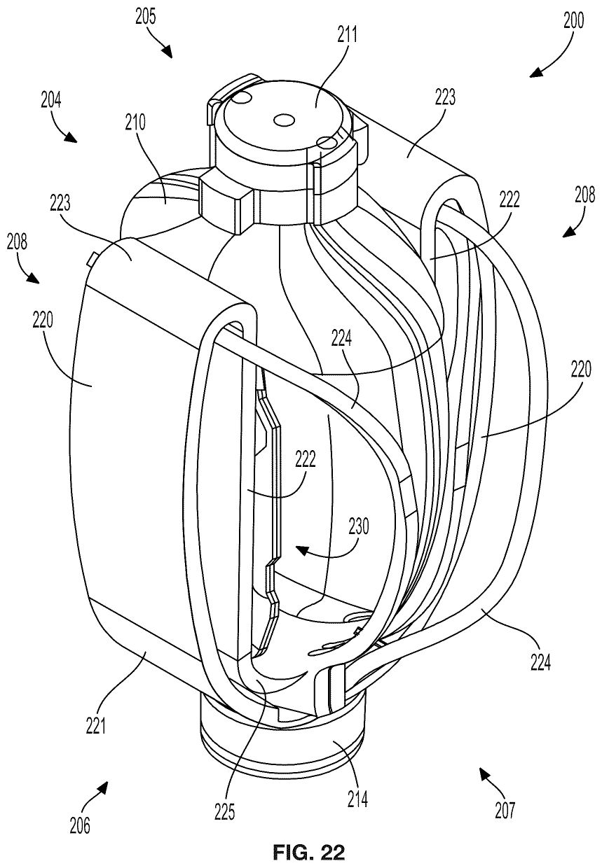

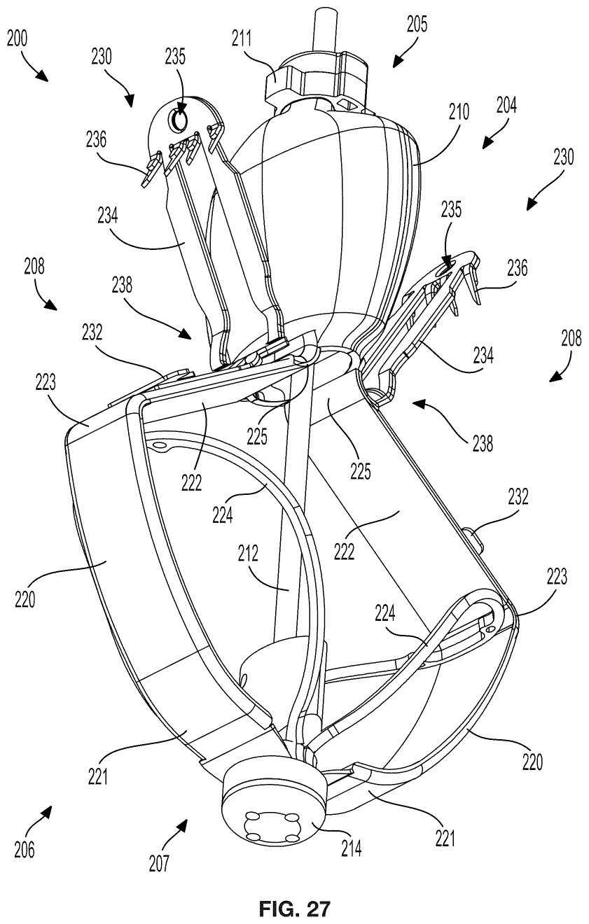

Referring now to FIGS. 22-27, an example of a device or implant 200 is shown. The devices herein, including device 100 that is schematically illustrated in FIGS. 8-14, can be configured the same as or similar to device 200. The device 200 can include any other features for a device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

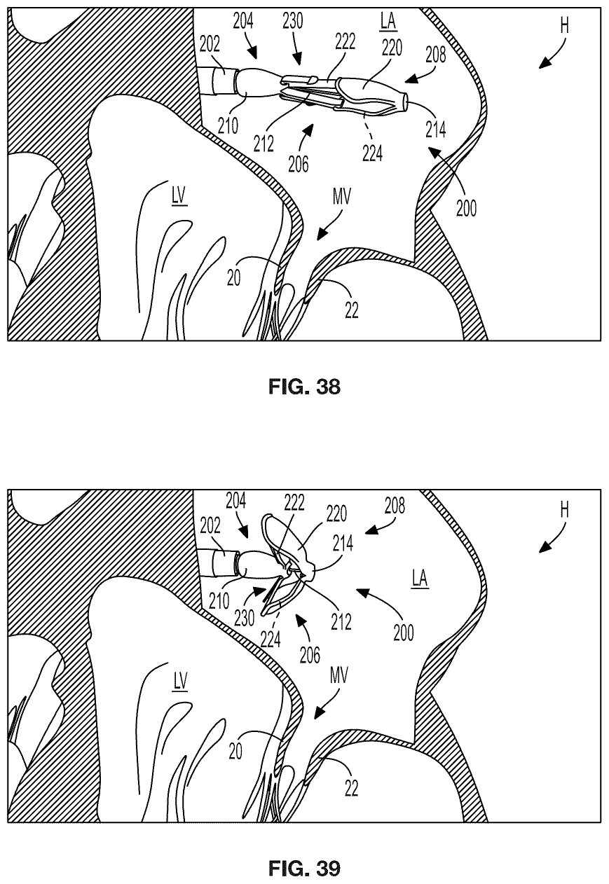

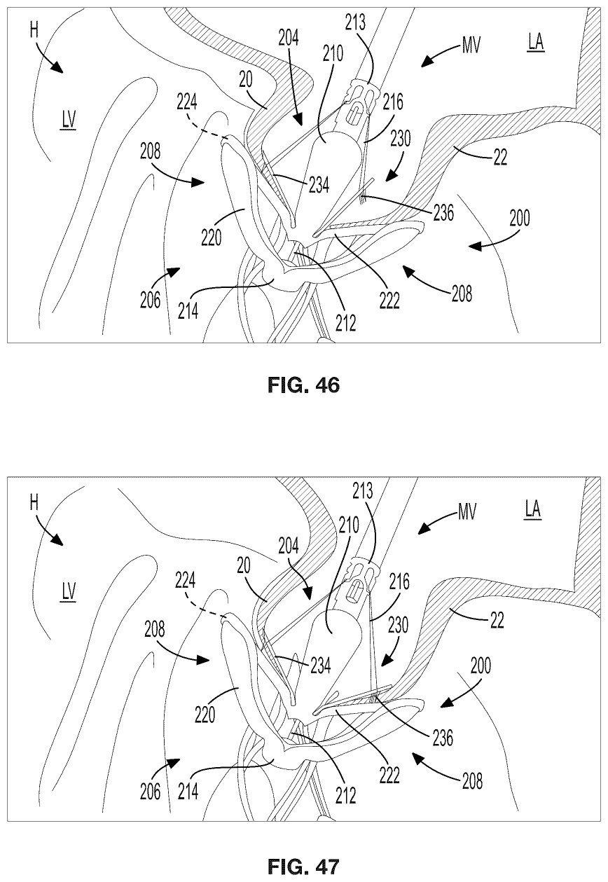

In some implementations, the device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and gripping elements or clasps 230. In some implementations, the attachment portion 205 includes a first or proximal component or collar 211 (or other attachment element, extension, ring, etc.) for engaging with a capture mechanism 213 (FIGS. 43-49) of a delivery system 202 (FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the device or implant 200. In some implementations, the actuation element 212 extends through the capture mechanism 213, proximal component or collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

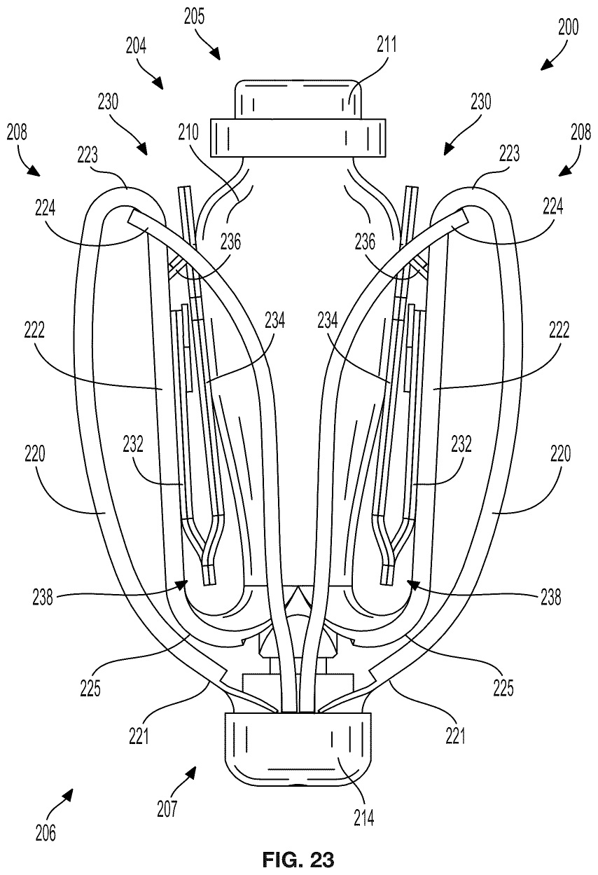

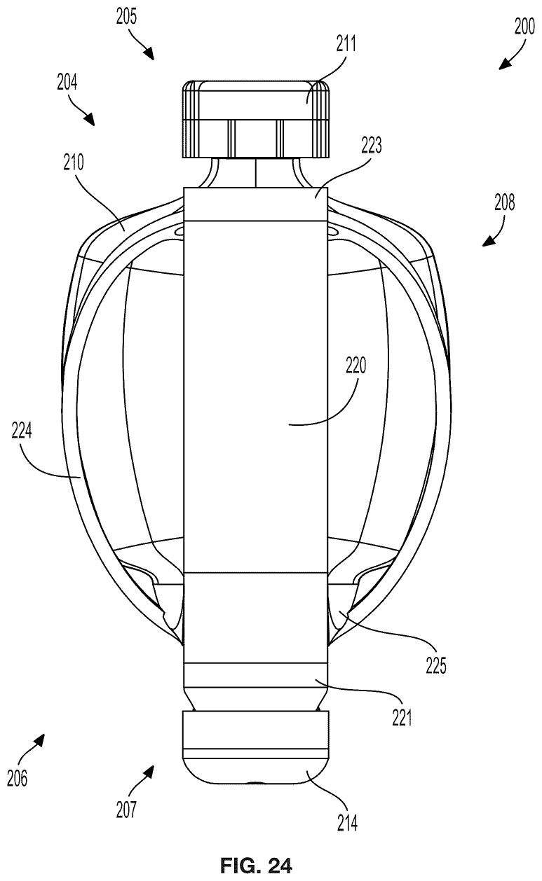

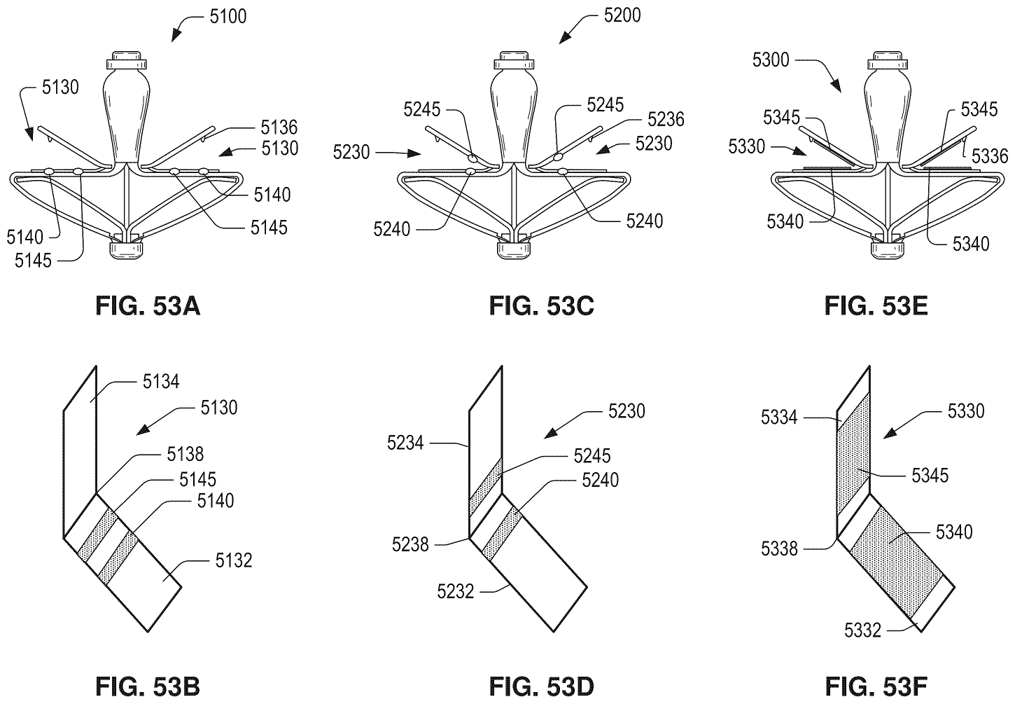

The coaptation element 210 extends from the proximal component or collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 53A) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed arm 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 53A. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 53A, the increased surface area of the paddle frames 224 can also allow the native leaflets to be clamped to the device or implant 200, such that the native leaflets coapt entirely around the coaptation member or coaptation element 210. This can, for example, improve sealing of the native leaflets 20, 22 and thus prevent or further reduce mitral regurgitation.

In some implementations, the clasps comprise a movable arm coupled to the anchors. In some implementations, the clasps 230 include a base or fixed arm 232, a movable arm 234, optional barbs 236, and a joint portion 238. In some implementations, the fixed arms 232 are attached to the inner paddles 222, with the joint portion 238 disposed proximate the coaptation element 210. The joint portion 238 is spring-loaded so that the fixed and movable arms 232, 234 are biased toward each other when the clasp 230 is in a closed condition. In some implementations, the clasps 230 include friction-enhancing elements or means for securing, such as optional barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the movable arms 234 are opened to open the clasps 230 and expose the optional barbs or other friction-enhancing elements 236. The clasps 230 are opened by applying tension to actuation lines 216 (e.g., as shown in FIGS. 43-48) attached to holes 235 in the movable arms 234, thereby causing the movable arms 234 to articulate, pivot, and/or flex on the joint portions 238.

Referring now to FIG. 29, a close-up view of one of the leaflets 20, 22 grasped by a tissue engaging portion such as clasp 230 is shown. The leaflet 20, 22 is shown grasped between the movable and fixed arms 234, 232 of the clasp 230. The tissue of the leaflet 20, 22 is not pierced by the optional barbs or friction-enhancing elements 236, though in some implementations the optional barbs 236 can partially or fully pierce through the leaflet 20, 22. The angle and height of the optional barbs or friction-enhancing elements 236 relative to the movable arm 234 helps to secure the leaflet 20, 22 within the clasp 230. In particular, a force pulling the device off of the native leaflet 20, 22 will encourage the optional barbs or friction-enhancing elements 236 to further engage the tissue, thereby ensuring better retention. Retention of the leaflet 20, 22 in the clasp 230 is further improved by the position of fixed arm 232 near the optional barbs/friction-enhancing elements 236 when the clasp 230 is closed. In this arrangement, the tissue is formed by the fixed arms 232 and the movable arms 234 and the optional barbs/friction-enhancing elements 236 into an S-shaped torturous path. Thus, forces pulling the leaflet 20, 22 away from the clasp 230 will encourage the tissue to further engage the optional barbs/friction-enhancing elements 236 before the leaflets 20, 22 can escape. For example, leaflet tension during diastole can encourage the optional barbs 236 to pull toward the end portion of the leaflet 20, 22. Thus, the S-shaped path can utilize the leaflet tension during diastole to more tightly engage the leaflets 20, 22 with the optional barbs/friction-enhancing elements 236.

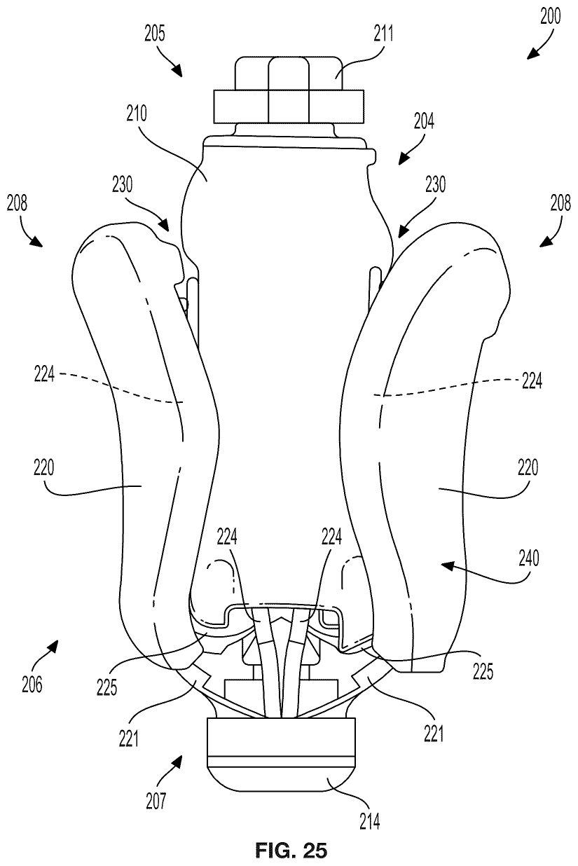

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the device or implant 200.

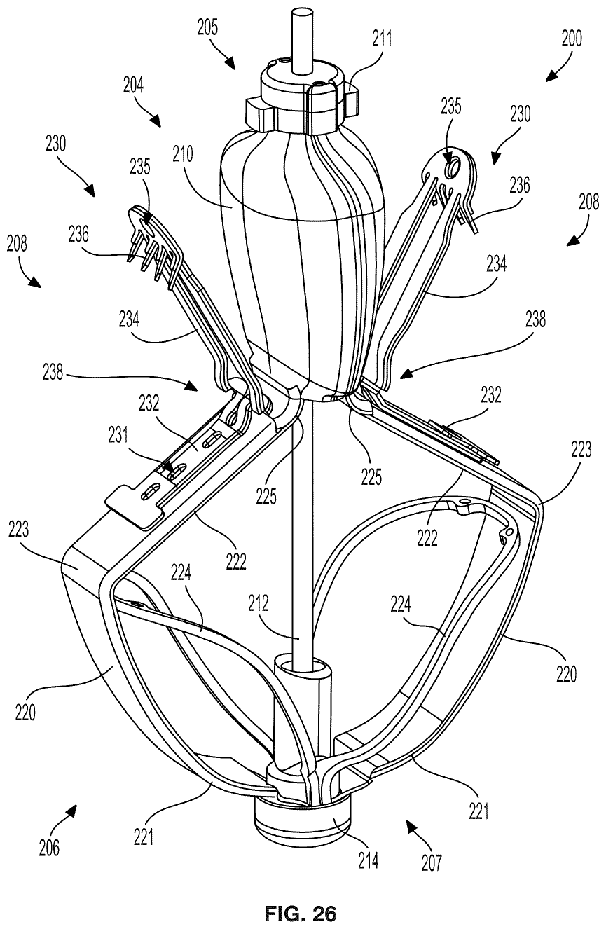

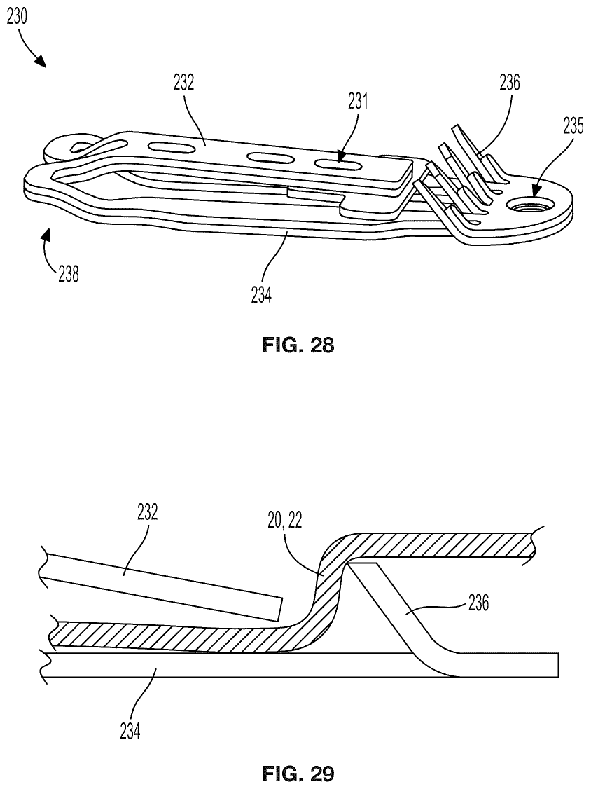

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal component or collar 211 (or other attachment element, extension, ring, etc.) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 101 illustrated in FIG. 15).

In some implementations, the clasps 230 further secure the native leaflets 20, 22 by engaging the leaflets 20, 22 with optional barbs and/or other friction-enhancing elements 236 and/or pinching the leaflets 20, 22 between the movable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to more securely grip the leaflets 20, 22 when the device 200 is closed (e.g., as can be seen in FIG. 49). The curved shape and rounded edges of the outer paddle 220 also prohibits or inhibits tearing of the leaflet tissue.

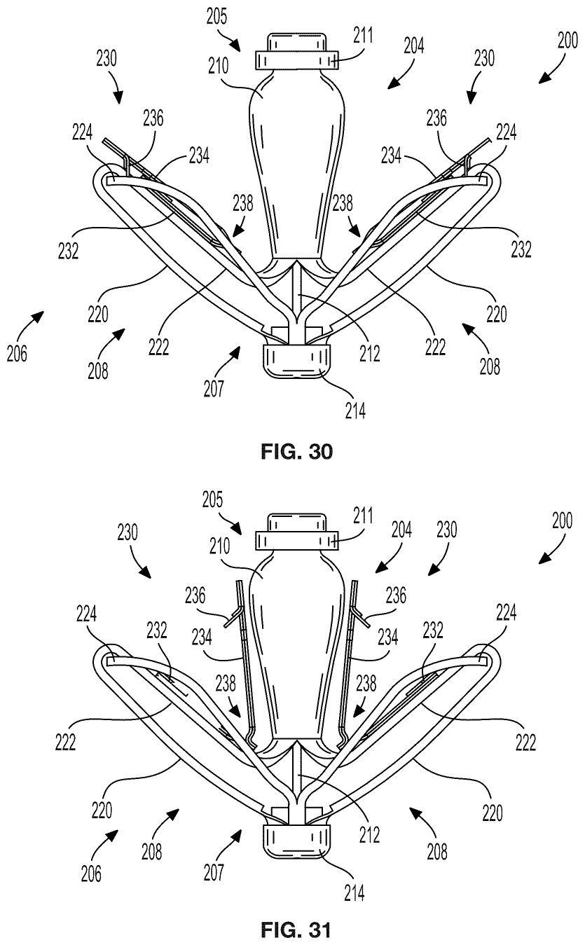

Referring now to FIGS. 30-37, the device or implant 200 described above is shown in various positions and configurations ranging from partially open to fully open. The paddles 220, 222 of the device 200 transition between each of the positions shown in FIGS. 30-37 from the closed position shown in FIGS. 22-25 by extension of the actuation element 212 from a fully retracted to a fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal component or collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames 224 all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and movable arms 232, 234 of the clasp 230 is limited to the extent that the inner paddle 222 has spread away from the coaptation element 210.

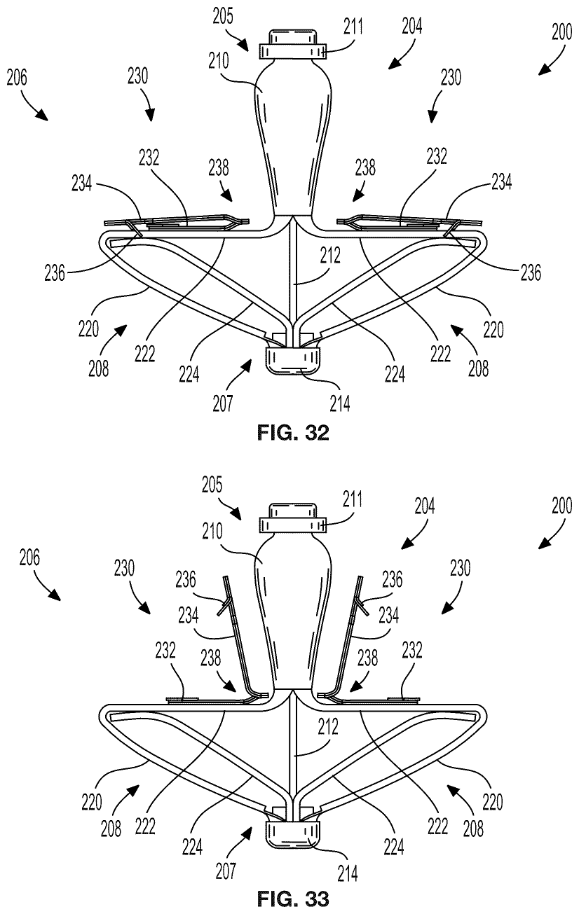

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and movable arms 232, 234.

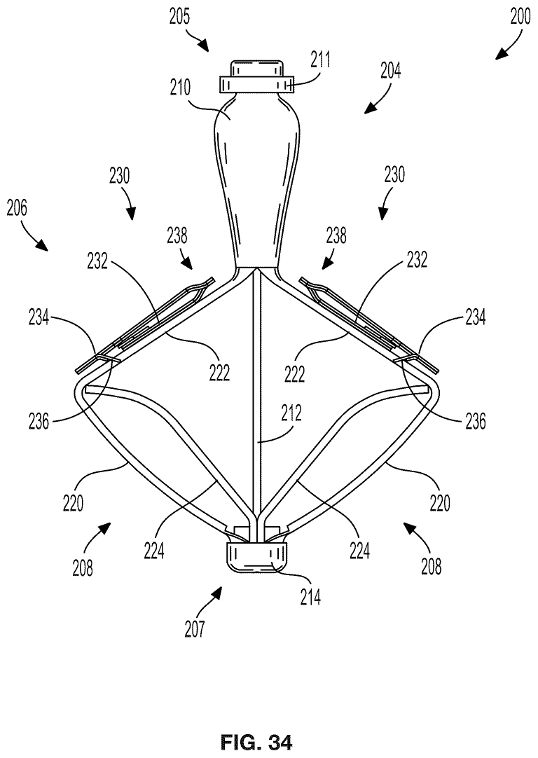

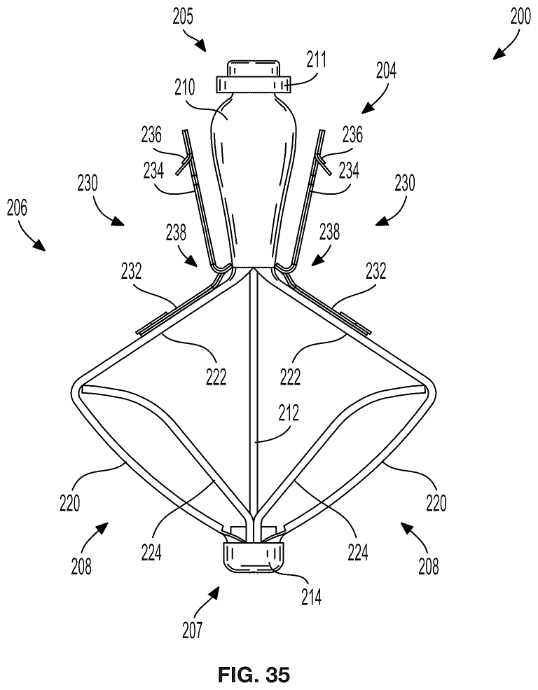

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and movable arms 232, 234.

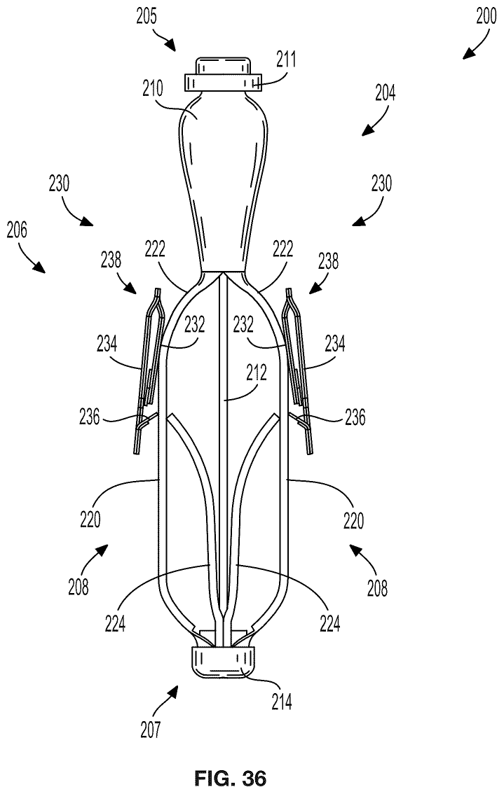

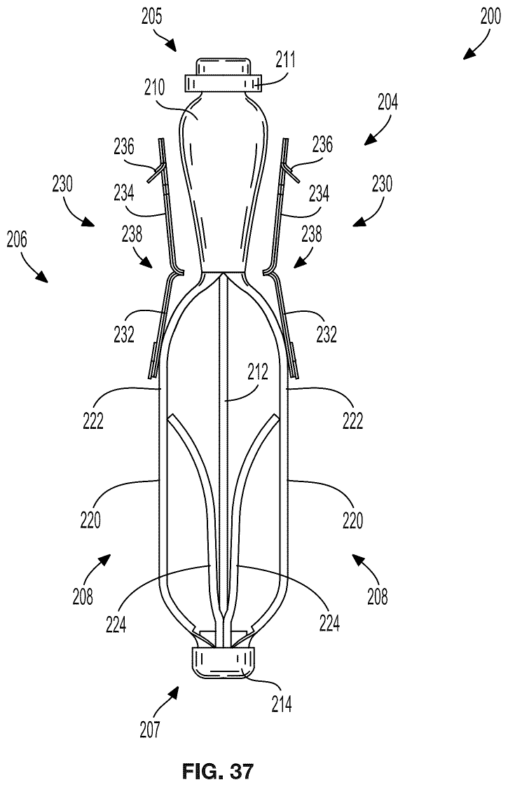

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and movable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

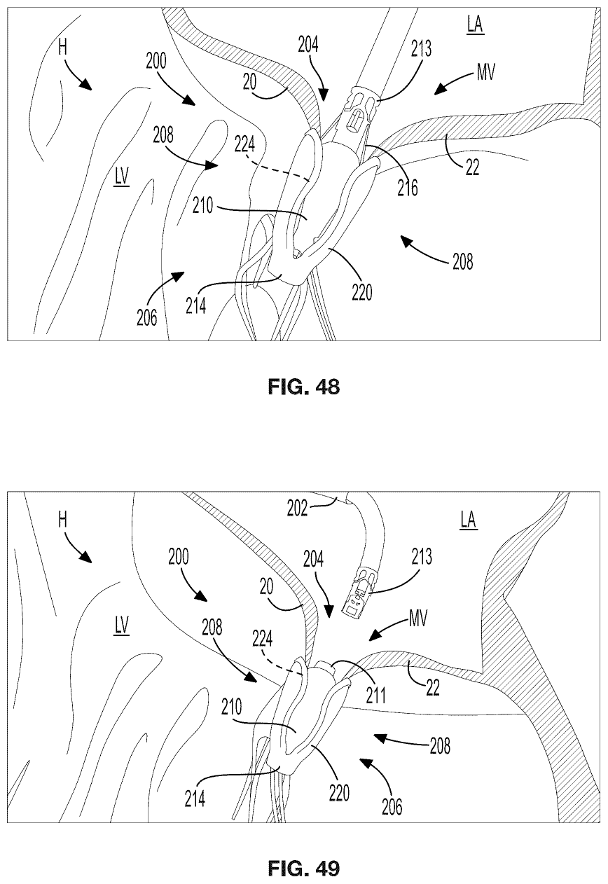

Referring now to FIGS. 38-49, an example device 200 is shown being delivered and implanted within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter 241 and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

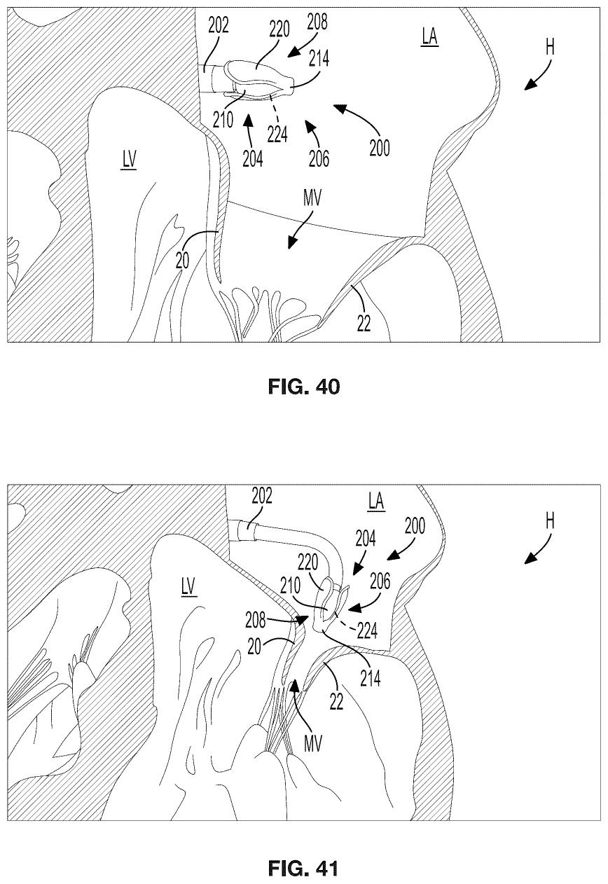

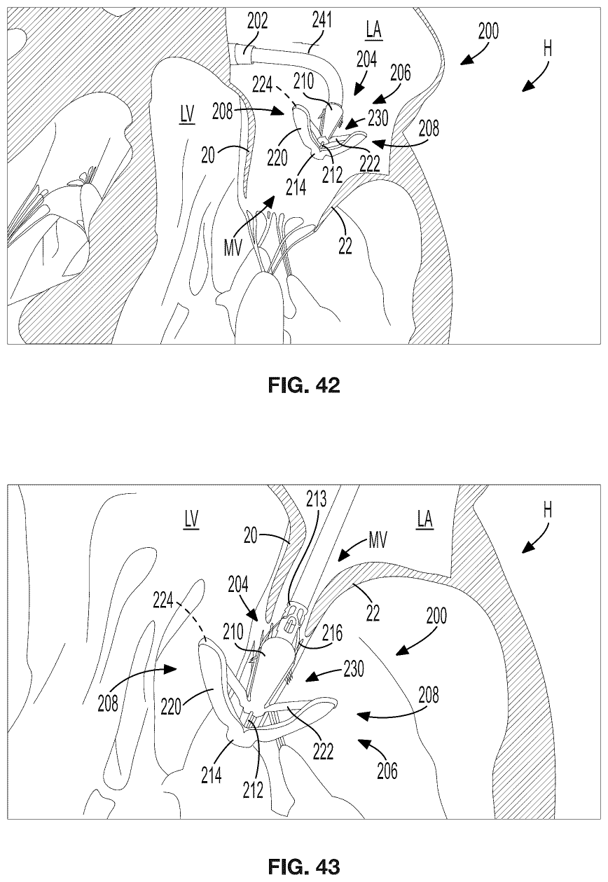

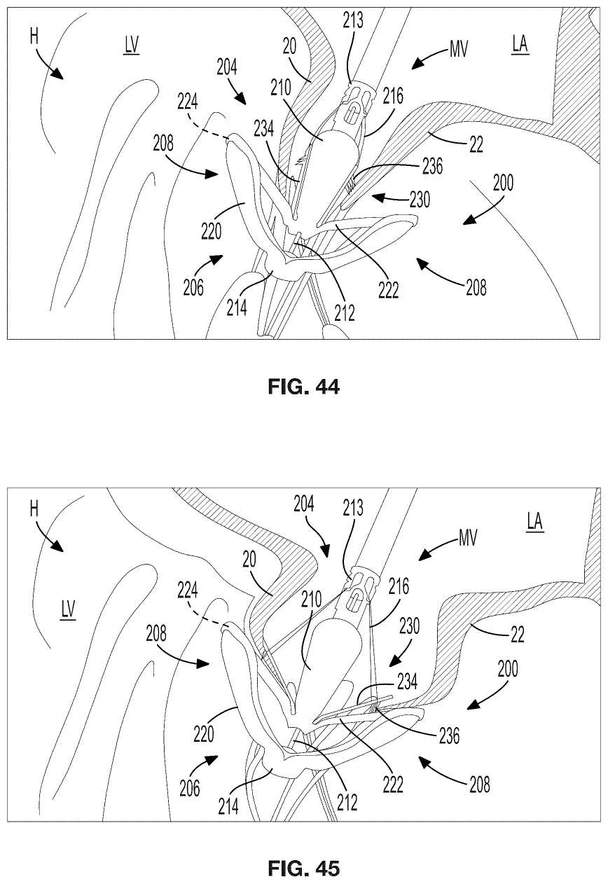

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both tissue engagement portions/clasps 230 closed, though the optional barbs 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal component or collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy—produced in a wire, sheet, tubing, or laser sintered powder—and are biased to hold the outer paddles 220 closed around the inner paddles 222, coaptation element 210, and/or the clasps 230 pinched around native leaflets 20, 22.

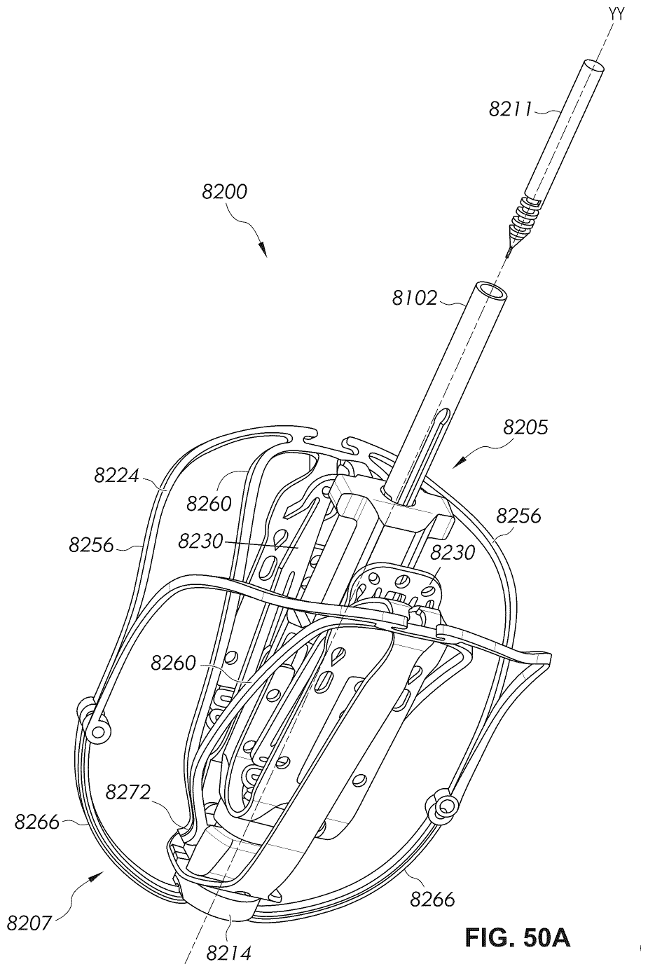

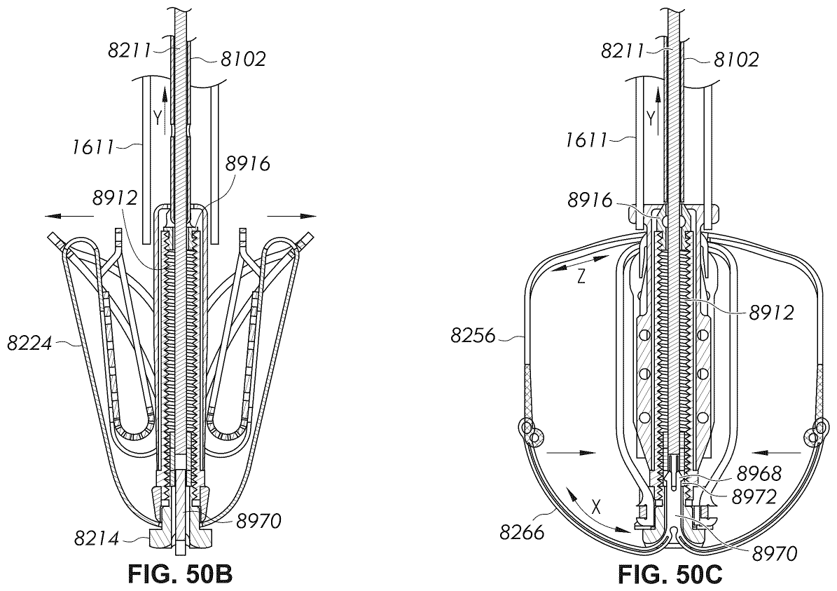

FIGS. 50A, 50B, and 50C illustrate an example system and/or apparatus to which the concepts of the present application can be applied. The system includes an implant catheter assembly 1611 and a device 8200 (e.g., a valve repair device, a valve treatment device, an implantable device, etc.). The device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 50A, the paddle frames 8224 can be symmetric along longitudinal axis YY. However, in some implementations, the paddle frames 8224 are not symmetric about the axis YY. Moreover, referring to FIG. 50A, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 50C).

In some implementations, between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

In some implementations, the inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 50A.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 50C) and portions of the connector 8266 into the actuation cap 8214. The actuation element 8102 is configured to move the inner frame portions 8260 to open and close the paddles in accordance with some implementations disclosed herein.

As shown in FIGS. 50B and 50C, in some implementations, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position.

In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls, or other portions.

In some implementations, when the coupler 8972 is attached to the width adjustment element 8211, the coupler is released from the receiver 8912. In some implementations, when the coupler 8972 is detached from the width adjustment element 8211, the coupler is secured to the receiver.

The inner end 8968 of the connector can be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

In some implementations, the width adjustment element 8211 allows a user to expand or contract the outer frame portions 8256 of the device 8200. In the example illustrated in FIGS. 50B and 50C, the width adjustment element 8211 includes an externally threaded end that is threaded into the coupler 8972. In some implementations, the width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as the other implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 50B. That is, movement of the distal cap 8214 in the direction Y closes the device and movement of the distal cap in the direction opposite to direction Y opens the device.

Also illustrated in FIGS. 50B and 50C, in some implementations, the width adjustment element 8211 extends through the actuation element 8102, the driver, head 8916, and the receiver 8912 to engage the coupler 8972 attached to the inner end 8968. In some implementations, the movement of the outer frame portions 8256 to the narrowed position can allow the device or implant 8200 to maneuver more easily into position for implantation in the heart by reducing the contact and/or friction between the native structures of the heart—e.g., chordae—and the device 8200. In some implementations, the movement of the outer frame portions 8256 to the expanded position provides the anchor portion of the device or implant 8200 with a larger surface area to engage and capture leaflet(s) of a native heart valve.

The device 8200 (e.g., the anchors 8830, 8834 or another portion of the device) can include tissue engagement portions or clasps 8230 that can be the same as or similar to the clasps 130, 230, 330, 40856, 5030a, 5030b, 5030c, or other tissue engagement portions or clasps herein.

The bioimpedance-based feedback disclosed herein can be used to provide feedback related to the clasping of tissue in tissue engagement portions or clasps of the one or more of the distal anchors 8830 and/or the proximal anchors 8834.

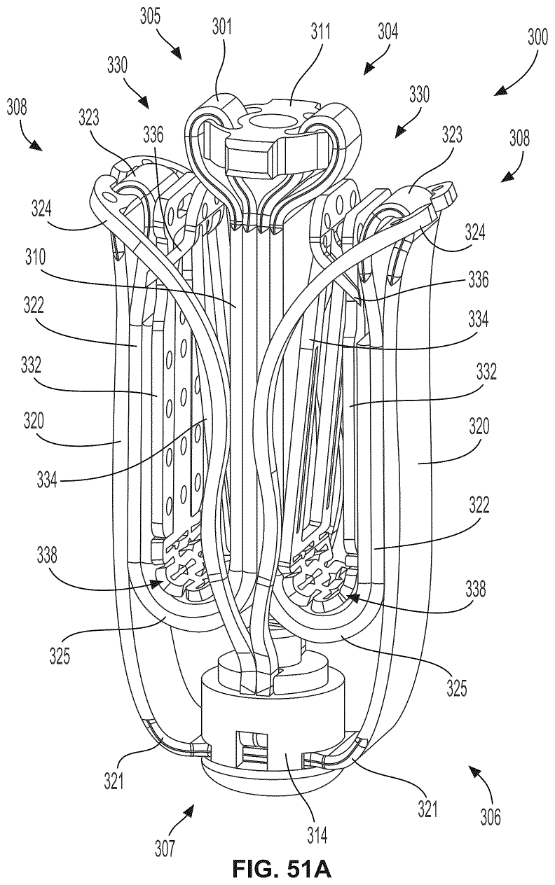

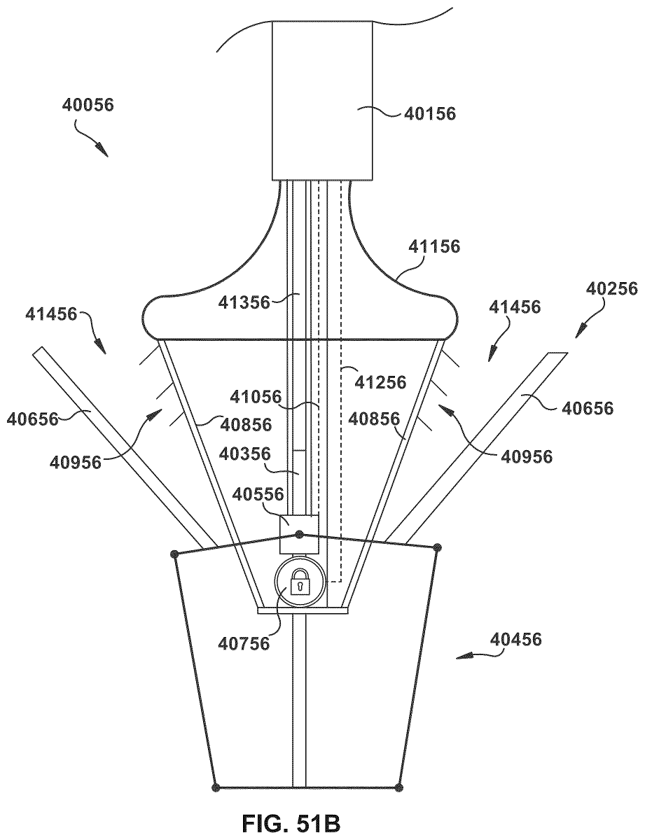

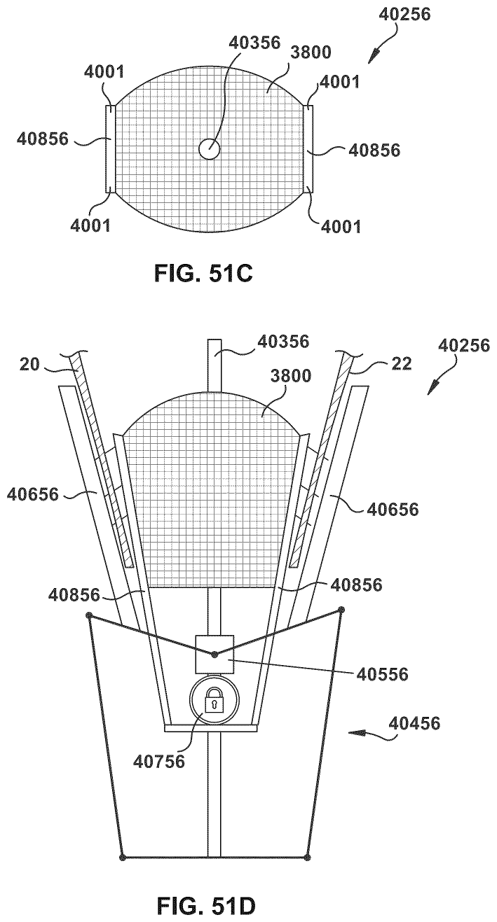

FIG. 51A illustrates an example of a device or implant 300 (e.g., a treatment device, a repair device, an implantable device, etc.). The devices herein, including device 100 that is schematically illustrated in FIGS. 8-15, can be the same as or similar to device 300 (and/or the same as or similar to any other example devices disclosed herein, described in incorporated references, or any device otherwise compatible with the concepts herein).

The device 300 can include any other features for a device or implant discussed in the present application, and the device 300 can be positioned to engage valve tissue 20, 22 as part of any suitable valve repair system (e.g., any valve repair system disclosed in the present application).

In some implementations, the device 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve.

In some implementations, the anchor portion 306 includes a plurality of anchors 308. In some implementations, each anchor 308 can include one or more paddles, e.g., outer paddles 320, inner paddles 322, paddle extension members (e.g., leaf spring, shaped wire, etc.), paddle frames 324, etc. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

In some implementations, the anchors 308 can comprise first portions or outer paddles 320 and second portions or inner paddles 322 separated by connection portions 323. In some implementations, the connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations that include an optional coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip of fabric 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent to the longitudinal axis of the spacer or coaptation element 310. From the straight configuration, the anchors 308 can be moved to a fully folded configuration (as shown in FIG. 51A), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. In some implementations, when included, the fixed arms 332 can be attached to the inner paddles 322, with the joint portion 338 disposed proximate the coaptation element 310. In some implementations, the joint portion 338 is spring-loaded so that the fixed and moveable arms 332, 334 are biased toward each other when the clasp 330 is in a closed condition.

In some implementations, the fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336.

In some implementations, the clasps 330 are opened by applying tension to actuation lines attached to the moveable arms 334, thereby causing the moveable arms 334 to articulate, pivot, and/or flex on the joint portions 338.

In short, the device or implant 300 can be similar in configuration and operation to the device or implant 200 described above, but the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 51A shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. In the illustrated example of FIG. 51A, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (e.g., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

FIG. 51B illustrates another example system and/or apparatus to which the concepts of the present application can be applied. The system 40056 includes a delivery device 40156 and a device 40256 (e.g., a valve repair device, a valve treatment device, an implantable device, etc.)

In some implementations, the valve repair device 40256 includes a base assembly 40456, a pair of paddles 40656 (e.g., clasp, clip, arm, etc.), and a pair of gripping members 40856 (e.g., clasp, clip, arm, etc.). In some implementations, the paddles 40656 can be integrally formed with the base assembly. For example, the paddles 40656 can be formed as extensions of links of the base assembly. In the illustrated example, the base assembly 40456 of the valve repair device 40256 has a shaft 40356, a coupler 40556 configured to move along the shaft, and a lock 40756 configured to lock the coupler in a stationary position on the shaft. In some implementations, a gripping member 40856 can be considered a first arm and a paddle 40656 can be considered a second arm of a clasp, clip, tissue engagement portion, etc.

In some implementations, the coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include an optionally barbed portion 40956 (or otherwise friction-enhancing portion with or without barbs) for attaching the gripping members to valve tissue when the valve repair device 40256 is attached to the valve tissue.

In some implementations, when the paddles 40656 are in the closed position, the paddles engage the gripping members 40856, such that, when valve tissue is attached to the barbed portion 40956 (while described as a “barbed portion” here, other friction enhancing elements instead of or in addition to barbs can be used) of the gripping members, the paddles secure the valve repair device 40256 to the valve tissue.

In some implementations, the gripping members 40856 are configured to engage the paddles 40656 such that the barbed portion 40956 engages the valve tissue and the paddles 40656 to secure the valve repair device 40256 to the valve tissue. For example, in certain situations, it can be advantageous to have the paddles 40656 maintain an open position and have the gripping members 40856 move outward toward the paddles 40656 to engage valve tissue and the paddles 40656.

Although the example shown in FIG. 51B illustrates a pair of paddles 40656 and a pair of gripping members 40856, it should be understood that the valve repair device 40256 can include any suitable number of paddles and gripping members.

In some implementations, the system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The system 40056 can also include a paddle control mechanism 41056 (e.g., relatively movable tube(s), shaft(s), etc.), a gripper control mechanism 41156 (e.g., wire(s), line(s), suture(s), etc.), and a lock control mechanism 41256 (e.g., relatively movable tube(s), shaft(s), wire(s), line(s), suture(s), etc.).

In some implementations, the paddle control mechanism 41056 is mechanically attached to the coupler 40556 to move the coupler along the shaft, which causes the paddles 40656 to move between the open and closed positions. The paddle control mechanism 41056 can take any suitable form, such as, for example, a shaft or rod. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture or wire, a rod, a catheter, etc.

The lock control mechanism 41256 is configured to lock and unlock the lock. The lock 40756 locks the coupler 40556 in a stationary position with respect to the shaft 40356 and can take a wide variety of different forms and the type of lock control mechanism 41256 can be dictated by the type of lock used. In examples in which the lock 40756 includes a pivotable plate, the lock control mechanism 41256 is configured to engage the pivotable plate to move the plate between the tilted and substantially non-tilted positions. In some implementations, the lock control mechanism 41256 can be, for example, a rod, a suture, a wire, or any other member that is capable of moving a pivotable plate of the lock 40756 between a tilted and substantially non-tilted position.

The valve repair device 40256 is movable from an open position to a closed position. In the illustrated example, the base assembly 40456 includes links that are moved by the coupler 40556. The coupler 40556 is movably attached to the shaft 40356. In order to move the valve repair device from the open position to the closed position, the coupler 40556 is moved along the shaft 40356, which moves the links.

In some implementations, the gripper control mechanism 41156 moves the gripping members 40856 to provide a wider or a narrower gap at the opening 41456 between the gripping members and the paddles 40656. In the illustrated example, the gripper control mechanism 41156 includes a line, such as a suture, a wire, etc. that is connected to an opening in an end of the gripper members 40856. When the line(s) is pulled, the gripping members 40856 move inward, which causes the opening 41456 between the gripping members and the paddles 40656 to become wider.

In order to move the valve repair device 40256 from the open position to the closed position, the lock 40756 is moved to an unlocked condition by the lock control mechanism 41256. Once the lock 40756 is in the unlocked condition, the coupler 40556 can be moved along the shaft 40356 by the paddle control mechanism 41056.

After the paddles 40656 are moved to the closed position, the lock 40756 is moved to the locked condition by the lock control mechanism 41256 to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356. In addition, the valve repair device 40256 is disengaged from the paddle control mechanism 41056, the gripper control mechanism 41156, and the lock control mechanism 41256.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Tissue engagement portions, such as clasps or leaflet gripping devices, disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

FIGS. 51C and 51D illustrate an example implementation of a valve repair device 40256 that includes a coaptation element 3800. The valve repair device 40256 can have the same or a similar configuration as the valve repair device illustrated by FIG. 51B with the addition of the coaptation element. The coaptation element 3800 can take a wide variety of different forms.

In some implementations, the coaptation element 3800 can be compressible and/or expandable. For example, the coaptation element can be compressed to fit inside one or more catheters of a delivery system, can expand when moved out of the one or more catheters, and/or can be compressed by the paddles 40656 to adjust the size of the coaptation element. In the example illustrated by FIGS. 51C and 51D, the size of the coaptation element 3800 can be reduced by squeezing the coaptation element with the paddles 40656 and can be increased by moving the paddles 40656 away from one another. The coaptation element 3800 can extend past outer edges 4001 of the gripping members or clasps 40856 as illustrated for providing additional surface area for closing the gap of a mitral valve.

The coaptation element 3800 can be coupled to the valve repair device 40256 in a variety of different ways. For example, the coaptation element 3800 can be fixed to the shaft 40356, can be slidably disposed around the shaft, can be connected to the coupler 40556, can be connected to the lock 40756, and/or can be connected to a central portion of the clasps or gripping members 40856. In some implementations, the coupler 40556 can take the form of the coaptation element 3800. That is, a single element can be used as the coupler 40556 that causes the paddles 40656 to move between the open and closed positions and the coaptation element 3800 that closes the gap between the leaflets 20, 22 when the valve repair device 40256 is attached to the leaflets.

The coaptation element 3800 can be disposed around one or more of the shafts or other control elements of the valve repair system 40056. For example, the coaptation element 3800 can be disposed around the shaft 40356, the shaft 41356, the paddle control mechanism 41056, and/or the lock control mechanism 41256.

The valve repair device 40256 can include any other features for a valve repair device discussed in the present application, and the valve repair device 40256 can be positioned to engage valve tissue as part of any suitable valve repair system (e.g., any valve repair system disclosed in the present application). Additional features of the device 40256, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

FIG. 51E illustrates another example of one of the many valve repair systems for repairing a native valve of a patient that the concepts of the present application can be applied to. The valve repair system includes a device 8810 that includes a frame 8820, anchors 8830, 8834, a band 8840, an annular flap or sail 8850 and a valve body 8860. The device 8810 Scan include a proximal end 8812 and a distal end 8814 with openings defined at both ends 8812, 8814 such that fluid can flow therethrough. In some implementations, the proximal end 8812 can be placed in the left atrium while the distal end 8814 can be placed in the left ventricle such that device 8810 can function as a replacement for a mitral valve. The device 8810 can allow blood flow in a first direction from the proximal end 8812 to the distal end 8814 while preventing blood flow in a second direction from the distal end 8814 to the proximal end 8812.

The device or implant 8810 can include one or more distal anchors 8830. The distal anchors 8830 can be positioned along or proximate a distal end of the frame 8820 and can be connected to the frame 8820. The distal anchors 8830 can be designed such that when the frame 8820 is in an expanded configuration an end or tip 8832 of each distal anchor 8830 is positioned radially outward from the frame 8820 and extends generally in a proximal direction. In some implementations, the device 8810 can include one or more proximal anchors 8834. The proximal anchors 8834 can be positioned along or proximate a proximal end 8812 of the frame 8820 and can be connected to the frame 8820. The proximal anchors 8834 can be designed such that when the frame 8820 is in an expanded configuration an end or tip 8836 of each proximal anchor 8834 is positioned radially outward from the frame 8820 and extends generally in a distal direction. In some implementations, one or more anchors 8830, 8834 can include cushions 8838 covering one or more of such anchors.

In some implementations, the device 8810 can be disposed so that the mitral annulus is between the distal anchors 8830 and the proximal anchors 8834. In some implementations, the device 8810 can be positioned such that ends or tips 8832 of the distal anchors 8830 contact the annulus. In some implementations, the device 8810 can be positioned such that ends or tips 8832 of the distal anchors 8830 do not contact the annulus. In some implementations, the device 8810 can be positioned such that the distal anchors 8830 do not extend around the leaflet. In some implementations, the device 8810 can be positioned such that some distal anchors 8830 contact the annulus while other distal anchors 8830 do not. In some implementations, the device 8810 can be positioned so that the ends or tips 8832 of the distal anchors 8830 are on a ventricular side of the mitral annulus and the ends or tips 8836 of the proximal anchors 8834 are on an atrial side of the mitral annulus.

In some implementations, the distal anchors 8830 can be positioned such that the ends or tips 8832 of the distal anchors 8830 are on a ventricular side of the native leaflets beyond a location where chordae tendineae connect to free ends of the native leaflets. The distal anchors 8830 can extend between at least some of the chordae tendineae and, in some situations, can contact or engage a ventricular side of the annulus. It is also contemplated that in some implementations, the distal anchors 8830 may not contact the annulus, and the distal anchors 8830 may contact the native leaflet. In some situations, the distal anchors 8830 can contact tissue of the left ventricle beyond the annulus and/or a ventricular side of the leaflets. In some implementations, during delivery, the distal anchors 8830 (along with the frame 8820) can be moved toward the ventricular side of the annulus with the distal anchors 8830 extending between at least some of the chordae tendineae to provide tension on the chordae tendineae. Further examples of the device 8810 are provided in U.S. Pub. No. 2015/0328000 published Nov. 19, 2015, which is incorporated by reference herein in its entirety.

In certain implementations, the device 8810 does not include proximal anchors 8834. In such implementations, the distal anchors 8830 can be configured to clasp onto the native leaflet, the annulus, the chordae tendineae, or a combination of two or more of these. The bioimpedance-based feedback disclosed herein can be used to provide feedback related to the clasping of tissue in one or more of the distal anchors 8830 and/or the proximal anchors 8834.

Bioimpedance-Based Feedback with Devices

The following provides examples of devices that enable the use of bioimpedance or bioimpedance-based feedback in medical procedures. Although some of the description herein focuses on implementations in devices designed for leaflet capture for illustrative purposes, it should be understood that the bioimpedance-based feedback capabilities, properties, and functionalities can be applied to other devices used in a variety of medical procedures and with a variety of tissues. These include, for example and without limitation, annuloplasty devices, anchors for devices, implants, treatment devices, valves, stents, prosthetic valves, devices that anchor to muscle, devices that anchor to tissue, and the like. Some example devices which can be used with the disclosed bioimpedance-based feedback techniques (e.g., including the sensors, printed circuit boards, circuits, electrodes, measurement systems, etc.) are described herein with reference to FIGS. 8-15, 22-37, and 50A-51E. Further, the bioimpedance-based feedback capabilities, properties, and functionalities can be applied to other systems, devices, components, etc. that are not implanted, e.g., delivery systems, delivery devices, catheters, anchor drivers, pushers (e.g., push rods, etc.), leaflet repair tools that capture a leaflet for treatment and later release the leaflet, chordae repair/replacement devices, leaflet prolapse repair devices, other treatment and/or repair devices, etc.

While many examples herein describe a clasp for illustrative purposes, the same or similar concepts, configurations, measurements, principles, etc. (e.g., similar electrodes, etc.), such as those described with respect to “clasps”, can be used on other implementations, anchors, anchor portions, clips, clamps, gripping members, paddles, configurations, etc. even if not a traditional “clasp.” In some implementations, the tissue engagement portions, anchors, clasps, etc. can include arms that are not directly hinged to each other. In some implementations, the tissue engagement portions, clasps, etc. herein may not include a fixed arm. In some implementations, the gripping members may be pivotably connected to and/or formed with a base assembly.

In some implementations, the same or similar concepts, configurations, measurements, principles, etc. (e.g., similar electrodes, etc.), such as those described with respect to “clasps”, can be used in conjunction with the distal anchors 8830 or arms of the device 8810 of FIG. 51E.

In some implementations, the same or similar concepts, configurations, measurements, principles, etc. (e.g., similar electrodes, etc.), such as those described with respect to “clasps”, can be used on an implantable device or non-implantable device that includes a tissue engagement portion or tissue capture portion that is formed by a first surface and a second surface that move relative to each other, whether or not associated with a first arm and a second arm and/or whether or not the surfaces are directly connected or hinged to each other.

In some implementations, the same or similar concepts, configurations, measurements, principles, etc. (e.g., similar electrodes, etc.), such as those described with respect to “clasps”, can be used on an implantable device or non-implantable device that includes a tissue engagement portion or tissue capture portion that is formed by a first surface (e.g., of a gripping member, of an arm, of a clasp arm, of a first arm, etc.) and a second surface (e.g., of a paddle, of an arm, of clasp arm, of a second arm, of a coaptation element, etc.) wherein at least one of the first surface and the second surface can move relative to the other surface, whether or not the surfaces are directly connected or hinged to each other.

In typical transcatheter edge-to-edge repair (TEER) procedures and other such procedures that include leaflet grasping, echo-based imaging is predominantly used. Such imaging techniques can be helpful. However, as described herein, other techniques used with or without imaging can be helpful and potentially improve confidence and results. In some instances, the disclosed bioimpedance-based feedback techniques herein can be used to augment imaging techniques. For example, even on the mitral side where echo imaging is normally good, procedures may involve deploying more than one implant. In such instances, the first implant may cause shadowing when deploying a second implant, making accurate measurements of leaflet insertion difficult and making it more difficult to determine leaflet capture. Accordingly, disclosed herein are systems, methods, and devices that use bioimpedance-based feedback to provide feedback related to tissue engagement, tissue capture, and/or anchor deployment for devices, such as the devices disclosed herein. The bioimpedance-based feedback can be used to generate indicators to help users make decisions regarding leaflet capture, the feedback being independent of echo imaging.

Furthermore, it may be beneficial to generate indicators in addition to those related to leaflet insertion or leaflet capture. For example, it may be beneficial for a user to understand coaptation, tension, para-implant leak, the strength of leaflet tissue, the holding force of the clasp on the leaflet, and the like. Accordingly, algorithms described herein can be used to provide indicators that provide useful information for users to determine not only leaflet capture but also coaptation, tension, regurgitation, leaflet tissue strength, and the like. Advantageously, these indicators can be used to achieve desirable outcomes in medical procedures.

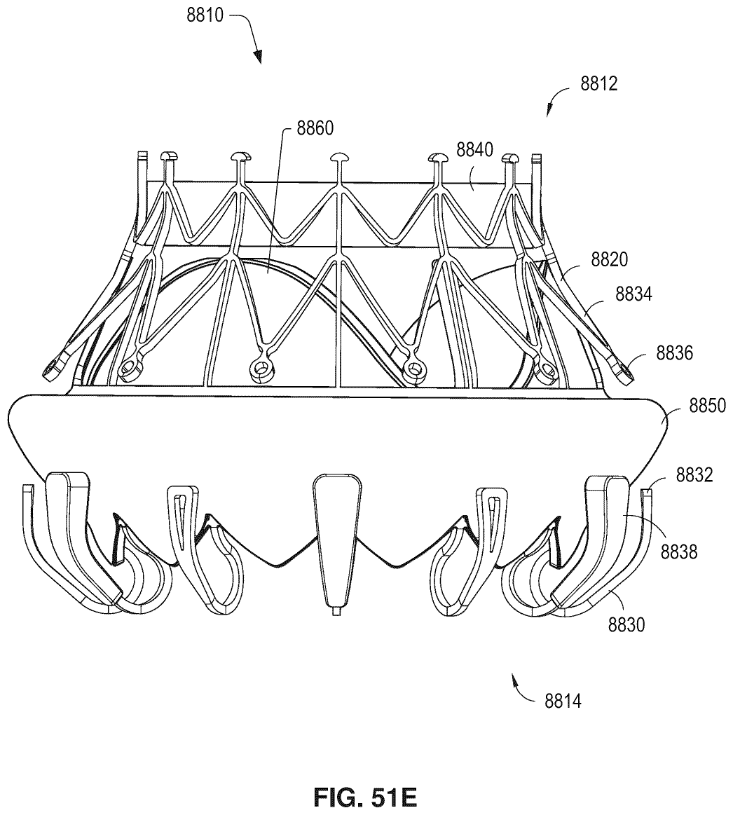

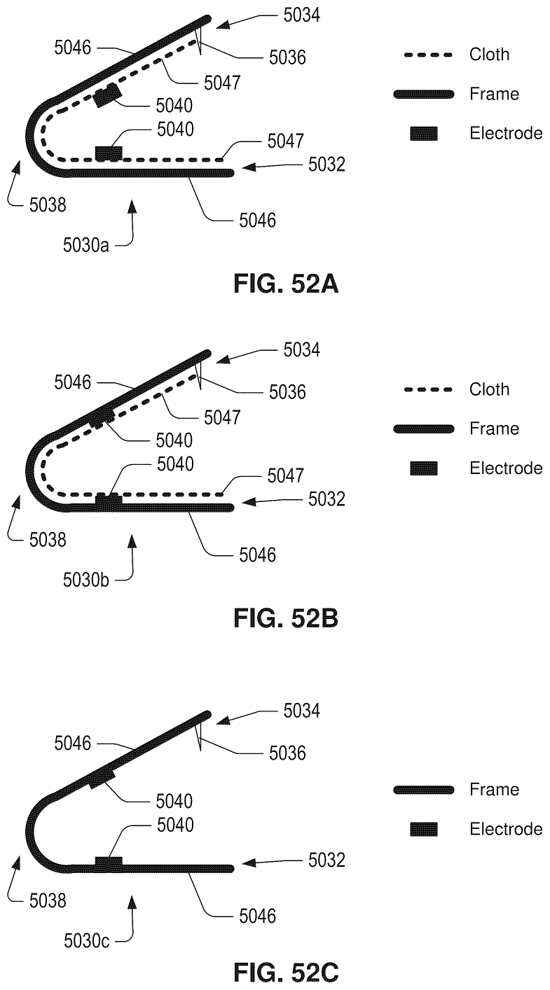

FIGS. 52A, 52B, 52C illustrate example anchor portions, anchors, tissue engagement portions, or clasps 5030a, 5030b, 5030c having at least one electrode 5040, e.g., two or more electrodes 5040. The anchor portions, anchors, tissue engagement portions, or clasps 5030a, 5030b, 5030c are useable with any of the systems devices herein mutatis mutandis, e.g., these are usable with the devices in FIGS. 8-51D, and with other implantable devices or non-implantable treatment devices that capture tissue.

FIG. 52A illustrates an anchor, anchor portion, tissue engagement portion, clasp, etc. 5030a with electrodes 5040 over a cloth 5047 or cover (e.g., the electrodes 5040 are exposed) whereas FIG. 52B illustrates an anchor, anchor portion, tissue engagement portion, clasp, etc. 5030b with electrodes 5040 under the cloth 5047 or cover (e.g., the electrodes are covered by the cloth 5047). FIG. 52C illustrates an anchor, anchor portion, tissue engagement portion, clasp, etc. 5030c with electrodes 5040 secured to a first arm 5032 and/or to a second arm 5034 without a cloth or cover.

The anchor portions, anchors, tissue engagement portions, or clasps 5030a, 5030b, 5030c can be similar to the tissue engagement portions or clasps 130, 230, 330, 40856, etc. described herein and share many of the same components (e.g., arms 5032, 5034, means for securing 5036, and joint portion 5038), properties, and functionality. The anchors, anchor portions, clasps 5030a, 5030b, 5030c can be used in place of the anchors/clasps 130, 230 or features (e.g., electrodes, etc.) of the anchors, anchor portions, clasps 5030a, 5030b, 5030c can be incorporated into the anchors/clasps 130, 230. The anchor portions, anchors, clasps 5030a, 5030b, 5030c can be implemented as part of the devices described herein, such as the devices 100, 200.

In some implementations, the anchor portions, anchors, tissue engagement portions, clasps 5030a, 5030b, 5030c can include a frame 5046, which can be conductive (e.g., made of Nitinol or other conductive material), and a cloth 5047, which can be insulative, to cover the frame 5046 (such as the covering 240). Although many of the implementations of anchor portions described herein include a cloth or cover, such as the cloth 5047 or the covering 240, it should be noted that the anchor portions can be implemented without a cloth or cover and in such implementations the bioimpedance technologies described herein can be used with little or no modifications to the disclosed anchor portions.

The electrodes 5040 can take a variety of different forms. For example, the electrodes 5040 can comprise one or more plates (e.g., covering a majority of a surface of the device, such as a majority of a surface of an arm), one or more rails (a thin rectangular strip along a length or across a width of an arm), one or more discs, one or more circles, etc. The electrodes 5040 can be incorporated into printed circuit boards (PCBs) attached to the tissue engagement portions or clasps 5030a, 5030b, 5030c, including flexible PCBs.

In some implementations, the electrodes 5040 can be coupled to a first surface of a device (e.g., a surface of first arm 5032 of a clasp 5030a, 5030b, 5030c), to a second surface of the device (e.g., a surface of a second arm 5034 of the clasp 5030a, 5030b, 5030c), to both the first surface and the second surface, and/or to one or more other portions of the device.

In some implementations, the electrodes 5040 can be coupled to a fixed arm 5032 of the clasp 5030a, 5030b, 5030c, to a moveable arm 5034 of the clasp 5030a, 5030b, 5030c, to both the fixed and moveable arms 5032, 5034, and/or to one or more other portions of the device.

In some implementations, the electrodes 5040 can be releasably coupled to a first surface and/or a first arm 5032 (e.g., a fixed arm). In some implementations, the electrodes 5040 can additionally or alternatively be releasably coupled to a second surface and/or a second arm 5034 (e.g., a moveable arm). In such implementations, the electrodes 5040 (e.g., PCBs, leads, etc. which incorporate the electrodes 5040) can be removed after implantation of the device to which the electrodes 5040 are coupled, as described in greater detail herein.

In some implementations, electrical leads can be coupled to the electrodes 5040. Further, in some implementations, the electrical leads can be removed after implantation of the device to which the electrodes 5040 are coupled, as described in greater detail herein. Individual electrodes 5040 can be made of one or more separate conductor strips, rails, discs, plates, etc. The electrodes 5040 can be made of any suitable electrically conductive material.

In some implementations, one or more electrodes 5040 can be positioned at or near a minimum acceptable or targeted tissue insertion depth (e.g., leaflet insertion depth, etc.). In some implementations, one or more electrodes 5040 can be positioned at or near a maximum acceptable or targeted tissue insertion depth. In some implementations, one or more electrodes 5040 can be positioned at or near a minimum acceptable or targeted tissue insertion depth (e.g., leaflet insertion depth, etc.) and can also be positioned at or near a maximum acceptable or targeted tissue insertion depth.

In some implementations, an alternating current is applied across the electrodes 5040 and one or more impedance measurements are taken and/or derived. For example, electrical leads can be electrically coupled to the electrodes 5040, as described herein, and current or voltage can be applied to the electrodes 5040 using the electrical leads. Similarly, electrical signals associated with the electrodes 5040 can be measured using the electrical leads to determine impedance characteristics and/or changes to impedance characteristics. The applied voltage amplitude and/or alternating current frequency can be varied. Different materials can have different impedance characteristics for different applied voltages or currents. As such, applying varying voltage amplitudes can allow for enhanced differentiation between different biological materials disposed in the anchor, tissue engagement portion, clasp, etc.

In some implementations, the voltage is applied, and one or more impedance characteristics are measured and/or determined. This can be done, for instance, while the anchor or clasp is closed. In some implementations, the voltage is applied, and one or more impedance characteristics are measured and/or determined while the anchor or clasp is open, partially open, or not fully closed. The measured impedance characteristics can then be used to determine the tissue state relative to the anchor, tissue engagement portion, clasp, etc. For example, the tissue state can be fully inserted, minimal viable insertion, too little insertion, no insertion, wrong tissue type inserted, etc. In some implementations, the configuration of the electrodes can enable the determination of the tissue state before closing the anchor, tissue engagement portion, clasp, etc. This can advantageously avoid extraneous punctures of the leaflets by barbs during clasp closure, as described herein. Thus, taking the impedance measurements (e.g., measurements of electrical signals indicative of impedance and/or from which impedance can be calculated) to determine the tissue state while a tissue engagement portion or clasp is open, partially open, or not fully closed can have the benefit of being able to confirm that tissue is properly positioned in the clasp and/or to confirm that another unwanted tissue (such as chordae tendinea) is not positioned in the anchor before the anchor is closed. Taking the impedance measurements (e.g., measurements of electrical signals indicative of impedance and/or from which impedance can be calculated) to determine the tissue state while the anchor, tissue engagement portion, clasp, etc. is open, partially open, or not fully closed can prevent or inhibit the optional barbs from piercing or penetrating the tissue (e.g., leaflet, etc.) until it is confirmed that the tissue is properly positioned in the anchor, tissue engagement portion, clasp, etc. Taking the impedance measurements to determine the tissue state while the anchor, tissue engagement portion, clasp, etc. is open, partially open, or not fully closed can help the user avoid capturing non-targeted tissue (e.g., chordae tendinea, etc.) in the anchor, tissue engagement portion, clasp, etc. (e.g., avoid closing the anchor, tissue engagement portion, clasp, etc. while non-targeted tissue is inside the anchor, tissue engagement portion, clasp, etc.)

In some implementations, the one or more impedance characteristics that are measured and/or determined can be used to determine whether targeted tissue (e.g., a leaflet) is in the anchor, tissue engagement portion, clasp, etc. while the anchor, tissue engagement portion, clasp, etc. is open, partially open, or not fully closed. In such implementations, the measured or determined impedance characteristics can also be used to generate an indicator (e.g., for an operator) that the targeted tissue is in a capture region of the anchor, tissue engagement portion, clasp, etc. while it is open, partially open, or not fully closed.

In some implementations, the one or more impedance characteristics that are measured and/or determined can be used to determine whether targeted tissue has been over inserted in the anchor, tissue engagement portion, clasp, etc. and/or whether the targeted tissue is folded or bunched in the anchor, tissue engagement portion, clasp, etc. In such implementations, the measured or determined impedance characteristics can also be used to generate an indicator that the targeted tissue has been over inserted, is folded or bunched in the anchor, tissue engagement portion, clasp, etc.

In some implementations, the one or more impedance characteristics that are measured and/or determined can be used to determine whether non-targeted tissue has been captured in the anchor, tissue engagement portion, clasp, etc. (e.g., a chordae tendineae has been accidentally captured). In such implementations, the measured or determined impedance characteristics can also be used to generate an indicator that indicates that non-targeted tissue has been captured.

In some implementations, the one or more impedance characteristics that are measured and/or determined can be used to determine whether targeted tissue that has been captured is askew or angled in the anchor, tissue engagement portion, clasp, etc. (e.g., one side of a leaflet is deeper in a tissue engagement portion or clasp than the other side). In such implementations, the measured or determined impedance characteristics can also be used to generate an indicator that indicates that captured tissue is angled or askew relative to the anchor, tissue engagement portion, clasp, etc.

In some implementations, the electrodes 5040 can be included in a circuit along with the AC power supply, an electrical sensor, and the wiring or electrical leads. The sensor and the power supply (e.g., an AC power supply, etc.) can be a single device or separate devices. The wiring connects the electrodes 5040 to the power supply and the electrical sensor to measure, among other things, resistance, inductance, capacitance, voltage, current, and/or impedance, components of impedance, etc. As described herein, the electrical characteristics measured by the electrical sensor can be used to determine the location of the clasp and/or the anatomy that the clasp is in contact with, based on the resistance, inductance, capacitance, voltage, impedance and/or current readings taken by the sensor. The sensor can take a variety of different forms, including an impedance meter. In some implementations, the sensor is implemented in a PCB or other such component that is attached or coupled to the clasp 5030a, 5030b, 5030c. In such implementations, the electrodes 5040 and the sensor can be integrated into the same PCB or other such component.

By way of example, it has been surprisingly discovered that when electrical signals are measured during leaflet capture, the amplitude and shape of the electrical signals are distinct in instances where the electrodes 5040 contact the leaflet or other portion of the heart valve (e.g., chordae tendinea). The electrical signals can differentiate the type of tissue that is being contacted, and the extent of that contact with the electrode 5040 (e.g., if the electrode is at the edge or near the root of the leaflet). As such, by placing electrodes on a device (e.g., on one or more of the devices 100, 200, 300, 40256, or other device) the electrical signals can assist the user in determining if the leaflet or other tissue is captured or partially captured in the device, whether no tissue is captured by the device and/or whether the device is contacting chordae tendinea or other portion of the heart valve (e.g., non-targeted tissue) instead of the leaflet (e.g., targeted tissue).

The electrodes 5040 measure electrical signals to assist a user in determining if tissue (e.g., targeted tissue, a leaflet, etc.) is captured or partially captured by the device. Each of the electrodes 5040 provides a signal in material, such as blood, and/or in contact with material (e.g., tissue) at different locations. For example, in some implementations, the electrodes 5040 can provide a signal based on being positioned in blood in the atrium (and not in contact with tissue), based on being positioned in blood in the ventricle (and not in contact with tissue), based on being in contact with valve leaflet tissue and/or based on being in contact with chordae tissue.

In some implementations, three, four, five, or more electrodes are included. Any number of electrodes can be included for each clasp 5030a, 5030b, 5030c.

The electrical signals can take a wide variety of different forms and can be processed in a wide variety of different ways to determine the position of the device in the body (e.g., the position within a heart and/or the position of the leaflets relative to the device). In some implementations, bioimpedance signals are measured on the electrodes 5040 as described herein. The bioimpedance signal can be separated into a real portion and an imaginary portion, as is well known in electrical engineering calculations. Also, in some implementations, where the electrical power provided to the electrodes 5040 is provided using alternating current, the bioimpedance signal can also be represented with a magnitude and a phase. The bioimpedance signals can be analyzed to provide indications of the position of the electrodes 5040, and hence the tissue engagement portion or clasp 5030a, 5030b, 5030c relative to targeted tissue (e.g., leaflets) and/or relative to other tissue or non-targeted tissue (e.g., chordae tissue).

When measuring bioimpedance signals, different signal readings correspond to different relative positions of tissue (e.g., targeted tissue, leaflets, etc.) and the electrodes 5040. For example, in some implementations, if a leaflet contacts only one electrode, then a lower magnitude bioimpedance signal reading can result. However, when the leaflet sufficiently contacts two or more electrodes, then a higher magnitude bioimpedance signal reading can result, indicating that the device is correctly placed. This is due to the leaflet impeding the current more than the blood. Hence, the more a leaflet covers the electrodes, the higher the impedance becomes (e.g., a thicker leaflet will have a higher impedance). Thus, the configuration of the electrodes can be used to determine whether targeted tissue is partially captured or within a clasp while it is open or not fully closed, whether targeted tissue is askew or angled relative to a clasp, whether targeted tissue is over inserted or folded in the clasp, and/or whether non-targeted tissue has been captured in the clasp. Examples of bioimpedance signals resulting from different electrode and tissue configurations are described herein.

FIGS. 53A-53F illustrate anchors, tissue engagement portions, or clasps having different electrode configurations. FIGS. 53A and 53B illustrate an example device 5100 with anchors, tissue engagement portions, or clasps 5130 having electrodes 5140, 5145 positioned on an arm 5132 of the anchors, tissue engagement portions, or clasps 5130. The device 5100 can be the same as or similar to any of the devices (e.g., devices 100, 200, 300, 8200, 8810, 40256, or another device) described or incorporated herein. In addition, the tissue engagement portions or clasps 5130 can be the same as or similar to the clasps 130, 230, 330, 40856, 5030a, 5030b, 5030c (or other tissue engagement portions) described herein and share many of the same components (e.g., arms 5132, 5134, means for securing 5136, and joint portion 5138), properties, and functionality.

In some implementations of the anchors, tissue engagement portions, or clasps 5130, there are two strips of electrodes 5140, 5145 that fully or partially span a width of a first surface (e.g., a surface of the arm 5132). Thus, the electrodes 5140, 5145 provide bioimpedance signals corresponding to different amounts of tissue capture. A benefit of this type of configuration is that the clasp 5130 can be configured to indicate that there is tissue capture when the tissue (e.g., leaflet, etc.) contacts the electrodes 5140, 5145 even when the clasp 5130 is in a capture-ready configuration (e.g., the clasp 5130 is open or partially open).

FIGS. 53C and 53D illustrates an example device 5200 with anchors, tissue engagement portions, or clasps 5230 each having a first electrode 5240 positioned on a first surface (e.g., a surface of a first arm 5232) and a second electrode 5245 positioned on a second surface (e.g., a surface of a second arm 5234) of the device (e.g., of clasps 5230 of the device, of other portions of the device, etc.). The device 5200 can be the same as or similar to any of the devices (e.g., devices 100, 200, 300, 8200, 8810, 40256, or another device) described or incorporated herein. In addition, the tissue engagement portions or clasps 5230 can be the same as or similar to the clasps 130, 230, 330, 40856, 5030a, 5030b, 5030c (or other tissue engagement portions) described herein and share many of the same components (e.g., arms 5232, 5234, means for securing 5236, and joint portion 5238), properties, and functionality.

In some implementations of the tissue engagement portions or clasps 5230, there are two strips of electrodes 5240, 5245 that fully or partially span a width of a respective surface and/or width of a respective arm 5232, 5234. Thus, the electrodes 5240, 5245 provide bioimpedance signals corresponding to different sides of a leaflet or other tissue. A benefit of this type of configuration is that the tissue engagement portion or clasp 5230 can be configured to indicate that there is no tissue or leaflet capture when the tissue engagement portion or clasp 5230 is closed due at least in part to the electrodes 5240, 5245 being shorted, or in contact with each other, resulting in the impedance value being drastically reduced relative to a configuration in which the electrodes 5240, 5245 are apart and/or in contact with tissue.

FIGS. 53E and 53F illustrates an example device 5300 with tissue engagement portions or clasps 5330 each having a first electrode plate 5340 positioned on a first surface (e.g., a surface of first arm 5332) and a second electrode plate 5345 positioned on a second surface (e.g., a surface of a second arm 5334) of the device (e.g., of clasps 5230). The device 5200 can be the same as or similar to any of the devices (e.g., devices 100, 200, 300, 8200,8810, 40256, or another device) described or incorporated herein. In addition, the tissue engagement portions or clasps 5230 can be the same as or similar to the clasps 130, 230, 330, 40856, 5030a, 5030b, 5030c (or other tissue engagement portions) described herein and share many of the same components (e.g., arms 5332, 5334, means for securing 5336, and joint portion 5338), properties, and functionality.

In some implementations of the tissue engagement portions or clasps 5330, there are two electrode plates 5340, 5345 that fully or partially cover the area of a respective surface and/or of a respective arm 5332, 5334. Thus, the electrode plates 5340, 5345 provide bioimpedance signals corresponding to different capture depths of a leaflet or other tissue and can provide detailed information regarding relative capture depths of the leaflet or other tissue relative to other electrode configurations (e.g., the electrodes of the clasps 5130, 5230). An advantage of this configuration is that the tissue engagement portion or clasp 5330 is configured to indicate the tissue capture depth when the tissue or leaflet is between the electrode plates 5340, 5345, the tissue or leaflet acting as a dielectric. Similarly, another benefit of this configuration is that the clasp 5330 is configured to indicate that there is no tissue or leaflet capture when the clasp 5330 is closed due at least in part to the electrodes 5340, 5345 being shorted, or in contact with each other, resulting in the impedance value being drastically reduced relative to a configuration in which the electrodes 5340, 5345 are apart and/or in contact with tissue.

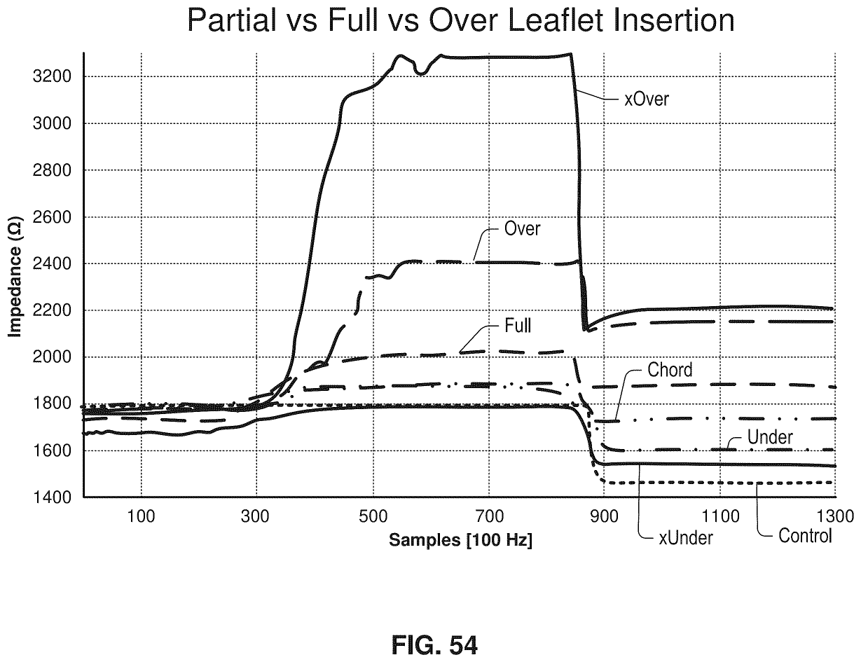

FIG. 54 illustrates example bioimpedance signals from a tissue engagement portion or clasp having two or more electrodes to provide bioimpedance-based feedback, such as the clasps 5030a, 5030b, 5030c, 5130, 5230, and/or 5330. The example bioimpedance signals are shown as a function of time which corresponds to an example process of moving a device into position next to leaflets and then grasping the leaflets, examples of which are described herein with respect to FIGS. 16-21 and 38-49. The different lines of the graph correspond to a fully captured leaflet (“Full”), an over captured leaflet (“Over”) which may result in the leaflet partially folding within the clasp, an extremely over captured leaflet (“xOver”) where the length of the leaflet captured in the clasp is about double or more than double the length of the clasp causing the leaflet to be bunched up on the clasp, an under captured leaflet (“Under”) (e.g., leaflet insertion is between about 4 mm and about 5.9 mm), an extremely under captured leaflet (“xUnder”) (e.g., leaflet insertion is between about 1 mm and about 3 mm), and where chordae tendineae are captured (“Chord”). In addition, a control bioimpedance signal (“Control”) is shown which corresponds to the bioimpedance signals when no leaflet is captured.

The initial baseline portion of the plot corresponds to the device being moved into position, prior to the leaflets entering the clasps. Examples of this configuration are shown in FIGS. 18 and 43. As the leaflets enter the open or partially open clasps, the bioimpedance signal rises sharply. Examples of this configuration are shown in FIGS. 19 and 44. As the clasps close on the leaflets, the bioimpedance signal drops to a steady-state signal that is different from the baseline signal resulting from the open clasps with no leaflets within the clasps. Examples of this configuration are shown in FIGS. 19, 20, and 45.

The amount of leaflet capture (or other tissue capture) can be determined based at least in part on the bioimpedance signals from the electrodes on the tissue engagement portion or clasps. Prior to capturing tissue (e.g., a leaflet), the bioimpedance signal is a steady state (or roughly steady state) signal, which can be referred to as an empty open clasp baseline. As the tissue or leaflet enters the open clasp or other tissue engagement portion, the bioimpedance signal increases (the contrast of which is shown in the control signal which does not increase because a leaflet does not enter the open clasp). As shown in FIG. 54, overly captured tissue (“Over” and “xOver”) results in a larger increase in the bioimpedance signal than fully captured tissue (“Full”) and under captured tissue (“Under” and “xUnder”). Similarly, under captured tissue (“Under” and “xUnder”) results in a smaller increase in the bioimpedance signal than fully captured tissue (“Full”). Thus, based on the increase or change in the bioimpedance signal, the amount of capture of the tissue can be determined. In addition, as the clasps close, the bioimpedance signal drops to a steady-state value (or roughly steady state), which can be referred to as a closed clasp baseline. This change in bioimpedance value also provides information regarding the status of the tissue capture and/or status of the tissue engagement portion or anchor. For example, if chordae tendineae are captured in the clasps, a different bioimpedance signal profile results relative to the clasps capturing a leaflet. The increase in the bioimpedance signal for the “Chord” and the “Under” situations are similar, but due to the different closed clasp or leaflet capture portion baseline bioimpedance signals, it can be determined that chordae tendineae were captured in the clasps or leaflet capture portion.

As described herein, the bioimpedance signals can be understood to reflect the amount of resistance to an electrical signal. The type of tissue and the amount of tissue between the electrodes affects the bioimpedance signal. If, for example, the clasps (or other tissue engagement portion) close with no tissue between the electrodes, it is similar to a shorted circuit with very little resistance between the electrodes. This is why the control signal has the lowest impedance in the graph after the clasps close. In the open position with no leaflets between the clasps, the blood between the electrodes provides a low-resistance electrical path. This is why each impedance signal in the graph has roughly the same open clasp baseline. As tissue enters the clasp, the amount of tissue in the clasp (reflecting whether the leaflet is fully, overly, or underly captured) affects the impedance, with more tissue typically increasing the amount of impedance to the electrical signal. With the clasps closed, again the amount of tissue in the clasp affects the impedance, with more tissue typically increasing the amount of impedance to the electrical signal. Thus, the bioimpedance signal profiles (which includes impedance signals from different portions of the implanting process) can be analyzed to determine the status of leaflet capture of a clasp. Accordingly, bioimpedance signals acquired, measured, or determined as described herein using electrodes on clasps can be used to determine whether targeted tissue is within a clasp even before the clasp closes, to determine whether tissue has been over inserted, to determine if non-targeted tissue is being or has been captured, and/or to determine of the targeted tissue is askew or angled relative to the clasp.

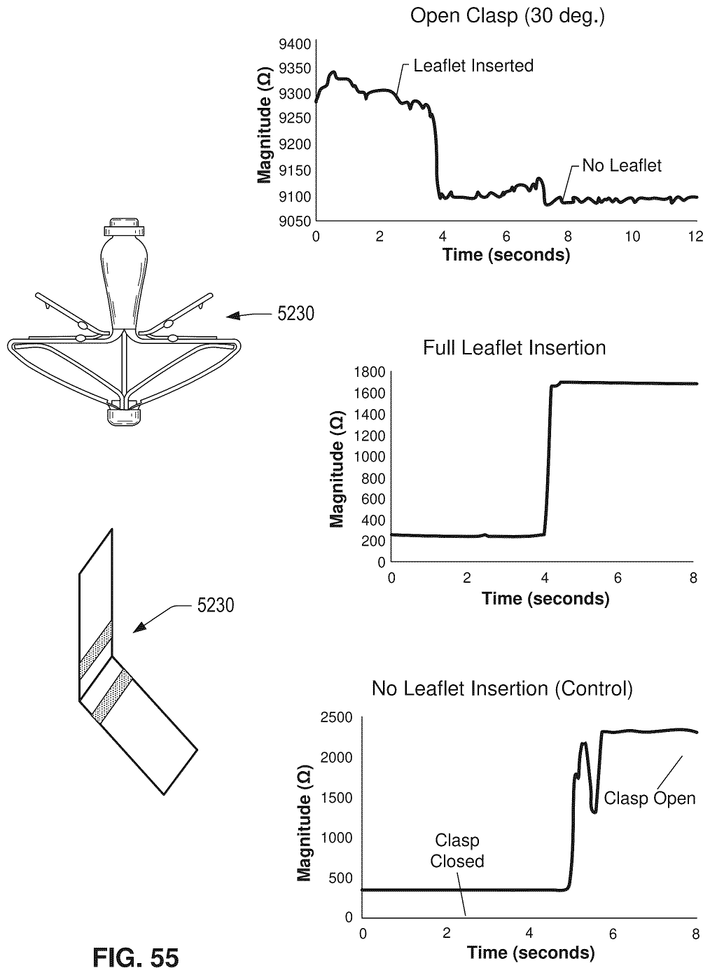

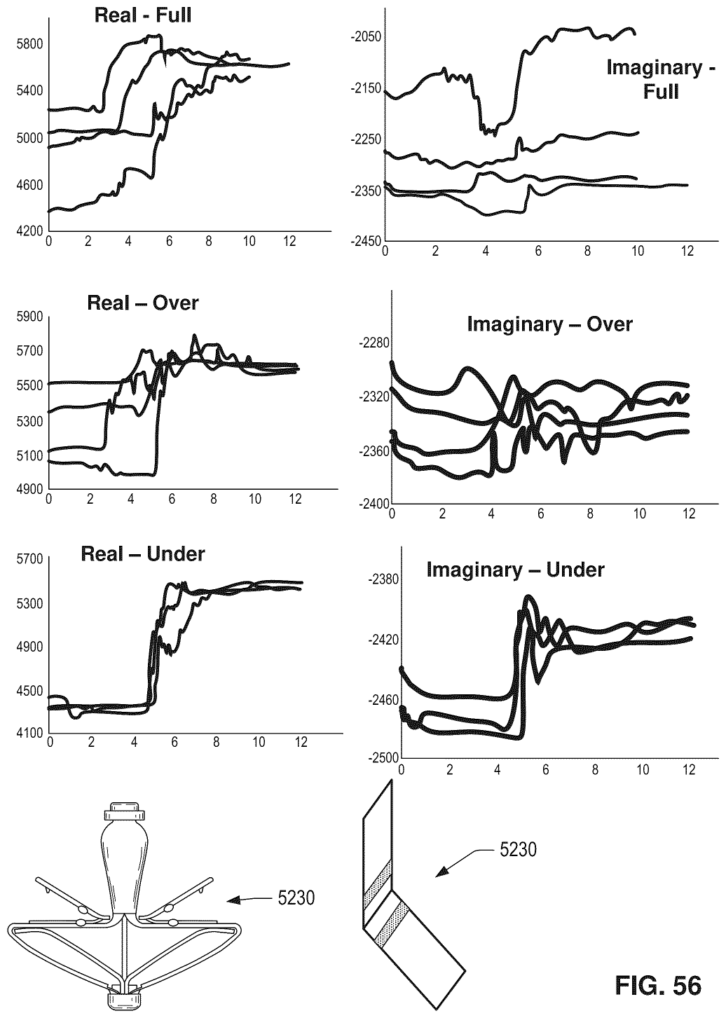

FIGS. 55 and 56 illustrate example bioimpedance signals from the tissue engagement portion or clasp 5230 of FIGS. 53C and 53D (which can be incorporated on any of the devices herein). The top graph of FIG. 55 illustrates the bioimpedance signals with the tissue engagement portion or clasp 5230 in the open position, the bioimpedance signal changing relative to the insertion depth of the leaflet (or other tissue) in the clasp 5230. The middle graph of FIG. 55 illustrates the increase in the bioimpedance as the leaflet is inserted fully into the clasp 5230. The bottom graph of FIG. 55 illustrates a control signal where no leaflet (or other tissue) is inserted in the clasp 5230 but the clasp is closed then opened. The top graphs of FIG. 56 illustrate the real (left graph) and imaginary (right graph) portions of the bioimpedance signal for a fully captured leaflet (or other tissue). The middle graphs of FIG. 56 illustrate the real (left graph) and imaginary (right graph) portions of the bioimpedance signal for an overly captured leaflet (or other tissue). The bottom graphs of FIG. 56 illustrate the real (left graph) and imaginary (right graph) portions of the bioimpedance signal for an under captured leaflet (or other tissue). The different lines in the graphs correspond to a variety of different measurements (e.g., using different leaflets) with a sample size of 4. This configuration of electrodes 5240, 5245 provides a relatively binary output for the status of the leaflet or tissue (e.g., captured or not captured). As shown, the example bioimpedance signals exhibit a significant difference between full leaflet capture, under leaflet capture, and over leaflet capture, making the situations relatively clear to classify in an analysis.

In some implementations, various algorithms can be implemented to analyze the bioimpedance signals of the tissue engagement portion or clasp 5230. For example, signal processing algorithms can be implemented. In such instances, some implementations can generate a binary output such as tissue contact or no tissue contact with the electrodes. In such instances, some implementations use a plurality of electrodes (e.g., arranged in an array) wherein the signals from each electrode or pairs of electrodes can be compared to one another to determine a tissue state. For example, if there are six electrodes evenly spaced on an inner paddle and a leaflet makes contact with the outer four electrodes, but the leaflet is folded at the middle pair, then each outer pair would have similar signals to one another but different from the middle pair. Certain implementations can then use a user interface to display the electrodes (or the signals measured at the electrodes) to show a user the tissue state (e.g., a folded leaflet), examples of which are described herein.

As another example of an algorithm, a threshold algorithm can be implemented that outputs an indication of an under captured leaflet or a fully captured leaflet after the clasps 5230 (or other tissue capture portion) have closed. This is similar to a mechanical indication of leaflet capture with an advantage that it provides a clearer user interface and is fast and simple to implement. As another example, a feature-based decision tree algorithm can be implemented that outputs an indication of an under, over, or fully captured leaflet after the clasps 5230 have closed. This algorithm can be configured to differentiate between thick and thin leaflets and helps to avoid over insertion of leaflets. This may reduce residual regurgitation and Single-Leaflet Device Attachment (SLDA) complications. As another example, a feature-based random forest algorithm can be implemented that outputs an indication of an under, over, or fully captured leaflet while the clasp 5230 is open and after the clasp 5230 has closed. This advantageously provides an indication of leaflet capture prior to closing the clasp 5230 which provides confirmation of leaflet capture prior to implant release. Similar principles apply to capture of other types of tissue.

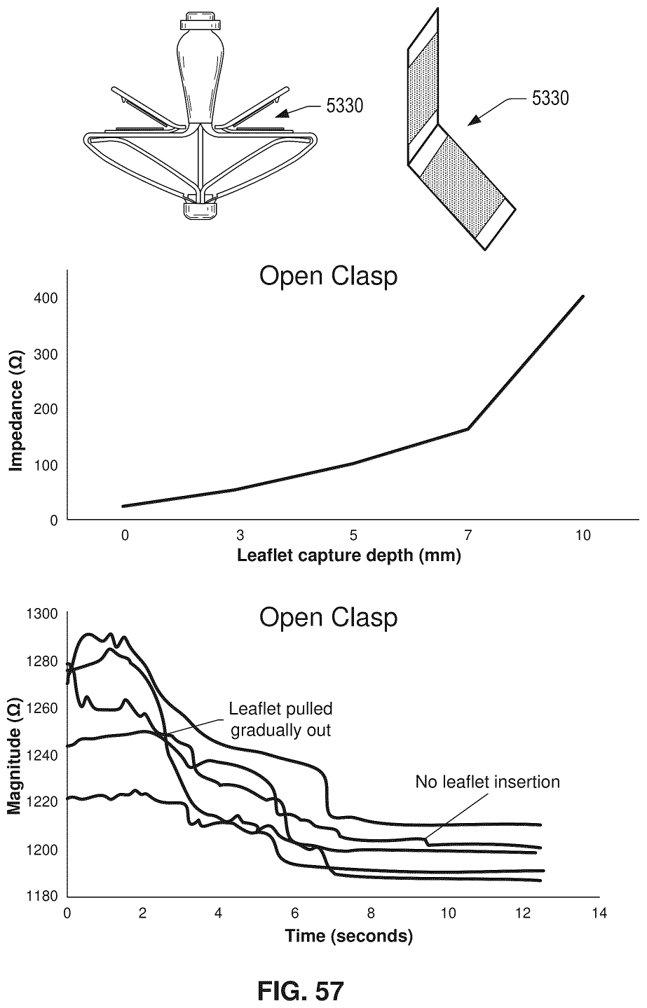

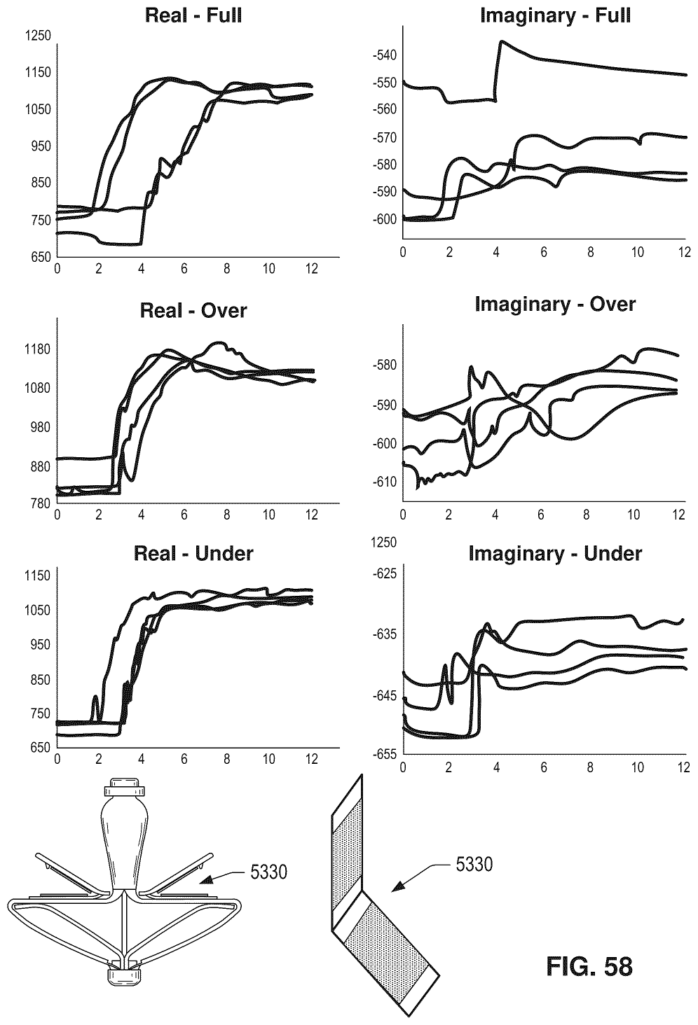

FIGS. 57 and 58 illustrate example bioimpedance signals from the tissue engagement portion or clasp 5330 of FIGS. 53E and 53F. The top graph in FIG. 57 illustrates the bioimpedance signal with the tissue engagement portion or clasp 5330 in the open position as a function of leaflet capture depth. This shows that the electrode plates 5340, 5345 provide signals that can be used to provide a relatively accurate determination of leaflet capture depth due at least in part to the configuration of the electrode plates 5340, 5345. The bottom graph of FIG. 57 illustrates the magnitude of the bioimpedance signal with the tissue engagement portion or clasp 5330 in the open position for various situations, such as pulling the leaflet gradually out of the clasp and where there is no leaflet insertion. The different lines in the bottom graph correspond to a variety of different measurements (e.g., using different leaflets) with a sample size of 5. The top graphs of FIG. 58 illustrate the real (left graph) and imaginary (right graph) portions of the bioimpedance signal with a fully captured leaflet. The middle graphs of FIG. 58 illustrate the real (left graph) and imaginary (right graph) portions of the bioimpedance signal with an over-captured leaflet. The bottom graphs of FIG. 58 illustrate the real (left graph) and imaginary (right graph) portions of the bioimpedance signal with an under-captured leaflet. The different lines in the graphs correspond to a variety of different measurements (e.g., using different leaflets) with a sample size of 4. Similar principles apply to capture of other types of tissue.

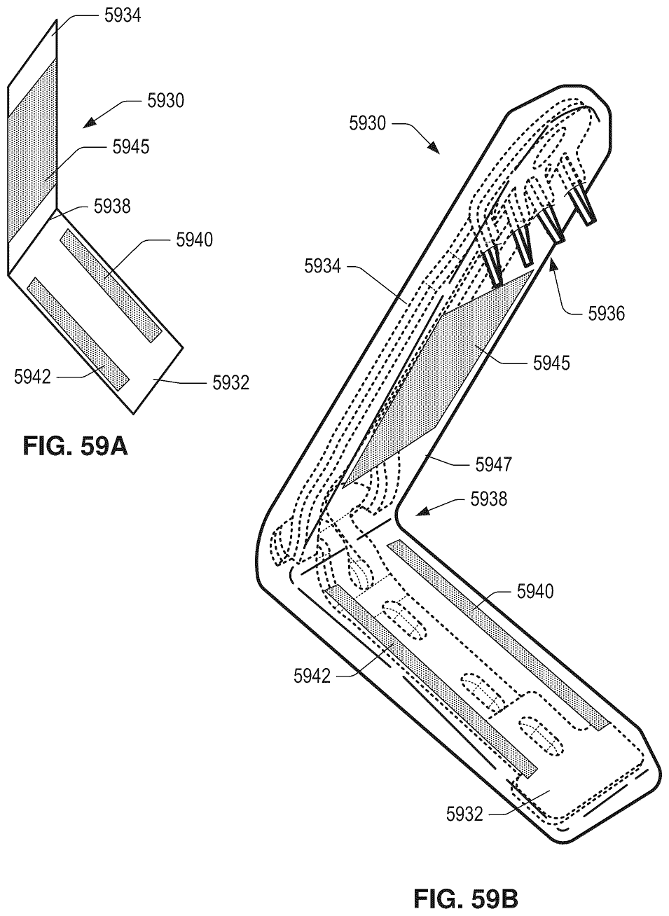

FIGS. 59A and 59B illustrate an example tissue engagement portion or clasp 5930 with a combination of an electrode plate 5945 and electrode strips 5940, 5942. The tissue engagement portions or clasp 5930 has electrode strips 5940, 5942 positioned on a first surface and/or a first arm 5932 and an electrode plate 5945 positioned on a second surface and/or a second arm 5334 of the clasp 5930 (e.g., the electrode strips 5940, 5942 and electrode plate 5945 over the cover 5947). The tissue engagement portion or clasp 5930 can be implemented on any of the devices described herein. In addition, the tissue engagement portion or clasp 5930 can be the same as or similar to the clasps 130, 230, 330, 40856, 5030a, 5030b, 5030c (or other tissue engagement portions) described herein and share many of the same components (e.g., arms 5932, 5934, means for securing 5936, and joint portion 5938), properties, and functionality.

In the illustrated example of the tissue engagement portion or clasp 5930, the electrode plate 5945 fully or partially covers the area of the surface or arm 5934. In addition, the electrode strips 5940, 5942 run parallel to the length of the surface or arm 5932 and cover a portion of the length of the surface or arm 5932, with a separation between the electrode strips 5940, 5942 along a width of the surface or arm 5932.

In some implementations, the combination of the electrode plate 5945 and electrode strips 5940, 5942 provide bioimpedance signals corresponding to different capture depths of a leaflet and can provide detailed information regarding relative capture depths of the leaflet relative to the tissue engagement portions or clasps 5130, 5230, 5330. For example, when the clasp 5930 is open, the impedance between the electrode strips 5940, 5942 can be measured to determine leaflet insertion depth. When the clasp 5930 is closed, the impedance between each electrode strip 5940, 5942 and the electrode plate 5945 can be measured to determine leaflet capture depth. Advantageously, this configuration provides a continuous signal correlating to the amount of leaflet inserted while the clasp 5930 is open. Advantageously, this configuration confirms leaflet capture when the clasp 5930 is closed. Advantageously, this configuration can differentiate between different leaflet insertion scenarios (e.g., angled, askew, crooked, partial or under insertion, full insertion, over insertion, etc.) due at least in part to the configuration of the electrode strips 5940, 5942 along with the electrode plate 5945. For example, asymmetry in bioimpedance signals from the electrode strips 5940, 5942 can indicate that the tissue in the clasp 5930 is angled or askew. Similar principles apply to capture of other types of tissue.

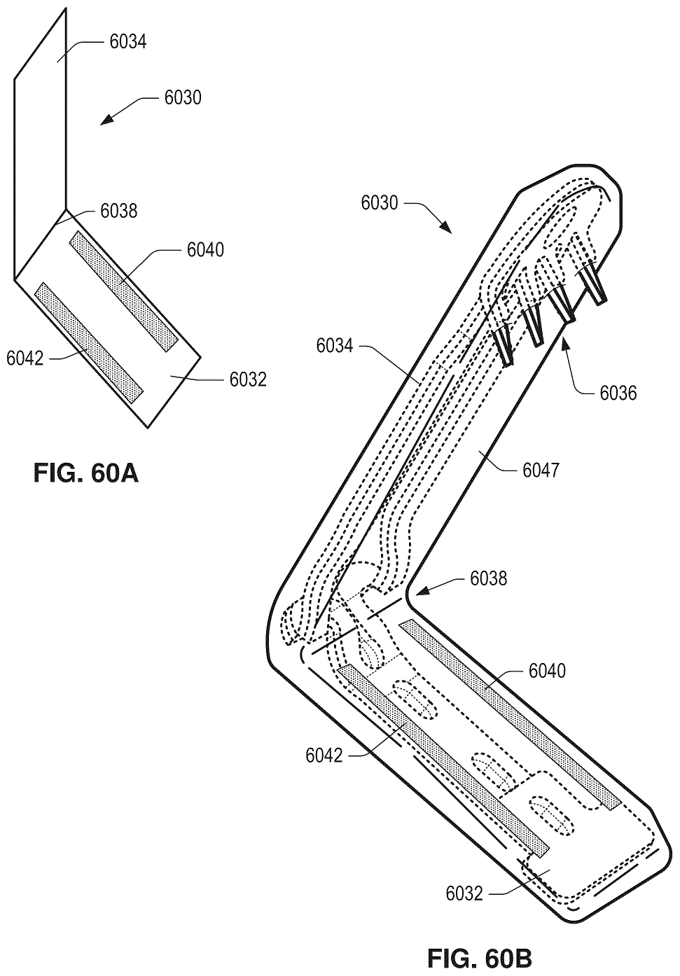

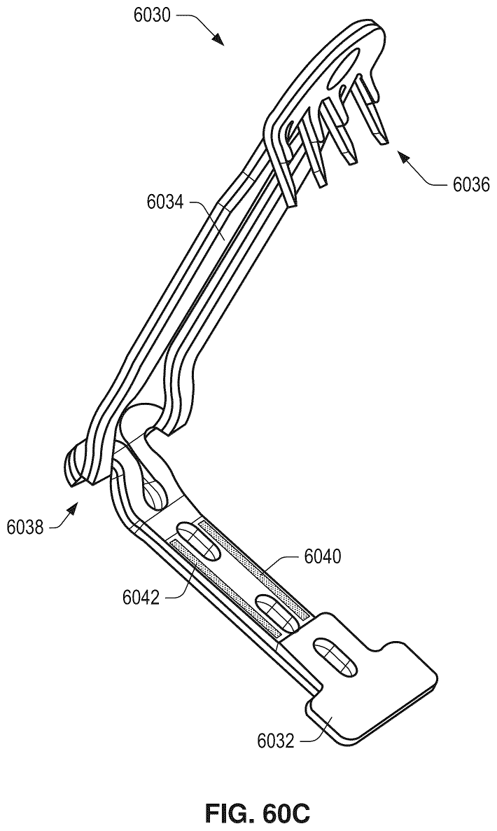

FIGS. 60A-62C illustrate an example tissue engagement portion or clasp 6030 with electrode strips 6040, 6042 and example bioimpedance signals from the example tissue engagement portion or clasp 6030. FIGS. 60A and 60B illustrate the tissue engagement portion or clasp 6030, which can be configured the same as or similar to the clasp 230 (or another tissue engagement portion or clasp herein), with a cover 6047 over the clasp 6030 (similar to the cover 240 of FIG. 25). FIG. 60C illustrates an example implementation of the tissue engagement portion or clasp 6030 of FIG. 60B without the cover 6047. It should be noted that each of the tissue engagement portions or clasps and associated bioimpedance-based components described herein can be implemented with or without a cover, an example of which is demonstrated by the clasp 6030 which is shown in FIG. 60B with the cover 6047 and in FIG. 60C without the cover.

The tissue engagement portion or clasp 6030 has electrode strips 6040, 6042 positioned on a first surface and/or first arm 6032 (e.g., over the portion of the cover 6047 that is over the first arm 6032). The tissue engagement portion or clasp 6030 can be implemented on any of the devices described herein. In addition, the tissue engagement portion or clasp 6030 can be the same as or similar to the clasps 130, 230, 330, 40856, 5030a, 5030b, 5030c, 5930 (or other tissue engagement portions) described herein and share many of the same components (e.g., arms 6032, 6034, joint portion 6038, means for securing 6036, and electrode strips 6040, 6042), properties, and functionality.

In the illustrated example of the tissue engagement portion or clasp 6030, the electrode strips 6040, 6042 run parallel to the length of the arm 6032 and cover a portion of the length of the arm 6032, with a separation between the electrode strips 6040, 6042 along a width of the arm 6032. In this example implementation of the tissue engagement portion or clasp 6030, the electrode strips 6040, 6042 are implemented over the cover 6047, but it should be noted that the electrode strips 6040, 6042 can be implemented below the cover 6047.

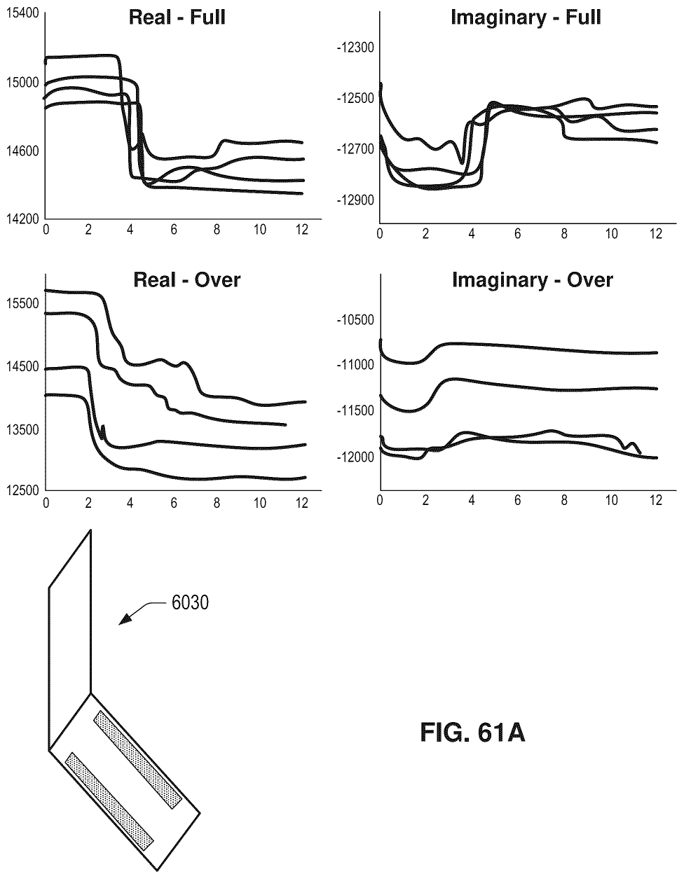

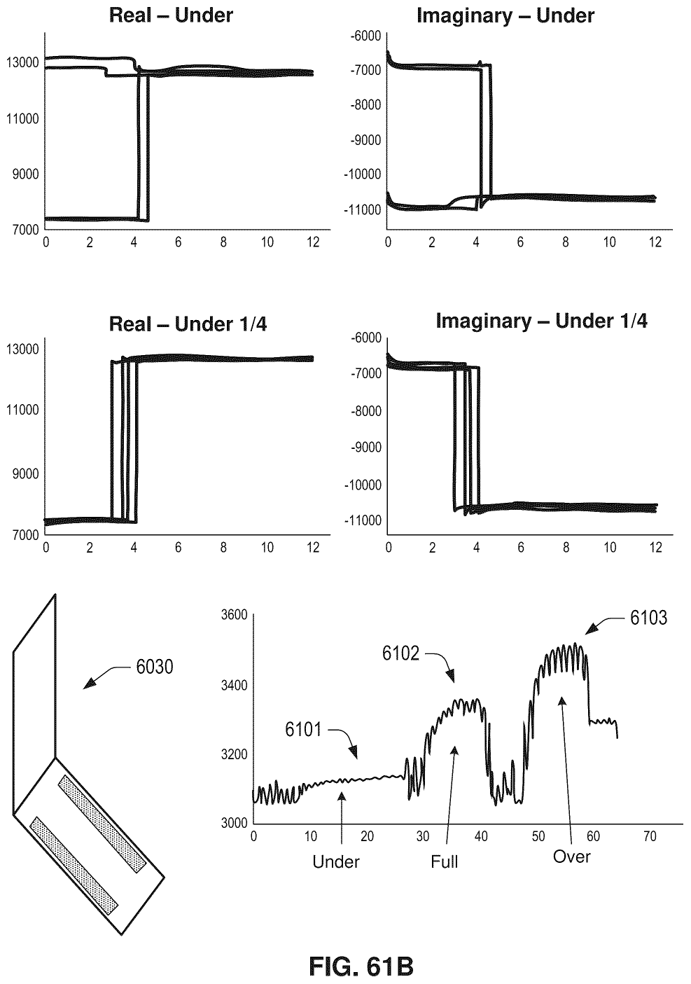

The top graphs of FIG. 61A illustrate the real (left graph) and imaginary (right graph) portions of the bioimpedance signal of the clasp 6030 when the leaflet is fully captured. The bottom graphs of FIG. 61A illustrate the real (left graph) and imaginary (right graph) portions of the bioimpedance signal of the clasp 6030 when the leaflet is over captured. The top graphs of FIG. 61B illustrate the real (left graph) and imaginary (right graph) portions of the bioimpedance signal of the clasp 6030 when the leaflet is under captured. The middle graphs of FIG. 61B illustrate the real (left graph) and imaginary (right graph) portions of the bioimpedance signal of the clasp 6030 when the leaflet is under captured, specifically when the leaflet is ¼ under captured. The bottom graph of FIG. 61B illustrates the real portion of the bioimpedance signal of the clasp 6030 when the leaflet is under captured (signal portion 6101), when the leaflet is fully captured (signal portion 6102), and when the leaflet is over captured (signal portion 6103). Asymmetry in bioimpedance signals from the electrode strips 6040, 6042 can indicate that the tissue in the tissue engagement portion or clasp 6030 is angled or askew. Similar principles apply to capture of other types of tissue.

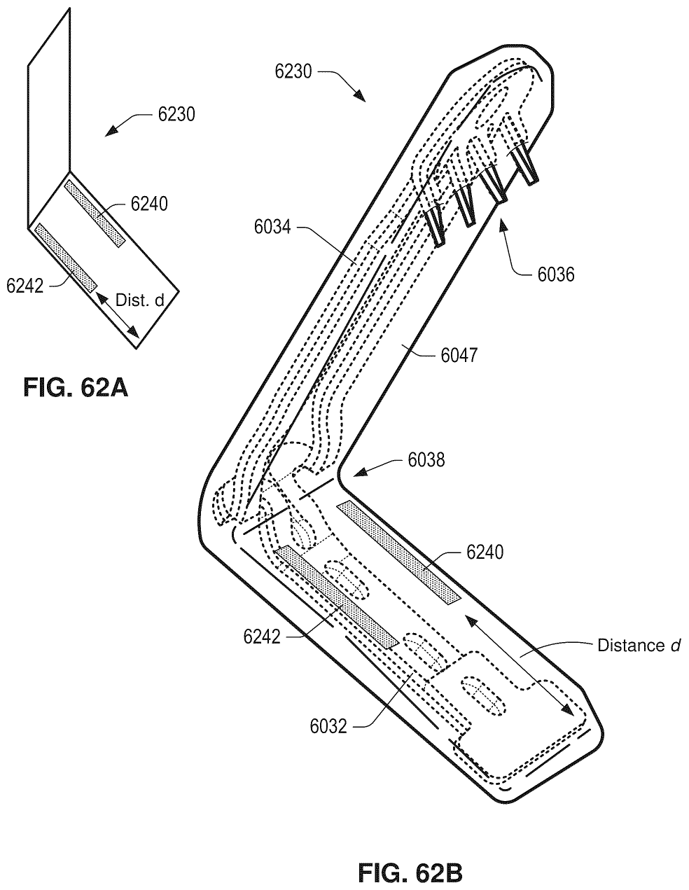

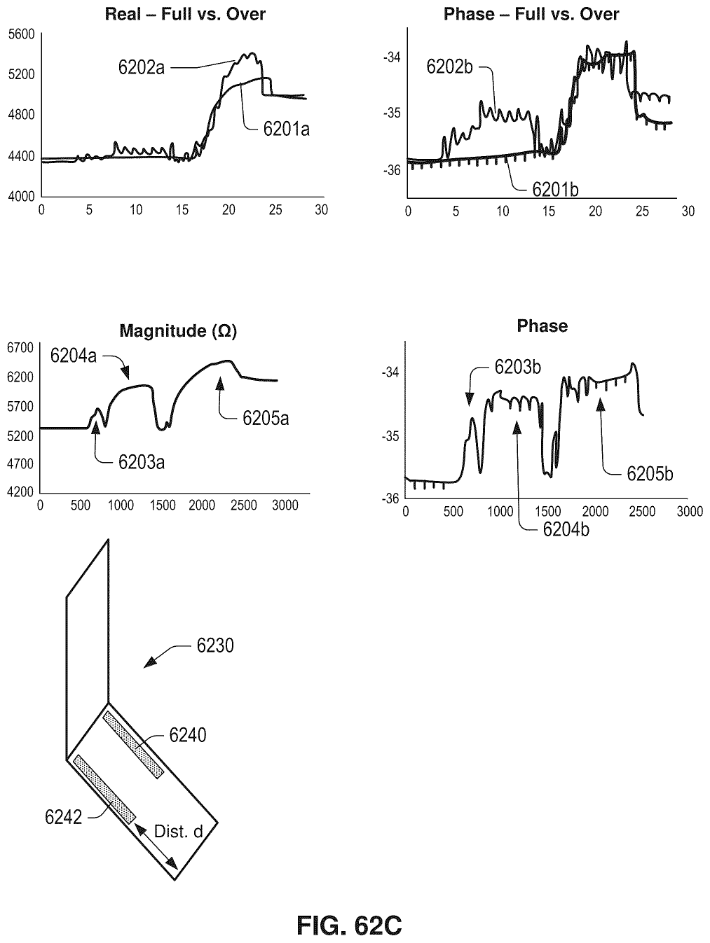

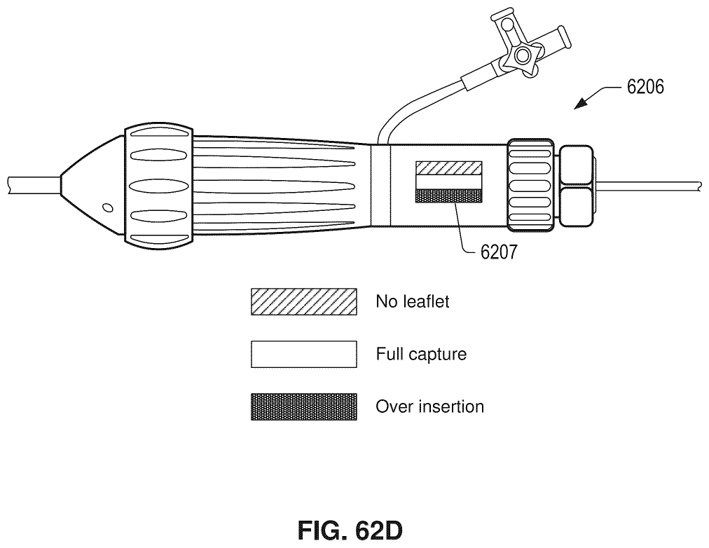

FIGS. 62A and 62B illustrate an implementation of a tissue engagement portion or clasp 6230 which can be the same as or similar to the tissue engagement portion or clasp 6030 of FIGS. 60A-C except that the electrode strips 6240, 6242 are offset from an edge (e.g., a free edge, an edge opposite a hinged end, etc.) of the surface and/or arm 6032 by a prescribed distance, d (e.g., about 6 mm). This offset changes the bioimpedance signal profiles for leaflet capture, as illustrated in the graphs of FIG. 62C. The top graphs of FIG. 62C illustrate the real (left graph) and phase (right graph) portions of the bioimpedance signal for a fully captured leaflet (signals 6201a, 6201b) and for an over-captured leaflet (signals 6202a, 6202b). The bottom graphs of FIG. 62C illustrate the magnitude (left graph) and phase (right graph) portions of the bioimpedance signal for two full insertions (signal portions 6203a, 6203b, 6204a, 6204b) and one over-insertion (signal portion 6205a, 6205b) on an ex vivo beating heart. In this example implementation of the clasp 6230, the electrode strips 6240, 6242 are implemented over the cover 6047, but it should be noted that the electrode strips 6240, 6242 can be implemented below the cover 6047. In some implementations, no cover is used.

Various algorithms can be implemented to analyze the bioimpedance signals of the tissue engagement portions or clasps 6030, 6230. These algorithms can include machine learning algorithms such as neural networks and other machine learning algorithms. For example, a feature-based random forest algorithm can be implemented that outputs an indication of an under, over, or fully captured leaflet when the tissue engagement portion or clasp 6030, 6230 is open and closed. This algorithm can be configured to determine if a leaflet is askew or angled in the tissue engagement portion or clasp 6030, 6230 and/or when only one electrode is covered. Advantageously, this provides indicators prior to closing the clasp 6030, 6230. As another example, another feature-based random forest algorithm can be implemented that outputs a continuous leaflet insertion indicator while the clasp 6030, 6230 is open and closed. This advantageously provides a larger amount of information to the user and can be configured to differentiate between captured targeted tissue (e.g., leaflet capture) and captured non-targeted tissue (e.g., chord capture).

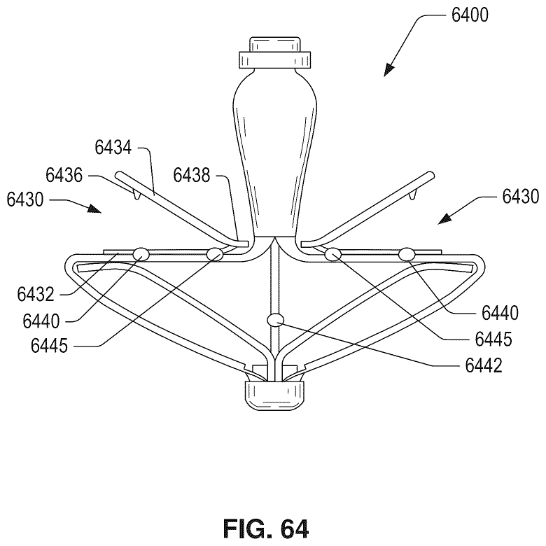

In some implementations, the tissue engagement portions or clasps 6030, 6230 can include a reference electrode (not shown). In some implementations, the reference electrode can be similar to the reference electrode described herein with reference to FIG. 64, e.g., the reference electrode can be part of the actuation element.

In some implementations, the reference electrode can be a dedicated reference electrode in the blood, an electrode on a catheter, or an external patch on the patient's skin. In some implementations, bio-impedance can be measured in three configurations: electrode A versus electrode B (e.g., electrode 6040 versus electrode 6042 or electrode 6240 versus electrode 6242), electrode A versus the reference electrode, and electrode B versus the reference electrode.

In some implementations, the reference electrode can be in measuring bioimpedance-based signals that can provide detailed information on tissue contact on the tissue engagement portion or clasp 6030, 6230 by comparing two unipolar measurements from each electrode (e.g., electrodes 6040 and electrode 6042 or electrode 6240 and electrode 6242). By way of example, if one electrode has a relatively high impedance indicating full insertion but the other electrode is indicating under insertion this means that the leaflet is inserted askew or at an angle. As another example, in a commissure area where mostly chordae are captured, each electrode 6040, 6042 or each electrode 6240, 6242 will show a different impedance but both impedances will be too low to be confused with full leaflet capture.

In some implementations, when the bipolar impedance is measured, the comparison between leaflet and no leaflet/blood is amplified due at least in part to the electrodes both touching leaflet and/or blood simultaneously. This provides a greater indication of leaflet insertion which may be more pronounced when the leaflet is inserted straight in the tissue engagement portion or clasp and the device or implant is perpendicular to the leaflet's free edge.

The electrode configuration of the example tissue engagement portion or clasp 6030 may be beneficial due at least in part to the electrode strips 6040, 6042 providing a continuous indication of leaflet insertion (which can be approximately linear) throughout the length of the tissue engagement portion or clasp 6030. This can be broken down into quantized signal regions indicating, for example, four categories of leaflet insertion: no leaflet, under insertion, full insertion, and over insertion. In some implementations, the average measurement of the electrode strips 6040, 6042 can be used to determine the label or category of leaflet insertion. The electrode configuration of the tissue engagement portion or clasp 6230 may be beneficial because there is little or no change in signal until the leaflet reaches the edge of the electrode strips 6242, 6240, which is already a distance, d, within the clasp 6230. The distance, d, can be configured to be an advantageous distance that indicates a sufficient insertion distance to achieve good leaflet capture. By way of example, the distance, d, can be about 6 mm or between about 4 mm and about 8 mm in certain implementations. Comparing this to the clasp 6030, the indication of leaflet insertion can be divided into 3 categories, combining the no leaflet insertion indicator with the under-insertion indicator because there may not be sufficient signal differentiation between no leaflet insertion and under insertion of leaflet with the clasp 6230.

In some implementations, a machine learning or other algorithm can be implemented that automatically determines the tissue or leaflet state based on the bioimpedance signals from the electrodes. For example, an algorithm can be implemented in conjunction with the clasp 6030 that interprets signals consistent with the signals 6101 of FIG. 61B as corresponding to no tissue/leaflet in the clasp, interprets signals consistent with the signals 6102 of FIG. 61B as corresponding to full tissue/leaflet insertion, and interprets signals consistent with the signals 6103 of FIG. 61B as corresponding to over insertion of the tissue/leaflet. The algorithm can also be used to generate an indicator for a user. For example, FIG. 62D illustrates a delivery system 6206 (similar to the delivery systems 102, 202 described herein) can include an indicator panel 6207 on a proximal end of the delivery system 6206. The indicator panel 6207 includes light or other indicators indicating no leaflet in the clasp, full leaflet capture, and over insertion of the leaflet. The user can visually check the indicator panel 6207 to determine the leaflet status without relying solely on echo imaging or other imaging techniques.

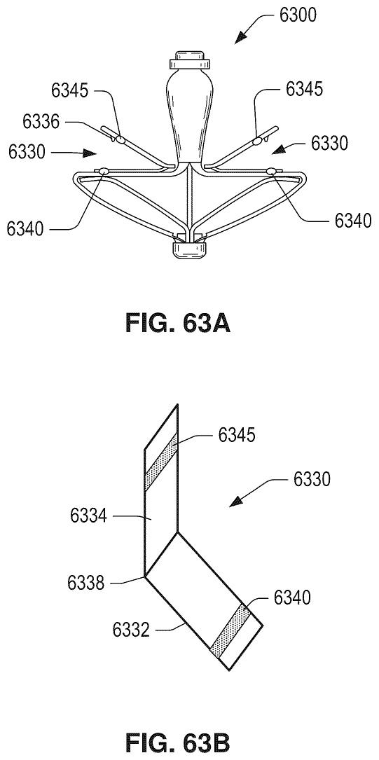

FIGS. 63A and 63B illustrate an example device 6300 with tissue engagement portions or clasps 6330 each having a first electrode 6340 positioned on a first surface and/or first arm 6332 and a second electrode 6345 positioned on a second surface and/or second arm 6334 of the tissue engagement portions or clasps 6330. The device 6300 can be the same as or similar to the devices 100, 200, 300, 8200, 8810, 40256, 5200 described herein. In addition, the tissue engagement portions or clasps 6330 can be the same as or similar to the clasps 130, 230, 330, 40856, 5030a, 5030b, 5030c (or other tissue engagement portions) described herein and share many of the same components (e.g., arms 6332, 6334, means for securing 6336, and joint portion 6338), properties, and functionality.

In the illustrated example of the tissue engagement portions or clasps 6330, there are two opposing electrodes 6340, 6345 coupled respectively to the first surface or first arm 6332 and to the second surface or second arm 6334 (or, in implementations without a second arm, to another portion of the device). Thus, the electrodes 6340, 6345 provide bioimpedance signals corresponding to different sides of a leaflet or other tissue. A benefit of this type of configuration is that the clasp 6330 can be configured to determine a thickness of the tissue between the electrodes 6340, 6345 and to scan the tissue thickness as the tissue passes through the clasp 6330 between the electrodes 6340, 6345. Moreover, this configuration of electrodes 6340, 6345 provides similar benefits to the clasp 5230 in that it can be configured to indicate that there is no leaflet or tissue capture when the clasp 6330 is closed due at least in part to the electrodes 6340, 6345 being shorted, or in contact with each other, resulting in the impedance value being drastically reduced relative to a configuration in which the electrodes 6340, 6345 are apart and/or in contact with tissue.

In some implementations, the first electrode can be coupled to an arm and the second electrode or opposing electrode can be coupled to another portion of the device (e.g., if no second arm is included).

By way of example, the opposing electrodes 6340, 6345 can be positioned on each side of the leaflet as it enters the tissue engagement portion or clasp 6330 and can effectively scan the leaflet as it passes over the electrodes 6340, 6345. The signals acquired as the tissue/leaflet passes between the electrodes 6340, 6345 can be used to generate a cross-sectional map of the thickness of the tissue/leaflet. In some implementations, these signals are acquired while the clasp 6330 is partially closed so that the electrodes 6340, 6345 are close to the tissue/leaflet.

In some implementations, the thickness of the leaflet tissue can be used by an operator to estimate or determine the strength of the leaflet. The thickness and strength of the leaflet can indicate how much tension force can be applied to the leaflet and/or whether the leaflet needs to be fully inserted into the clasp for secure capture. For example, stronger leaflet tissue can withstand higher forces and the barbs of the clasp 6330 can hold well in stronger tissue relative to weaker tissue. Advantageously, this may result in less stenosis and more coaptation.

FIG. 64 illustrates an example device 6400 with tissue engagement portions or clasps 6430 having electrodes 6440, 6445 similar to the device 5100 with tissue engagement portions or clasps 5130 of FIGS. 53A and 53B. The device 6400 can be the same as or similar to the devices 100, 200, 300, 8200, 8810, 40256, etc. described herein. In addition, the tissue engagement portions or clasps 6430 can be the same as or similar to the clasps 130, 230, 330, 40856, 5030a, 5030b, 5030c (or other tissue engagement portions) described herein and share many of the same components (e.g., arms 6432, 6434, means for securing 6436, and joint portion 6438), properties, and functionality.

In some implementations of the device 6400, there is an additional reference electrode 6442 implemented on the device 6400. This configuration can provide added sensitivity because the reference electrode 6442 is near the sensing electrodes 6440, 6445. The reference electrode 6442, or a similar reference electrode, can be implemented on any of the devices described herein to provide bipolar measurements of the bioimpedance. Bipolar configurations include measurement configurations where the sensing and reference electrodes are located in the same region, such as the heart. This can be compared to unipolar configurations where a reference electrode is located in a region different from the sensing electrode (e.g., where the sensing electrode is in the heart and the reference electrode is on the skin of the patient).

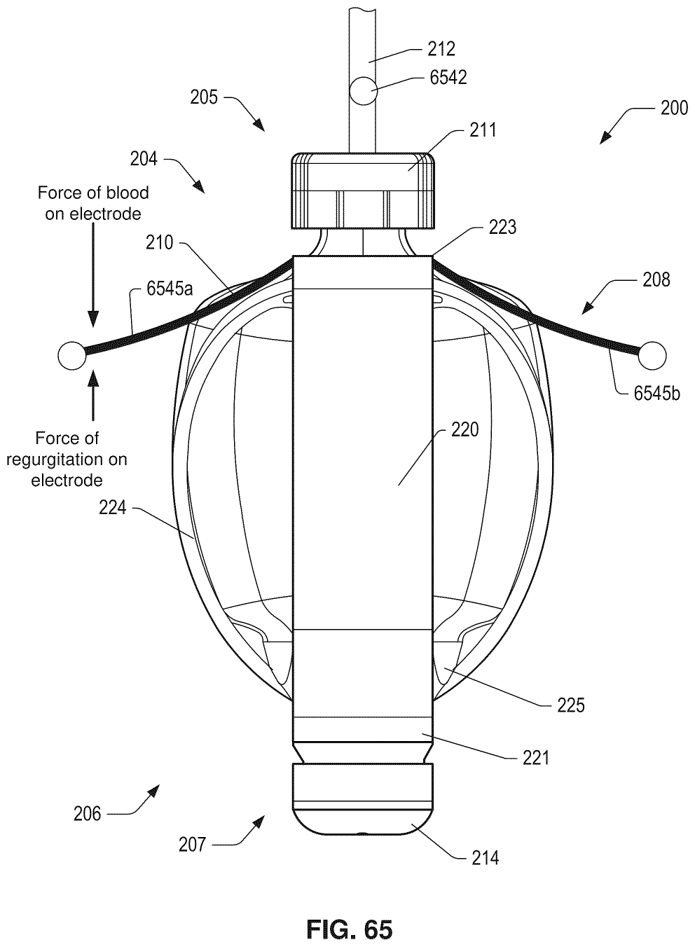

FIG. 65 illustrates an example of device 200 with the addition of flexible electrodes 6545a-b that protrude away from the device 200. The device 200 is described herein in greater detail with reference to FIGS. 22-37. The flexible electrodes 6545a-b are configured to measure blood flow and/or to detect leakage through the valve in which the device 200 is implanted. The flexible electrodes 6545a-b deflect as blood flows past them, with the amount of deflection being related to the differential pressure or flow. As the flexible electrodes 6545a-b deflect, the impedance relative to a reference electrode 6542 (e.g., on the actuation element 212) changes which can be used to determine the amount of deflection which in turn can be used to determine relative blood flow adjacent to the device 200. For example, regurgitation blood volume pushes the flexible electrodes 6545a, 6545b towards the atrium and closer to the reference electrode 6542, which causes the impedance to decrease. The amount of deflection (as measured by the change in impedance) can be used as a pressure sensor on either side of the device 200 such that the measurements can be used to determine whether there is a regurgitant volume adjacent to the device 200.

This is advantageous because a typical method for quantifying leaks in percutaneous procedures is through echo-based imaging. However, if there is not an echo of sufficient quality, it may be difficult to determine whether there is a leak after deploying the device 200. Many things may affect the quality of echo imaging including but not limited to the metal in the devices causing shadowing or ringing. Accordingly, bioimpedance measurements can be advantageous to provide an additional or alternative method for determining or monitoring leakage through the valve.

In some implementations, the device 200 can include flexible electrodes in addition to the flexible electrodes 6545a-b that are spread around the device 200 to monitor for leaks. The flexible electrodes 6545a-b can have a predetermined size and weight so that the force on the flexible electrodes 6545a-b can be determined based on the deflection of the electrodes 6545a, 6545b.

The amount of deflection can be determined using the bioimpedance measurements as described herein due at least in part to the flexible electrodes 6545a, 6545b acting similar to springs where force is proportional to deflection. Once the force or pressure of the blood is calculated, the flow rate can be calculated based on Bernoulli's equation. In some implementations, the regurgitant volume can also be calculated based on the size of the orifice (e.g., determined in echo).

In some implementations, forces on the device/implant can be determined using bioimpedance measurements. For example, the frame of the device can act as a spring. Prior to deployment and/or implantation, forces can be applied to the device and the deflection can be measured to determine the response of the device to known forces. In addition, impedance measurements can be made with the device deflected known amounts. Thus, a measured impedance can be related to a force on the device through the relationship of the opening distance of the device and measured impedance. The impedance value at baseline can be used to calibrate to the patient when the clasp is closed and then the change in baseline can be measured after the device is released to calculate the forces during systole and diastole. This measurement can be used for validation and testing during design and manufacturing or during deployment with forces indicating if there is a higher likelihood of single leaflet device attachment (SLDA). Moreover, the electrodes on the device can act as an implantable sensor that monitors forces during the life of the implant. Measurements can also be taken to determine the tension of the leaflet pulling the closed implant open. Such measurements can be used to predict leaflet damage or the risk of SLDA if the tension is sufficiently high. In addition, these measurements can correlate to stenosis, higher pressure gradients, and/or slippage of the leaflet during the implant closing as more tension is applied. Such measurements can also provide an indication of whether the device fully closes.

In addition, in implementations where forces are measured on either side of the implant, asymmetries can indicate asymmetrical tension on the leaflet, indicating that the delivery device is possibly warping the leaflets and when the device/implant is released the clinical results of the therapy could potentially change. Asymmetrical forces may be undesirable because it may cause changes in coaptation or other characteristics of the implanted device when the device is released from the delivery system. Symmetrical forces are desirable to reduce or eliminate changes in the performance of the device/implant after being released from the delivery system. Thus, being able to measure and/or monitor forces on the device/implant can be desirable.

### Example Configurations of Impedance Measurement Systems

As described herein, configuration and placement of electrodes on a device (e.g., on an anchor, tissue engagement portion, clasp, etc. of a device) can provide a number of different advantages as it applies to bioimpedance-based feedback measurements. However, electrical wiring of sensors and electrodes in a catheter may be difficult due at least in part to the limited space in the catheter. If it is desirable to include multiple sensors or electrodes, typical solutions would require running wiring for each electrode along the length of the catheter. However, the limited inner diameter of the catheter may restrict the number of wires that can be run from the proximal end of the catheter or other delivery system to the electrodes, thereby restricting the number of electrodes that can be used at the device or implant. This would then result in a reduction in the amount of information or the precision of the bioimpedance measurements acquired with the electrodes. Furthermore, increasing the number of electrical leads running to the device greatly complicates manufacturing the device and delivery system.

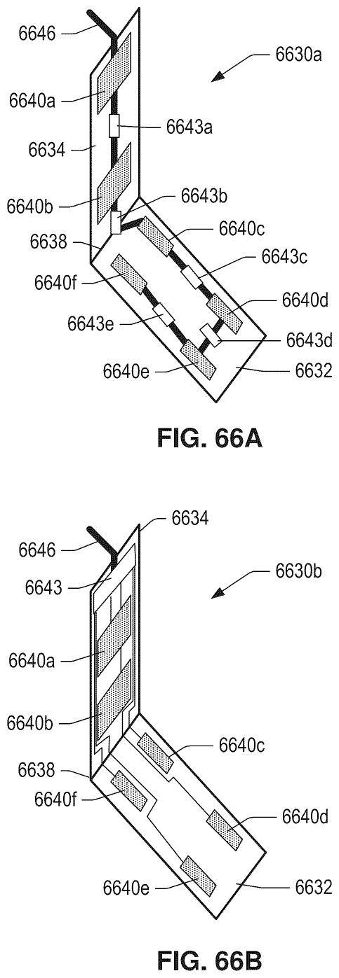

Accordingly, FIGS. 66A and 66B illustrate example electrode arrays that reduce the number of electrical leads required to enable the electrical leads to fit into small-lumen catheters. FIG. 66A illustrates an example tissue engagement portion or clasp 6630a, which can be the same as or similar to the clasps 130, 230, 330, 40856, 5030a, 5030b, 5030c (or other tissue engagement portions) described herein, with a series of electrodes 6640a-f coupled to the arms 6632, 6634 of the clasp 6630a with a joint portion 6638 coupling the arms 6632, 6634 to each other. One or more of the electrodes and/or electrode arrays can be optionally be implemented on other surfaces and/or portions of a device (e.g., not necessarily arms).

In some implementations, electrical lead 6646 couples the electrodes 6640a-f in series with one or more electrical components 6643a-e coupled in series between each electrode 6640a-f. In some implementations, the electrical lead 6646 then runs through the delivery device to deliver electrical signals to enable bioimpedance measurements.

In some implementations, electrodes are coupled in parallel, each electrode having electrical leads that electrically couple the respective electrode to a measurement system at a proximal end of the delivery system. In some implementations, the tissue engagement portion or clasp 6630a includes the electrical lead 6646 with electrical components 6643a-e coupled in series with the electrodes 6640a-f to enable individual measurement of each electrode without requiring electrical leads for each electrode. In some implementations, this is accomplished by using a resistor, capacitor, and/or inductor with a known and fixed value as the electrical component 6643a-e in series between each electrode 6640a-f. By using electrical components with different properties, the impedance measurements from each electrode can be individually determined using the electrical lead 6646. For example, even with the electrodes 6640a-f coupled in series, the measured bioimpedance values can be separated because the current through the electrical lead 6646 is known as well as its frequency. From the impedance measured, it is possible to calculate resistance, capacitance, and/or inductance and subtract the known value of the electrical component that is inserted in series. As a result, it is possible to determine the measured impedance from the electrode as if it were coupled in parallel.

FIG. 66B illustrates an example clasp 6630b with a plurality of electrodes 6640a-f and an analog-to-digital converter (ADC) chip 6643 coupled to each electrode 6640a-f. The ADC chip 6643 is configured to convert the signals from the electrodes 6640a-f into a digital signal that can be sent over the electrical lead 6646 using digital packets, thereby separating out each electrode signal using digital data transfer protocols.

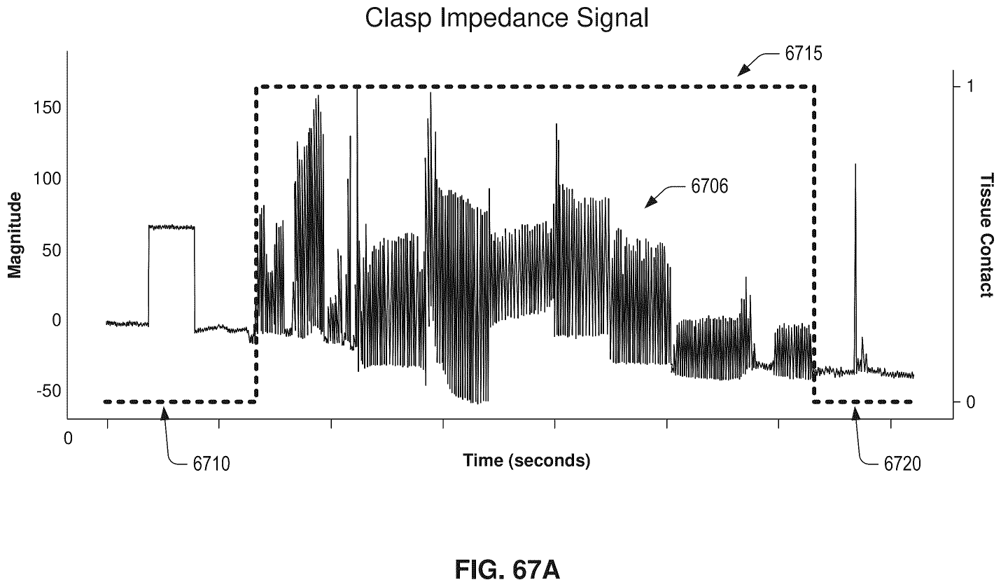

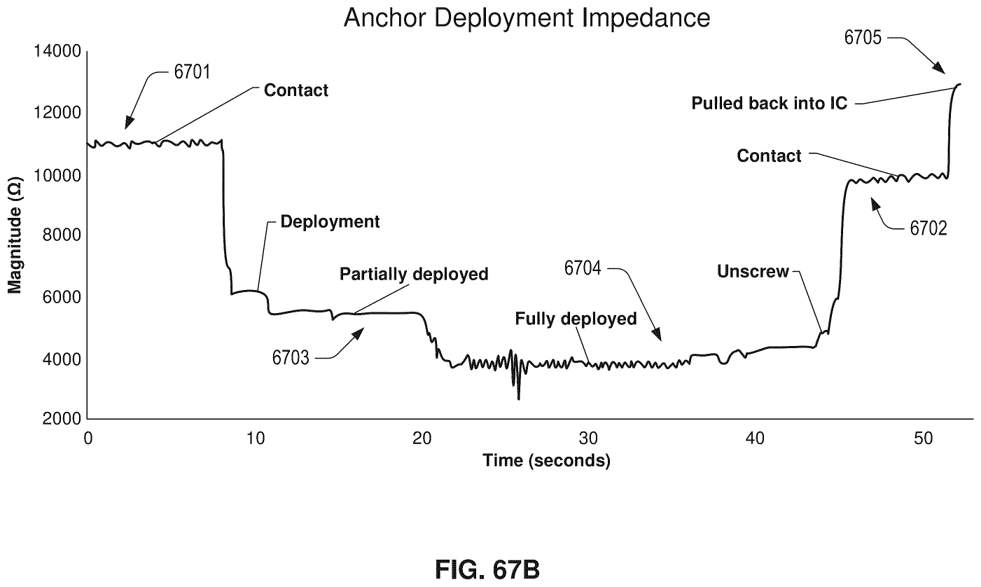

FIG. 67A illustrates an example of a bioimpedance signal 6706 with oscillations corresponding to diastole and systole of the heart. Changes in contact between the electrodes and the tissue arising with fluctuations caused by the beating of the heart may result in an oscillation in the measured bioimpedance signal 6706. A signal processing algorithm can be implemented that uses the oscillations (e.g., the peak-to-peak amplitude of the bioimpedance signal) and the mean value of the bioimpedance signal to determine a tissue state relative to a clasp or anchor. For example, when there are high peak-to-peak oscillations it can be determined or concluded that there is not ideal contact between the clasp and the tissue. As more tension is applied, the tissue makes better contact with the tissue engagement portion (e.g., anchor, clasp, etc.) and the peak-to-peak oscillations are reduced while the magnitude of the average signal increases. This can be used to generate a binary determination, for example, of no tissue contact in time period 6710, tissue contact during time period 6715, and no tissue contact during time period 6720.

The bioimpedance concepts described above with respect to tissue capture can also be applied to a variety of medical systems, devices, and procedures. In some implementations, the bioimpedance concepts described herein can also be applied to anchor deployment of anchors of a variety of medical systems and devices in a variety of medical procedures. For example, annuloplasty procedures (e.g., a reduction of the annulus) can benefit from the measurement of bioimpedance signals. Various indications of anchor placement and/or tissue engagement would be valuable and improve procedures and safety.

In some implementations, as anchors of a transcatheter annuloplasty system/device are implanted at or around an annulus, bioimpedance signals can be monitored to determine the deployment status of each anchor and/or an associated device, such as an annuloplasty implant.

FIG. 67B illustrates an example of a bioimpedance signal as a delivery device implants a tissue anchor (e.g., a helical tissue anchor, a dart-like anchor, a hook-like anchor, etc.) at an annulus of a native valve. The bioimpedance signal indicates contact with tissue (signal portions 6701, 6702), partial and full deployment or insertion in tissue (signal portions 6703 and 6704, respectively), and removal of the delivery device (signal portion 6705). In some implementations, the bioimpedance signal represents a single anchor being deployed or inserted in tissue and the process can be repeated for each anchor to be deployed (e.g., 5-25 anchors, 10-20 anchors, 12-17 anchors, etc.) sequentially (or if the anchors are deployed simultaneously, each anchor can be analyzed at the same time). In some implementations, the bioimpedance signal can indicate a situation in which all anchors are electrically shorted together.

In some implementations, the disclosed medical systems, devices, and procedures utilize one or more anchors (e.g., helical anchors, darts, hooks, clasps, clamps, barbs, arms, etc.). Individual anchors can include one or two, or more than two electrodes. An electrical signal can be provided to the electrodes and one or more electrical sensors can be configured to measure various electrical signals, including bioimpedance signals. As an anchor is implanted in tissue, the bioimpedance signal decreases, similar to a short circuit. Ex vivo measurements can be made, and these measurements can be used to determine an anchor depth based on or in response to the electrical signals from the anchors being deployed in vivo. Thus, anchor depth can be determined based on the ex vivo measurements and based on the electrical signals from the anchor as it is being implanted. This is bolstered by the change in impedance as the anchor moves from blood to tissue. Thus, indicators can be determined and provided to a user to indicate when tissue has been contacted and the depth of penetration in tissue of an anchor. Indicators can be configured to indicate anchor deployment status, the anchor deployment status including the anchor in contact with tissue, a partially deployed anchor, and a fully deployed anchor. In some implementations, amplitude modulation can be used when considering the length of DFT wire connecting between anchors as a resistor. This can be used to monitor consecutive anchor deployment.

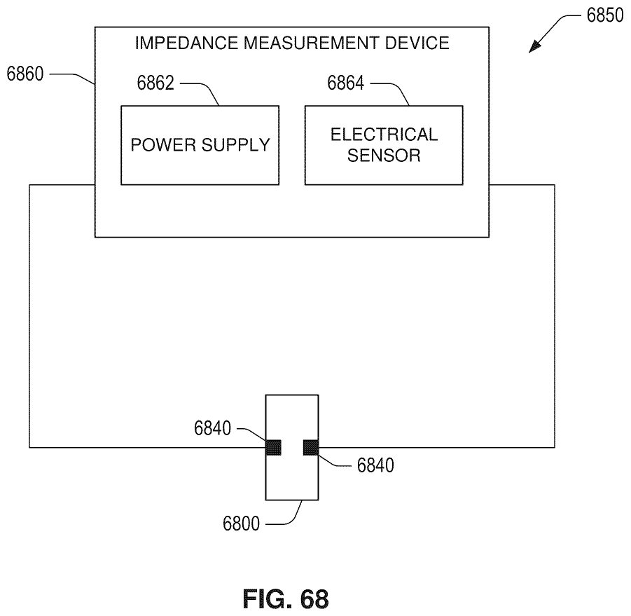

In some implementations, an anchor with two electrodes is configured to facilitate monitoring of bioimpedance. This can be done to monitor anchoring of the anchor to indicate a successful and/or complete penetration of the anchor into the tissue. An impedance measurement device (e.g., the impedance measurement device of FIG. 68) can be coupled to a proximal end of an anchor drive or anchor driver. The impedance measurement device can be configured to use a bipolar connection in a way that the positive and negative leads are isolated from each other but still located in the same region (e.g., the heart). This configuration can provide added sensitivity because the reference electrode is near the sensing electrode. Advantageously, this reduces noise relative to systems that measure electrical signals through an expanse of tissue (e.g., relative to a unipolar configuration). Algorithms can be implemented with smart thresholds that can be applied to the electrical signals from the impedance measurement device. The algorithms can generate real time indicators that can be provided to a user to indicate when a catheter or anchor is in contact with the tissue and/or partially or fully deployed in the tissue. It should be noted that the electrical measurements described herein can be unipolar (e.g., with an electrode on the skin or far away from the measurement site) or bipolar (e.g., where a reference electrode is in close proximity to the measurement electrode).

FIG. 68 illustrates an example bioimpedance signal measurement system 6850 that includes a device 6800 (e.g., an implantable device, a delivery device, a treatment device, etc.) and an impedance measurement device 6860. The impedance measurement device 6860 can include a power supply 6862 and an electrical sensor 6864. The device 6800 can include electrodes 6840 configured to receive electrical power from the power supply 6862. Wiring connects the electrodes 6840, the power supply 6862, and the electrical sensor 6864. The device 6800 can be any of the devices described herein such as the devices 100, 200, 300, 5100, 5200, 5300, 6300, 6400, 8200, 8810, an annuloplasty implant, a stent, a valve, a prosthetic valve, a delivery device, an anchor driver, a chordae repair device, or the like.

In some implementations, the electrodes 6840 are coupled to one or more anchors of the device 6800. In some implementations, the electrodes 6840 are coupled to clasps, such as the clasps 130, 230, 330, 40856, 5030a, 5030b, 5030c (or other tissue engagement portions) described herein.

The electrical sensor 6864 is configured to measure electrical signals, such as bioimpedance signals, voltage, current, etc. from the electrodes 6840. The electrical sensor 6864 can be configured to measure other electrical properties such as, for example and without limitation, resistance, inductance, capacitance, voltage, current, components of impedance, and the like. The bioimpedance measurements (as well as resistance, inductance, capacitance, voltage, and/or current readings) acquired by the electrical sensor 6864 can be different based on the anatomy or anatomies that the indicator electrodes 6840 are near or in contact with. Thus, the electrical characteristics measured by the electrical sensor 6864, in particular the bioimpedance signals, can be used to determine the relative locations of a clasp, anchor, other device components, etc. and anatomy (e.g., tissue, etc.) that the device is in contact with, as described herein. For example, the value of the bioimpedance signal and/or changes in bioimpedance can signal that the electrodes are in blood, contacting tissue (e.g., leaflets), differentiating tissue (e.g., leaflet tissue versus chord tissue), transitioning from being primarily in contact with blood to being partially or primarily in contact with tissue, and/or transitioning from being partially or primarily in contact with tissue to being primarily in contact with blood.

The power supply 6862 and the electrical sensor 6864 can be separate devices or combined in a single device. The power supply 6862 can be configured to provide alternating current to the device 6800. The electrical sensor 6864 can take a variety of different forms, including an impedance meter. By controlling the alternating current and measuring the voltage, the impedance can be calculated. The impedance can be used to determine the position of the electrodes with respect to targeted tissue (e.g., an annulus of a valve, a leaflet, etc.). The impedance measurement device 6860 can implement any of the algorithms described herein to indicate a status of the device 6800 or component thereof (e.g., clasps or anchors) that can include, full capture of a leaflet, under capture of a leaflet, over capture of a leaflet, a relative position of a leaflet in the clasp, a status of the clasp (e.g., open, closed, etc.), status of an anchor (e.g., partially deployed, fully deployed, in contact with tissue, etc.), or any combination of these and the like. The algorithms can include machine learning algorithms such as neural networks, decision tree algorithms, random forest algorithms, threshold-based algorithms, and the like. Thus, the impedance measurement device 6860 can include one or more processors and non-volatile memory configured to store and to execute the one or more algorithms to determine targeted quantities based at least in part on the measurements provided by the electrical sensor 6864. In some implementations, the derived indicators from the impedance measurement device 6860 can be displayed or otherwise provided to a user or a partially- or fully-automated system to provide bioimpedance-based feedback for medical procedures.

Removing Impedance Measurement Sensors from Device

As described herein, it is advantageous to use bioimpedance-based feedback in medical procedures, such as implanting a device in a valve. The bioimpedance-based feedback can be used to determine leaflet insertion, for example. To do so, in some implementations, electrodes or sensors are coupled to devices (e.g., anchors, tissue engagement portions, clasps, etc. of the devices) disclosed herein with electrical leads leading from the electrodes to a proximal end of the delivery system to enable acquisition and measurement of bioimpedance signals. However, it can also be desirable to disconnect the electrodes from the electrical leads or to remove the electrodes (or sensors) and electrical leads after implantation of the device (e.g., so electrical wires are not active in the implant after the procedure). Accordingly, disclosed herein are methods and devices to facilitate removal and disconnection of electrical leads from electrodes on the device. In addition, disclosed herein are methods and devices to remove electrodes or sensors from the device after it has been implanted. In addition, disclosed herein are methods and devices that enable connection of a flexible PCB (which can comprise the electrode(s) and/or other sensor(s) of various implementations described herein) to the device such that it can be removed easily in a transcatheter procedure from the proximal side of the delivery system (e.g., a catheter).

The use of PCBs (including flexible PCBs) is advantageous because it allows detailed designs in both the shape and the number of electrodes. A PCB can comprise an array of electrodes which further enables the acquisition of many bioimpedance measurements. With a larger amount of measurements and data, machine learning and other such algorithms can be used that provide more useful and accurate indicators associated with the implantation process (e.g., leaflet capture, leak detection, force monitoring, etc.). Furthermore, the non-conductive portions of the PCB can be designed to achieve certain purposes, such as to enable the removal of the PCB as part of the implantation process. This may be advantageous as well because there are challenges associated with making flexible PCBs biocompatible, meaning that it may be undesirable to leave flexible PCBs in a patient as part of the device.

While the examples shown and described herein often focus on the example of a flexible PCB, other arrangements, configurations, arrays, sensors, electrodes, wires, leads, lines, etc. can be used and/or connected in similar ways, even if a PCB is not itself used (i.e., while a PCB is advantageous, the concepts herein do not require a PCB, even if PCBs are used as an example in various implementations herein).

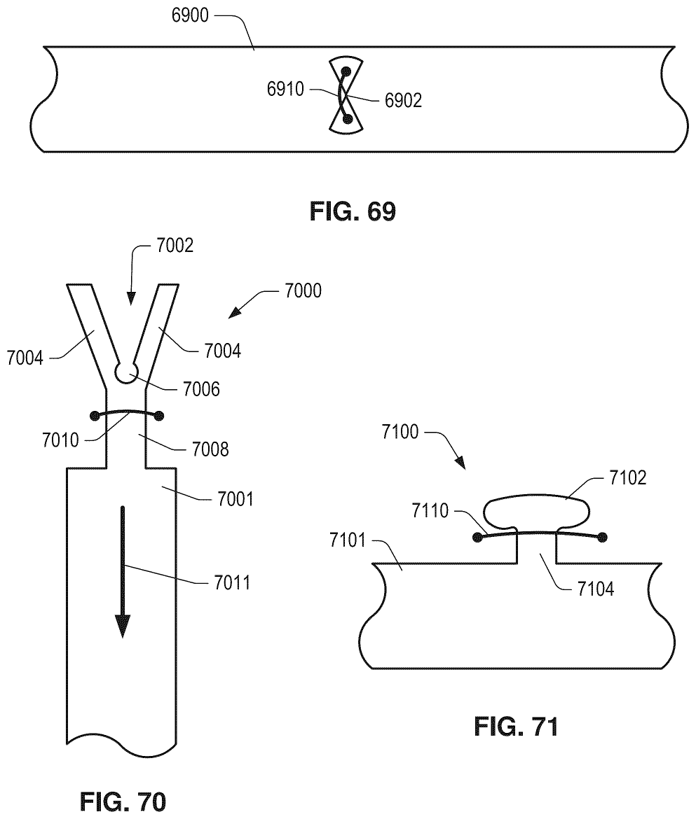

FIGS. 69-74 illustrate a variety of configurations of connecting a flexible PCB, electrode, electrode array, sensor, etc. to a device that allow for easy removal of the flexible PCB, electrode, electrode array, sensor, etc. FIG. 69 illustrates a portion of an example flexible PCB 6900 with a stress concentration point 6902 in the flexible PCB 6900. A suture 6910 can be looped over the stress concentration point 6902 to secure the flexible PCB 6900 to a device (such as the devices 100, 200 described herein). The stress concentration point 6902 is configured so that a relatively small applied force will cause the stress concentration point 6902 to tear. With the stress concentration point 6902 torn, the flexible PCB 6900 can be removed because the suture 6910 is no longer securing the PCB to the device although the suture 6910 remains secured to the device.

FIG. 70 illustrates a portion of a flexible PCB 7000 with a Y-shaped protrusion 7002 extending from one end of the flexible PCB 7000. The protrusion 7002 includes legs 7004 with a rotation cutout 7006 configured to enable the legs 7004 to rotate toward one another when a force is applied to the legs 7004. A suture 7010 is looped over a bridge portion 7008 of the protrusion 7002 to secure the flexible PCB 7000 to a device. When the flexible PCB 7000 is pulled in the direction of the arrow 7011, the protrusion 7002 moves down toward the suture 7010. With the application of sufficient force, the legs 7004 rotate toward one another to allow the flexible PCB 7000 to be detached from the device while the suture 7010 remains attached to the device. The wider portion 7001 of the flexible PCB 7000 makes it so only a force in the direction indicated by the arrow 7011 will cause the suture 7010 to pass over the protrusion 7002 to free the flexible PCB 7000 from the device.

FIG. 71 illustrates a portion of a flexible PCB 7100 with a round protrusion 7102 extending from a body 7101 of the flexible PCB 7100. A suture 7110 is looped over a neck portion 7104 of the round protrusion 7102 to secure the flexible PCB 7100 to a device. The round protrusion 7102 can be configured to fold or wrap over a side of a device (e.g., around a side of a clasp 130, 230). By pulling away from the device (e.g., out of the plane of the figure), the round protrusion 7102 allows the suture 7110 to pass over the rounded portion to free the flexible PCB 7100 from the device while allowing the suture 7110 to remain affixed to the device.

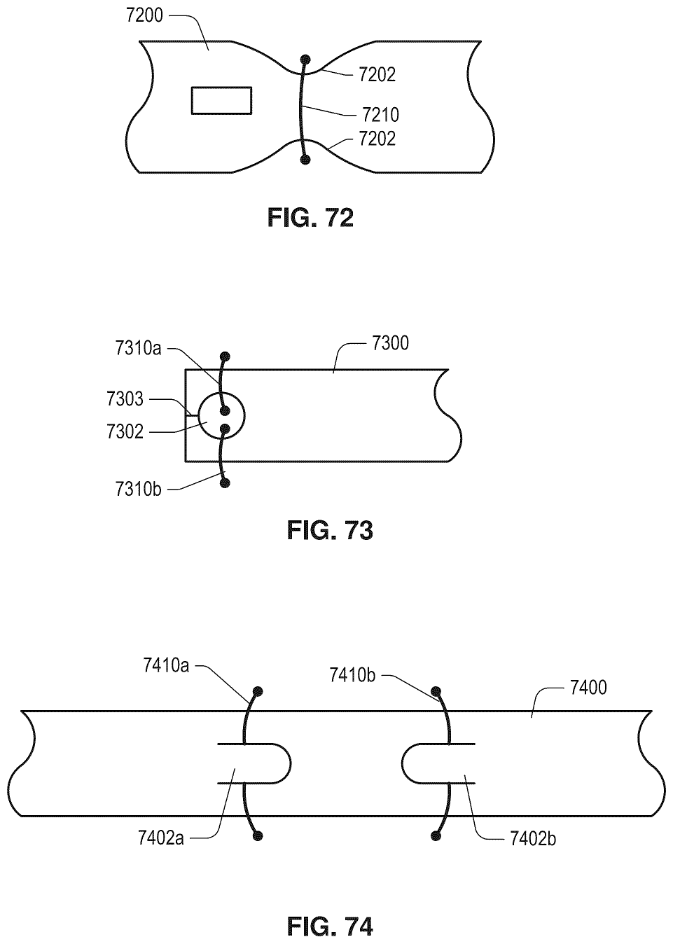

FIG. 72 illustrates a portion of a flexible PCB 7200 with side indents 7202 to facilitate securing the suture 7210 over the flexible PCB 7200 and to the device to secure the flexible PCB 7200 to the device. The side indents 7202 are configured to provide a positive lock in a targeted location of the flexible PCB 7200 so that the suture 7210 does not interfere with measurements by electrodes incorporated into the flexible PCB 7200. The size and configuration of the side indents 7202 affects the amount of force required to pull the flexible PCB 7200 out from under the suture 7210. Pulling parallel to the length of the flexible PCB 7200 causes the flexible PCB 7200 to pass under the suture 7210 so that it can be removed while allowing the suture 7210 to remain affixed to the device.

FIG. 73 illustrates a portion of a flexible PCB 7300 forming a hole 7302 (e.g., a circular hole) with a relief 7303. The flexible PCB 7300 can be secured to the device using one or more sutures 7310a, 7310b. The relief 7303 is cut through the end of the flexible PCB 7300 so that the sutures 7310a, 7310b can exit the hole 7302 when a force is applied to pull the flexible PCB 7300 away from the end with the hole 7302. In some implementations, the relief 7303 passes only partially from the hole 7302 to the end of the flexible PCB 7300. The length of this bridge can be configured to tailor the force required to remove the flexible PCB 7300 from the device.

FIG. 74 illustrates a portion of a flexible PCB 7400 that forms a pair of bi-directional tongues 7402a, 7402b. 6. The bi-directional tongues 7402a, 7402b form tabs that allow sutures 7410a, 7410b to pass under to secure the flexible PCB 7400 to a device. This configuration facilitates assembly of the device with the flexible PCB 7400 because the sutures 7410a, 7410b can be implemented in the fabric implant cover and then the flexible PCB 7400 can be added with the sutures 7410a, 7410b being woven through the tongue flaps of the bi-directional tongues 7402a, 7402b to lock the flexible PCB 7400 to the device. In some implementations, the depth of the tongue flaps affects the force required to pull the PCB out. It should be noted that for each of the flexible PCBs described herein with reference to FIGS. 69-74, the flexible PCB can be attached over a cover of the device (e.g., the sutures attach to the cover) or under a cover of the device (e.g., the sutures attach to the frame). However, a cover is not required in any implementation, and the flexible PCB can be attached to the device in a variety of ways.

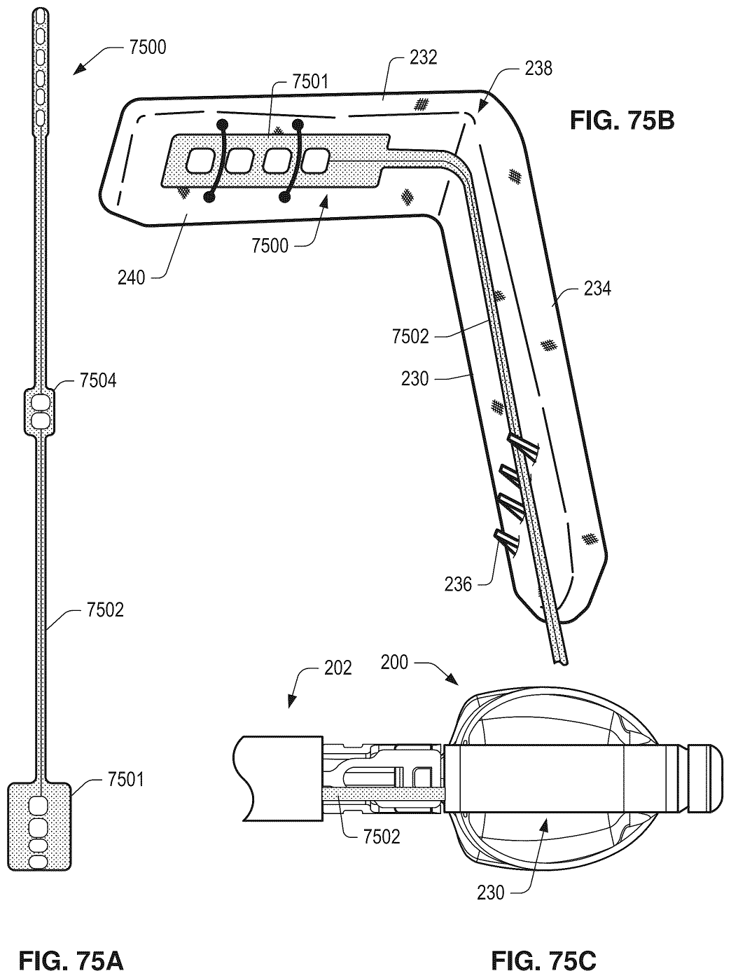

FIGS. 75A, 75B, and 75C illustrate a PCB 7500 that is configured to be pulled through the barbs 236 of the device 200 (or any other device described herein, such as the device 100) to remove the PCB 7500 from the device 200. The PCB 7500 is a flexible PCB and includes an electrode pad or electrode array 7501 comprising one or more electrodes at a distal end of the PCB 7500. The PCB 7500 includes leads 7502 extending proximally from the electrode pad/array 7501. The PCB 7500 can also include a reference electrode 7504. The leads 7502 are configured to extend from the electrode pad/array 7501 to a proximal end of the delivery system 202.

In some implementations, the PCB 7500 is secured to the tissue engagement portion or clasp 230, either to the frame of the clasp 230 or to the cover 240 covering the tissue engagement portion or clasp 230. When installed on the device 200, the leads 7502 can extend between (or around) optional friction enhancing elements or barbs 236 of the tissue engaging element or clasp 230. Pulling on a proximal end of the leads 7502 causes the electrode pad/array 7501 to pass between the friction enhancing elements or barbs 236 before entering a lumen of the delivery system 202 to be removed from the patient as part of the process of implanting the device 200.

In some implementations, the space between barbs 236 is about 0.8 mm and for an electrode pad/array 7501 that is about 1 mm wide, it requires a force of around 1 N to remove the PCB 7500 from the device 200. For an electrode pad/array 7501 that is about 1.5 mm wide, a force of around 1.5 N is required to remove the PCB 7500 from the device 200.

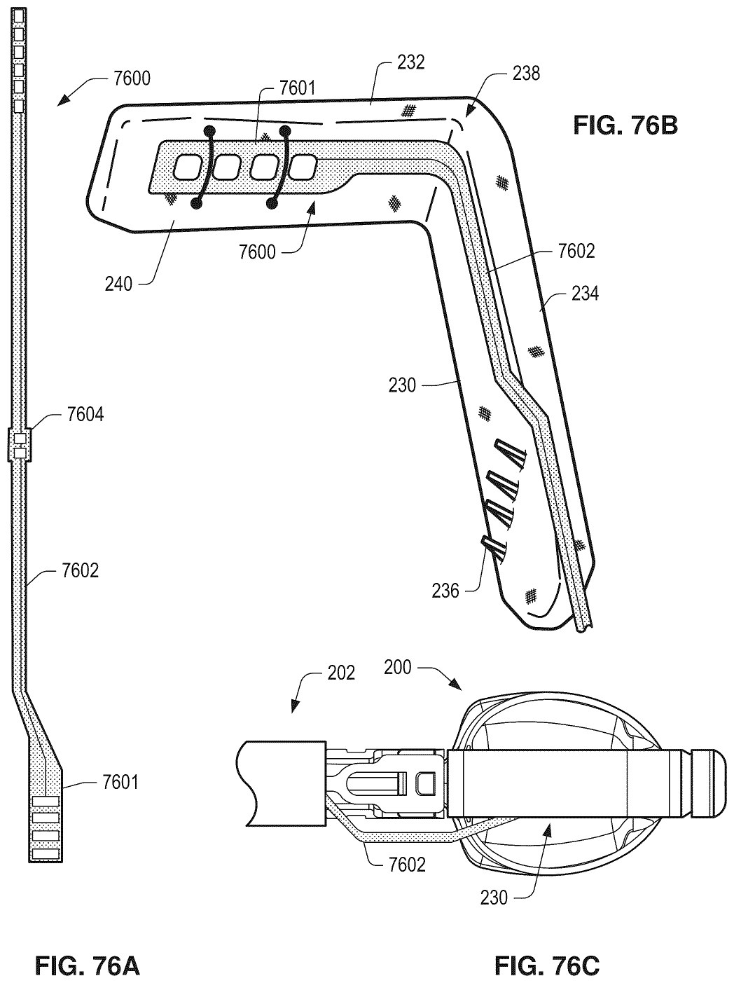

FIGS. 75A-75C illustrate an example of routing the leads 7502 through the barbs 236 but it should be noted that the leads 7502 can be routed differently than illustrated. The leads 7502 can be routed out of the clasp 230 at any point. For example, the leads 7502 can be routed out of the side of the clasp 230 near optional friction enhancing elements or barbs 236 (similar to what is illustrated in FIGS. 76B and 76C). In some implementations, the leads 7502 can be routed out of the side of the tissue engagement portion or clasp 230 at some point along a first arm or a movable arm 234, e.g., at or near a joint portion 238, at or near a connection point between a movable arm and another portion of the device, e.g., a fixed arm, a paddle, a coaptation element, etc. In some implementations, the leads 7502 can be routed out of the side of the tissue engagement portion or clasp at some point along a second arm or a fixed arm 232 (or if no first arm or fixed arm is used, a corresponding portion of the device).

In some implementations, the leads 7502 can be routed around the first arm or movable arm 234 (and/or around an optional fixed arm 232 if a fixed arm is included in the device, or around another component) to a side opposite the grasping side (e.g., a non-grasping side or a side opposite the side that includes the barbs 236). This can include routing the leads 7502 through a cloth or cover if the clasp 230 includes a cover.

In some implementations, the leads 7502 can be routed to avoid interacting with tissue captured, gripped, contacted, or clasped by the friction enhancing elements or barbs 236. This can be advantageous to avoid interfering with capturing of tissue by the clasp 230. In certain implementations, an opening can be maintained in the clasp 230 to facilitate removal of the leads 7502.

FIGS. 76A, 76B, and 76C illustrate another PCB 7600 that is configured to be pulled and exit through a side of an example tissue engagement portion or clasp 230, around the barbs 236 of the device 200 (or any other device described herein, such as the device 100) to remove the PCB 7600 from the device 200. The PCB 7600 is a flexible PCB and includes an electrode pad or electrode array 7601 comprising one or more electrodes at a distal end of the PCB 7600. In some implementations, the electrode pad/array 7601 is offset laterally relative to leads 7602 extending proximally from the electrode pad/array 7601. In some implementations, the PCB 7600 can also include a reference electrode 7604.

In some implementations, the leads 7602 are configured to extend from the electrode pad/array 7601 with a change in direction to a proximal end of the delivery system 202. In some implementations, the change in direction is configured to cause a pulling force on the leads 7602 to cause the electrode pad/array 7601 to exit the clasp by going around the barbs 236, using the barbs 236 as a fulcrum.

In some implementations, the PCB 7600 is secured to the tissue engagement portion or clasp 230, either to the frame of the clasp 230 or to the cover 240 covering the clasp 230. In some implementations, when installed on the device 200, the leads 7602 extend around friction enhancing elements or barbs 236 of the clasp 230. Pulling on a proximal end of the leads 7602 causes the electrode pad/array 7601 to pass around the friction enhancing elements or barbs 236 before entering a lumen of the delivery system 202 to be removed from the patient as part of the process of implanting the device 200. The PCB 7600 can be configured to exit the clasp 230 at or near a first arm 234 (e.g., a moveable arm), at or near a second arm 232 (e.g., a fixed arm, a paddle, etc.), at or near the joint portion 238, and/or at or near friction enhancing elements or barbs 236.

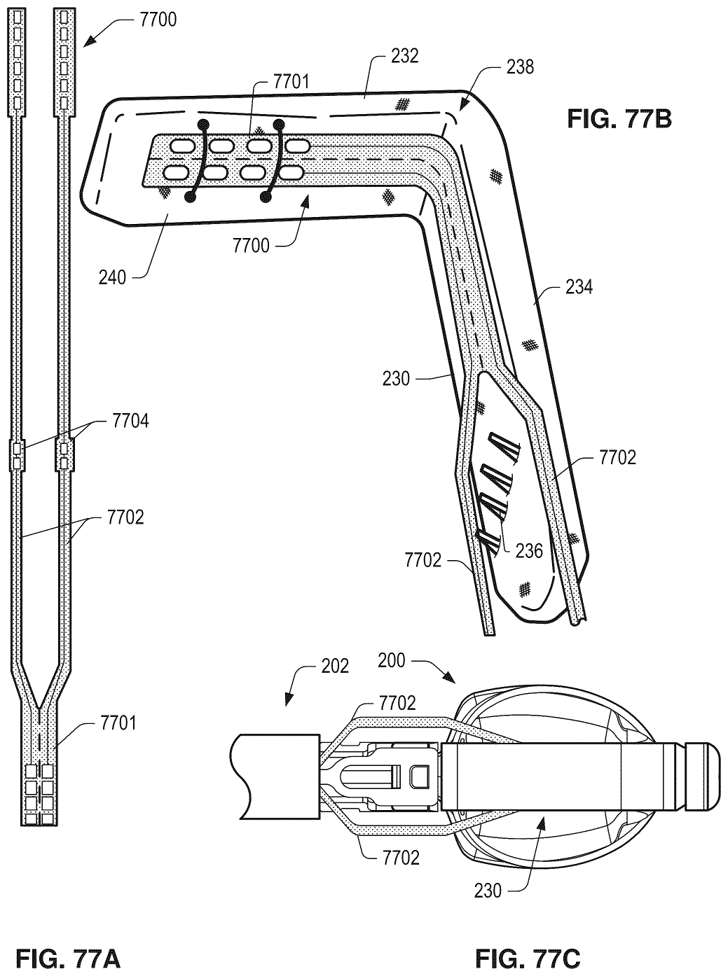

FIGS. 77A, 77B, and 77C illustrates another PCB 7700 that is configured to be split apart when pulled so that half exits through one side of the clasp 230 and the other half exits through the other side of the clasp 230, each half exiting the clasp around the friction enhancing elements or barbs 236 of the device 200 (or any other device described herein, such as the device 100) to remove the PCB 7700 from the device 200. In some implementations, the PCB 7700 is a flexible PCB and includes an electrode pad or electrode array 7701 comprising one or more electrodes at a distal end of the PCB 7700, the electrode pad or array 7701 configured to split apart responsive to a sufficient force being applied to it. In some implementations, the PCB 7700 and/or the electrode pad/array 7701 has a relief cut through it so that it splits apart at the relief cut responsive to a sufficient force being applied to the PCB 7700 and/or electrode pad/array 7701.

In some implementations, the PCB 7700 includes a pair of leads 7702 that each extend proximally from a respective portion or half of the electrode pad/array 7701. In some implementations, the PCB 7700 can also include a reference electrode 7704 for each lead 7702. In some implementations, the leads 7702 are configured to extend from the electrode pad/array 7701 to a proximal end of the delivery system 202.

In some implementations, there is a change in direction in each lead 7702 that is configured to cause the PCB 7700 and/or the electrode pad/array 7701 to split apart responsive to a pulling force on the leads 7702. In some implementations, once split apart, each portion or half of the electrode pad/array 7701 exits its respective side of the clasp 230 by going around the friction enhancing elements or barbs 236, using the friction enhancing elements or barbs 236 as a fulcrum. In some implementations, the PCB 7700 is secured to the clasp 230, e.g., to the frame of the clasp 230 or to an optional cover 240 covering the clasp 230. When installed on the device 200, the leads 7702 extend around the friction enhancing elements or barbs 236 of the tissue engagement portion or clasp 230. Pulling on a proximal end of the leads 7702 causes the electrode pad/array 7701 to split apart and to pass around the friction enhancing elements or barbs 236 before entering a lumen of the delivery system 202 to be removed from the patient as part of the process of implanting the device 200. The PCB 7700 can be configured to exit the clasp 230 at or near a first arm 234 (e.g., a moveable arm), at or near a second arm 232 (e.g., a fixed arm, a paddle, etc.), at or near the joint portion 238, and/or at or near friction enhancing elements or barbs 236.

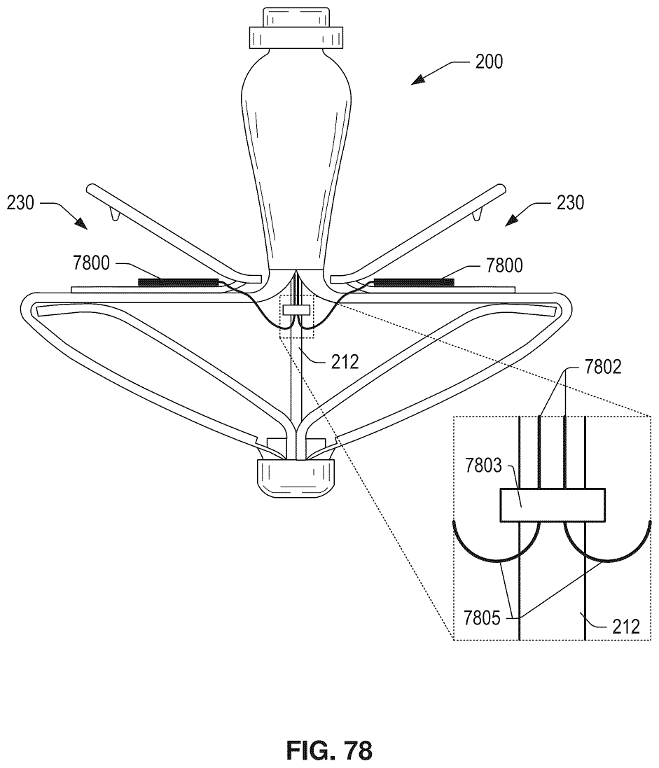

FIG. 78 illustrates an electrode 7800 that is removable from a device 200. The electrode 7800 is coupled to wires 7805 extending from the electrode 7800 towards the actuation element 212. The electrode 7800 comprises a flexible PCB or a flexible electrode releasably secured to the tissue engagement portion or clasp 230. The wires 7805 extend from the electrode 7800 to a collar 7803. In some implementations, the collar 7803 is coupled to the actuation element 212 so that the collar 7803 rotates in response to rotation of the actuation element 212. In some implementations, the collar 7803 is configured to secure the wires 7805 and to secure electrical leads 7802 that extend to the proximal end of a delivery system (such as the delivery systems 102, 202), the electrical leads 7802 electrically coupled to the wires 7805.

In some implementations, the electrode 7800 is configured to be removed responsive to rotation of the actuation element 212 during implant disconnection. Rotation of the actuation element 212 causes the collar 7803 to rotate which pulls the wires 7805 coupled to the electrode 7800 which in turn pulls the electrode 7800 off of the clasp 230. Continued rotation of the actuation element 212 causes the electrodes 7800 and wires 7805 to wrap around the actuation element 212, in preparation for removal.

In some implementations, after the device 200 is closed, rotation of the actuation element 212 pulls the electrodes 7800 out of the clasps 230 without removing the leaflets and wraps the electrodes 7800 and wires 7805 around the actuation element 212. That is, in some implementations, the actuation element 212 acts as a spool wrapping the excess wire and electrodes up around it, gradually and gently pulling them out from the device 200. The whole wrap is then pulled out from the patient, leaving no electrode or wire in the patient. The wires 7805 and electrical leads 7802 can be secured to the collar 7803 using an adhesive. In some implementations, the wires 7805 and electrical leads 7802 are directly affixed to the actuation mechanism 212 (e.g., using an adhesive) without the use of the collar 7803.

Advantageously, this configuration is transparent to the user because the release of the device remains unchanged. That is, there are no additional steps to remove the electrode 7800 from the device 200 because closing the device 200 with the actuation element 212 is sufficient to remove the electrode 7800 and wires 7805. The force multiplier of the knob of the delivery system 202 to unscrew the actuation element 212 is already built in so no added knob or other such mechanism is required to remove the electrodes 7800. This means there is no added complexity for the user or during manufacturing and assembly from that perspective. Furthermore, if the force required to pull the electrodes 7800 out of the device 200 is somewhat large, the user will not feel it because the knob would apply a gradual, controlled force.

Detaching Electrical Connection from Impedance Measurement Sensors

In some implementations, the electrodes are not removed from the device after implanting the device. However, it is still necessary to provide an electrical path from the electrodes to the proximal end of the delivery device to provide bioimpedance-based feedback to the user (e.g., regarding leaflet capture). Accordingly, disclosed herein are methods and devices for releasably coupling electrical leads to an electrode coupled to a device, such as the devices 100, 200, 300, 8200, 8810, 40256, etc. described herein.

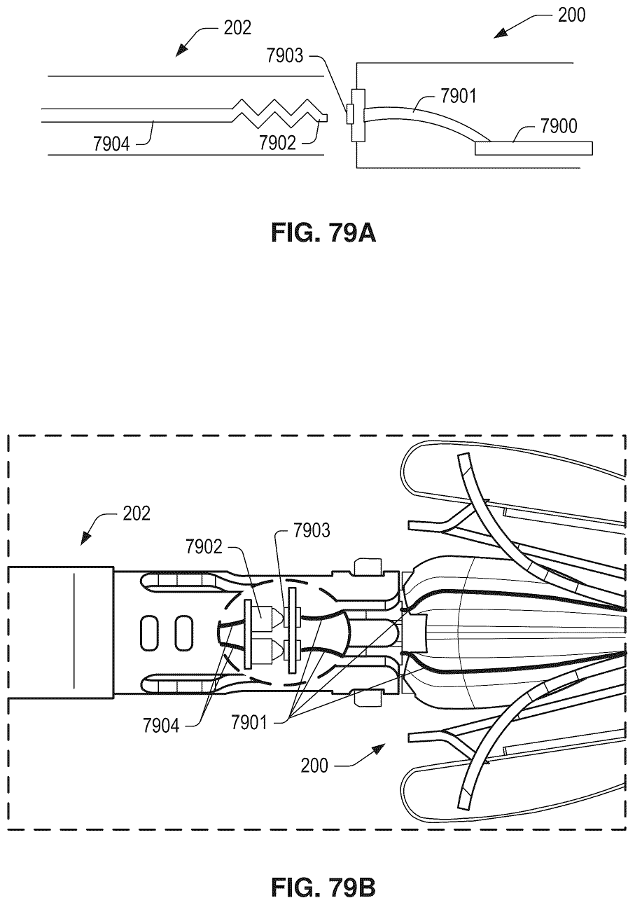

FIGS. 79A and 79B illustrate example spring pin electrical connectors 7902 configured to extend to a distal end of the delivery system 202 to provide electrical connection with wires 7901 of an electrode 7900 coupled to the device 200. The spring pin electrical connectors 7902 are configured to contact an electrical pad 7903 that is electrically coupled to the wires 7901 of the electrode 7900 when the device 200 is coupled to the delivery system 202 during implanting. Upon removing the device 200 from the delivery system 202, the spring pin electrical connectors 7902 move away from the electrical pad 7903, severing the electrical connection with the electrode 7900. The delivery device 202 is then removed along with the spring pin electrical connectors 7902 and associated wires 7904.

The spring pin electrical connectors 7902 are configured to use spring forces parallel to the shaft of the delivery system 202 to provide good electrical contact between wires 7901 (and electrical pad 7903) and the spring pin electrical connectors 7902. The spring pin electrical connectors 7902 are also easily removed as there is no physical coupling that secures the spring pin electrical connectors 7902 to the device 200 or to the wires 7901. The spring force of the spring pin electrical connectors 7902 is configured to assist in detaching the spring pin electrical connectors 7902 from the device 200, making sure there is a complete release of the device 200 from the delivery system 202. In some implementations, the spring pin electrical connectors 7902 are part of the device, replacing the electrical pad 7903. In such implementations, the distal end of the delivery system 202 includes electrical pads to interface with the spring pin electrical connectors of the device 200, essentially reversing the roles illustrated in FIGS. 79A and 79B.

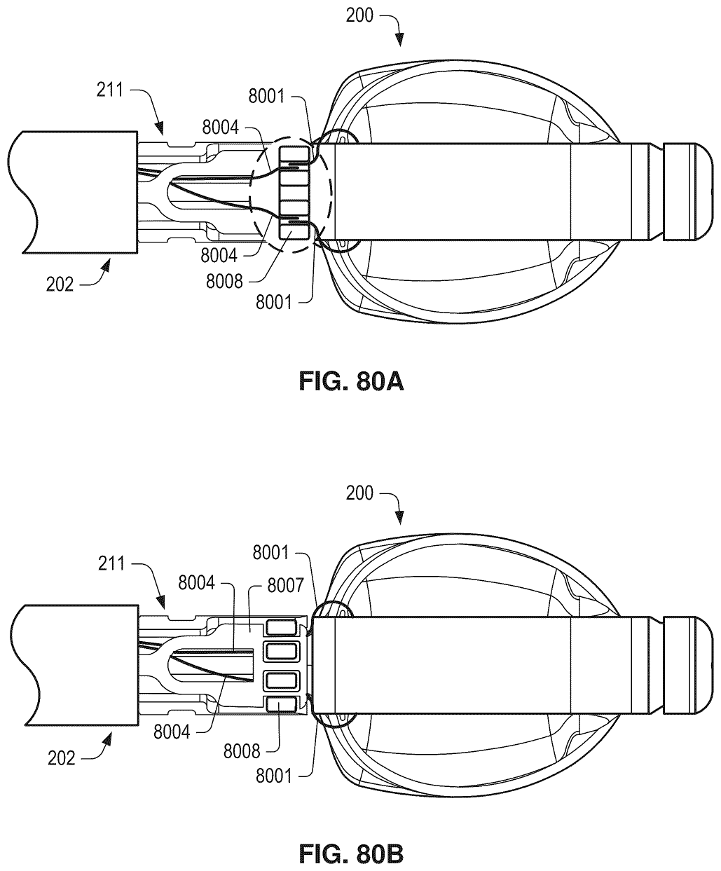

FIGS. 80A and 80B illustrate using radial forces via fingers 8007 of the delivery system 202 to couple the wires 8004 coming from the delivery system 202 to electrical leads 8001 coupled to an electrode (not shown) of the device 200. FIG. 80A shows the fingers 8007 as transparent to show the electrical connection between the wires 8004 to the electrical leads 8001, whereas FIG. 80B shows the fingers 8007 as opaque. The delivery system 202 includes a proximal component or collar 211 with grooves 8008 that are configured to mate with the fingers 8007 of the delivery system 202. In some implementations, the wires 8004 are coupled to the fingers 8007 and the electrical leads 8001 are secured in individual grooves 8008 such that when the fingers 8007 mate with the grooves 8008, the wires 8004 contact the electrical leads 8001 to form an electrical connection between the wires 8004 (that extend from the device 200 to the proximal end of the delivery system 202) and the electrical leads 8001 (that extend from an electrode or PCB coupled to the clasp 230 of the device 200 to the proximal collar 211). In some implementations, both the inner side of the fingers 8007 and the grooves 8008 are coated with an insulative material to electrically isolate each measurement channel.

Advantageously, this method for electrically coupling the wires 8004 with the electrical leads 8001 is transparent to the user. The normal implant release procedure is not changed. Additionally, the electrical connection is maintained until the implant is released enabling leaflet indication until the end of the implant procedure.

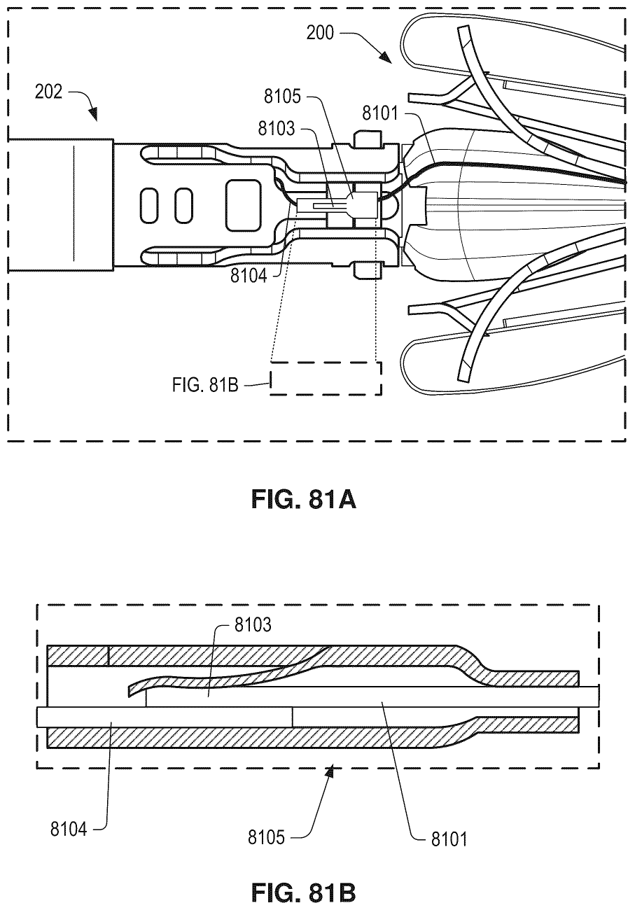

FIGS. 81A and 81B illustrate the use of a tube 8105 to enable releasable electrical contact between wires 8104 and electrical leads 8101. The tube 8105 can be secured at a distal end of the delivery system 202 using any suitable mechanism, such as a frame that holds the tube 8105 in a targeted location, the frame being a U-shaped frame that friction fits at the attachment portion 205 of the device 200 (e.g., near the proximal component or collar 211). The frame can include holes for the actuation element 212 and fingers of the delivery system 202. The frame can be made of a polymer to electrically isolate the wires 8104 from each other and to provide electrical isolation for each measurement channel (e.g., each connection between a wire 8104 and an electrical lead 8101). The frame can include a tube 8105 for each measurement channel.

When attaching the device 200 to the delivery system 202, the electrical leads 8101 can be inserted into the tubes 8105 along with the wires 8104, an electrical lead and a wire forming a measurement channel in a respective tube 8105. In some implementations, the tube 8105 can include a leaf spring 8103 to provide a clamping force on the wire 8104 and electrical lead 8101 to ensure a good electrical connection. Removal of the device 200 from the delivery system 202 causes the electrical leads 8101 to pull out from the tubes 8105.

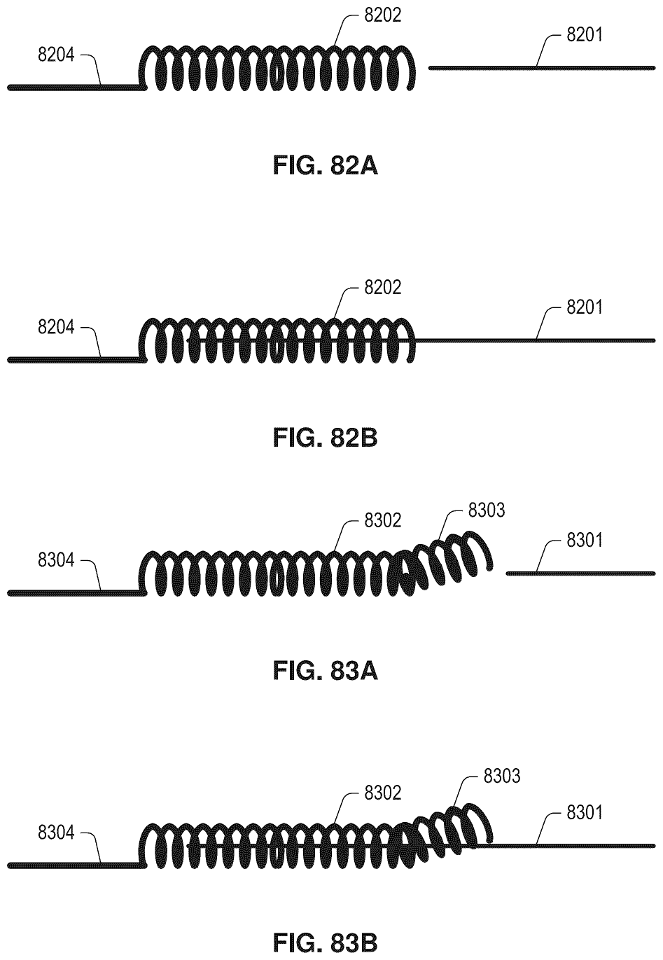

FIGS. 82A and 82B illustrate a coil crimp 8202 configured to provide releasable electrical contact between wires 8204 and electrical leads 8201. The wires 8204 are configured to run from a proximal end of the delivery system 202 to a distal end to electrically couple with the electrical leads 8201, the electrical leads 8201 electrically coupled to an electrode as described herein. The wires 8204 terminate with a coil crimp 8202 that is configured to couple with the electrical leads 8201 due to a friction fit.

The coil crimp 8202 can be formed from a distal portion of the wires 8204. The coil crimp 8202 can be wrapped with different pitches and different diameters to create more or less holding force on the inserted electrical lead 8201. In some implementations, the coil crimp 8202 can be made with a shape memory alloy, such as nitinol, in a martensite state (e.g., at 37 deg. C.) so that it switches to austenite when heated and expands slightly. The expansion is configured to release the electrical lead 8201. Expansion can be triggered by applying a targeted current through the wire 8204 to release the electrical lead 8201.

In some implementations, the coil crimp 8202 can be formed by wrapping the distal end of the wire 8204 around a mandrel that is slightly smaller than the electrical lead 8201 to achieve a friction fit with the electrical lead 8201. The friction fit is configured to maintain the electrical connection between the wire 8204 and the electrical lead 8201 until the coil crimp 8202 is caused to expand to release the electrical lead 8201. Once expanded, the wire 8204 can be withdrawn into the delivery system 202 to terminate the electrical and physical coupling of the coil crimp 8202 with the electrical lead 8201.

FIGS. 83A and 83B illustrate a coil connection socket 8302 configured to provide releasable electrical contact between wires 8304 and electrical leads 8301. The wires 8304 are configured to run from a proximal end of the delivery system 202 to a distal end to electrically couple with the electrical leads 8301, the electrical leads 8301 electrically coupled to an electrode as described herein. In some implementations, the wires 8304 terminate with a coil connection socket 8302 that is configured to couple with the electrical leads 8301 due to a friction fit, similar to the coil crimp 8202 described herein with reference to FIGS. 82A and 82B. A difference with the coil connection socket 8302 is that a distal portion 8303 of the coil is bent up so that the electrical lead 8301 is inserted at the bent location of the distal portion 8303 to increase the friction force on the electrical lead 8301 to increase the strength of the connection between the wire 8304 and the electrical lead 8301. For example, the spring constant of the coil determines the force applied to the electrical lead 8301 when a few coils are bent out of the way and the electrical lead 8301 is inserted and the bent part of distal portion 8303 of the coils are released and snap back in place.

The coil connection socket 8302 can be formed from a distal portion of the wires 8304. The coil connection socket 8302 can be wrapped with different pitches and different diameters to create more or less holding force on the inserted electrical lead 8301. In some implementations, the coil connection socket 8302 can be made with a shape memory alloy, such as nitinol, in a martensite state (e.g., at 37 deg. C.) so that it switches to austenite when heated and expands slightly. The expansion is configured to release the electrical lead 8301. Expansion can be triggered by applying a targeted current through the wire 8304 to release the electrical lead 8301.

The coil connection socket 8302 can be formed by wrapping the distal end of the wire 8304 around a mandrel that is slightly smaller than the electrical lead 8301 to achieve a friction fit with the electrical lead 8301. The distal portion 8303 of the coil can be bent up to increase the friction fit. The friction fit is configured to maintain the electrical connection between the wire 8304 and the electrical lead 8301 until the coil connection socket 8302 is caused to expand to release the electrical lead 8301. Once expanded, the wire 8304 can be withdrawn into the delivery system 202 to terminate the electrical and physical coupling of the coil connection socket 8302 with the electrical lead 8301.

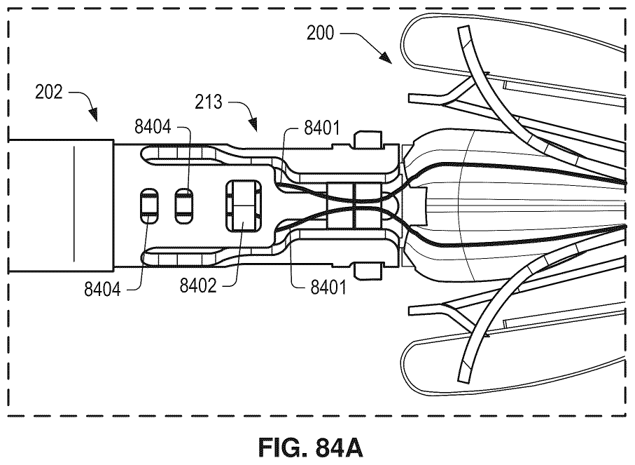

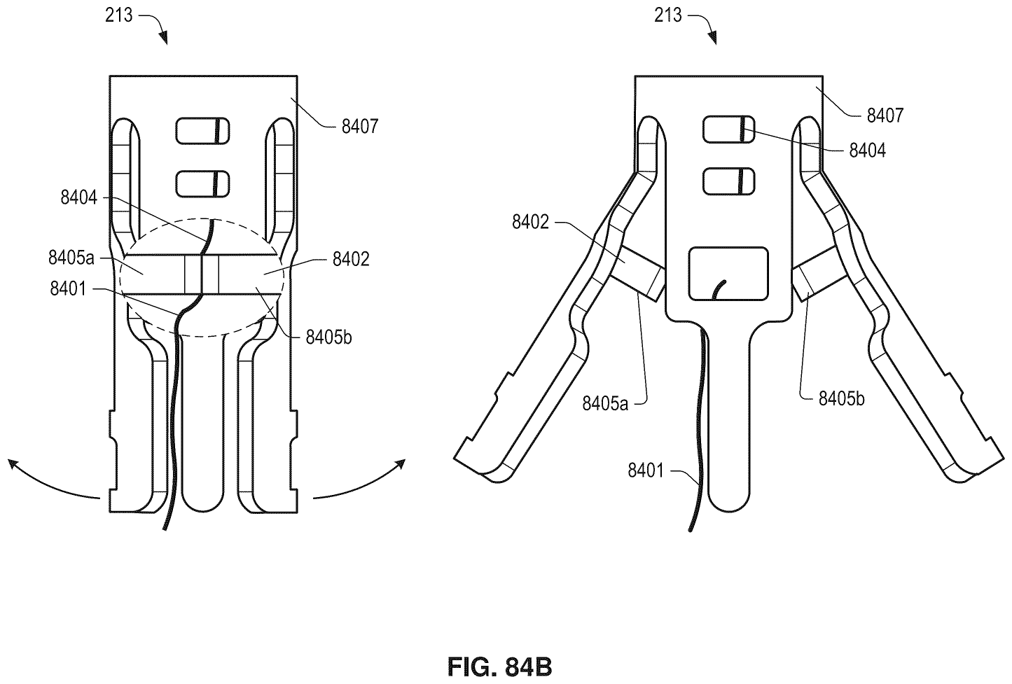

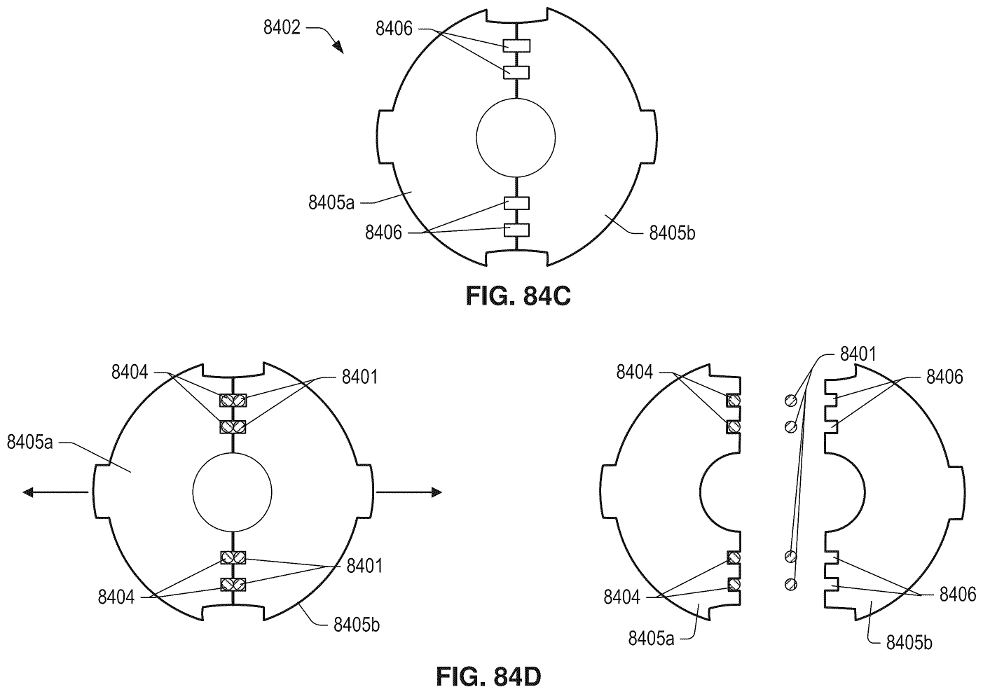

FIGS. 84A, 84B, 84C, and 84D illustrate an example disc crimp 8402 configured to provide releasable electrical connection between wires 8404 and electrical leads 8401. The wires 8404 are configured to run from a proximal end of the delivery system 202 to a distal end to electrically couple with the electrical leads 8401, the electrical leads 8401 electrically coupled to an electrode as described herein. In some implementations, the disc crimp 8402 provides slots 8406 which enable a wire 8404 to electrically couple to an electrical lead 8401 through physical contact. The slots 8406 are sized such that the wire and the electrical lead 8401 are pushed together in the slot 8406 to maintain physical contact with one another.

In some implementations, the disc crimp 8402 comprises disc halves 8405a, 8405b. While the fingers 8407 of the capture mechanism 213 are engaged with the device 200, the fingers 8407 hold the disc halves 8405a, 8405b together so the disc crimp 8402 can hold the wires 8404 and the electrical leads 8401 together. In some implementations, each disc half 8405a, 8405b is secured to a corresponding finger 8407 of the capture mechanism 213. When the fingers 8407 are closed, the slots are configured to electrically connect wires 8404 and electrical leads 8401. When the fingers 8407 open to release the device 200, the disc halves 8405a, 8405b separate, thereby allowing the wires 8404 to disconnect from the electrical leads 8401.

In some implementations, the disc halves 8405a, 8405b can be made of a polymer to be electrically insulative, as described herein. The disc halves 8405a, 8405b can be secured to the fingers 8407 using any suitable means, such as adhesives. In some implementations, windows can be cut into the fingers 8407 and portions of the disc halves 8405a, 8405b can be inserted through the windows to form a friction fit or the portions of the disc halves 8405a, 8405b can be otherwise affixed to the fingers 8407 using the windows, such as by melting into the window to create a rivet or snap-like connection. In some implementations, the disc halves 8405a, 8405b can be a shape set alloy and welded onto the fingers 8407 with electrically insulative coating on the inside holes where the wires 8404 and electrical leads 8401 are crimped. The force of the fingers 8407 locked around the device 200 to provide the crimp force between the disc halves 8405a, 8405b to close the connection between the wires 8404 and the electrical leads 8401.

Advantageously, the disc crimp 8402 makes the disconnection of the wires 8404 and the electrical leads 8401 transparent to the user because there is no additional action to be taken to release the electrical connection. There is also little or minimal risk of accidentally or prematurely releasing the wires to terminate the electrical connection before the device 200 is ready because the electrical connection is maintained until the device 200 is released.

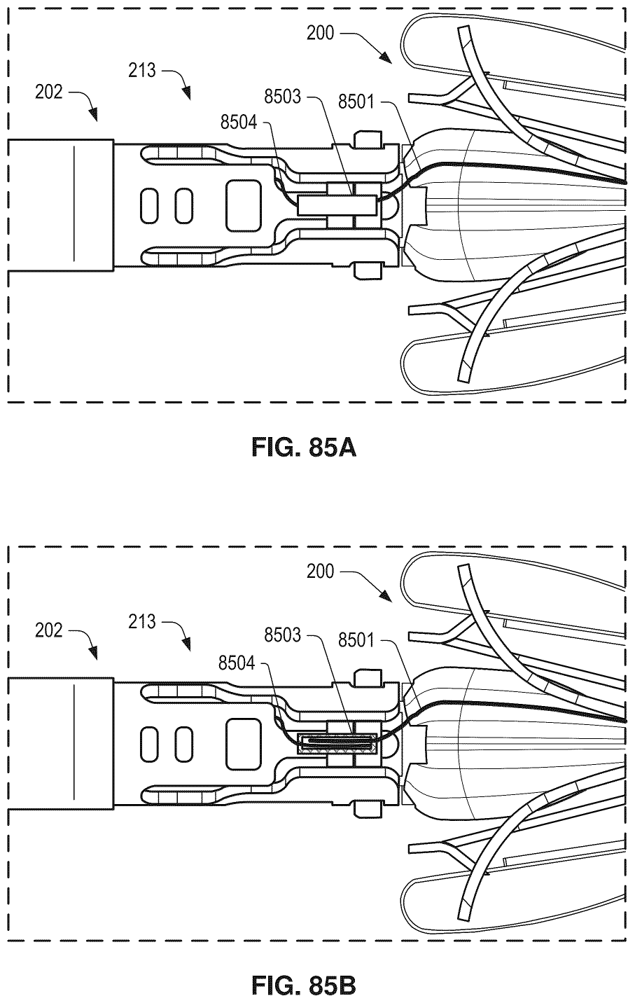

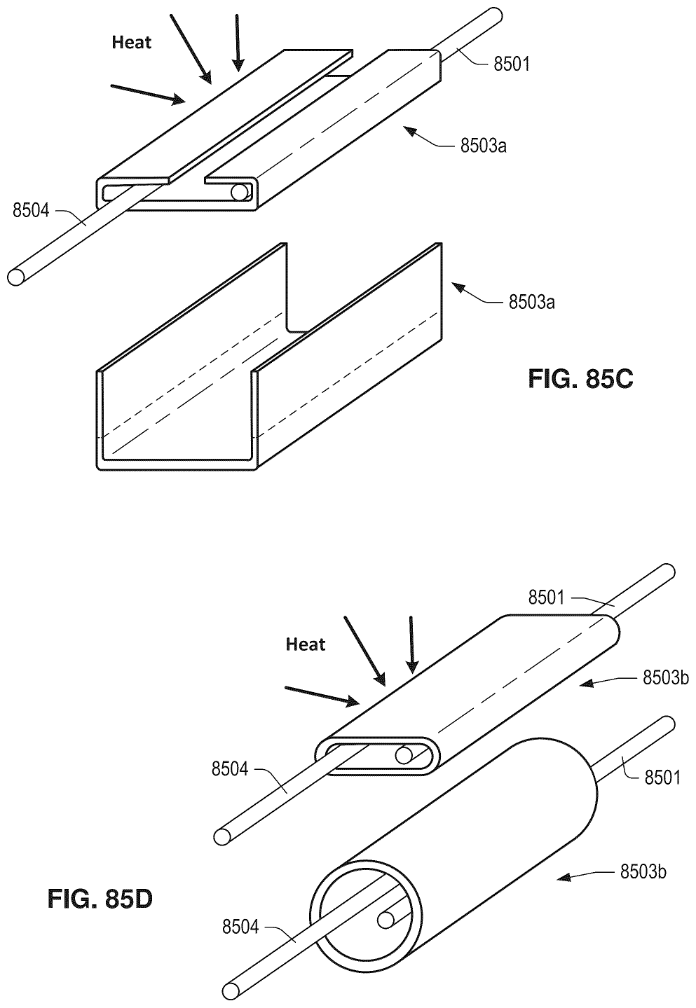

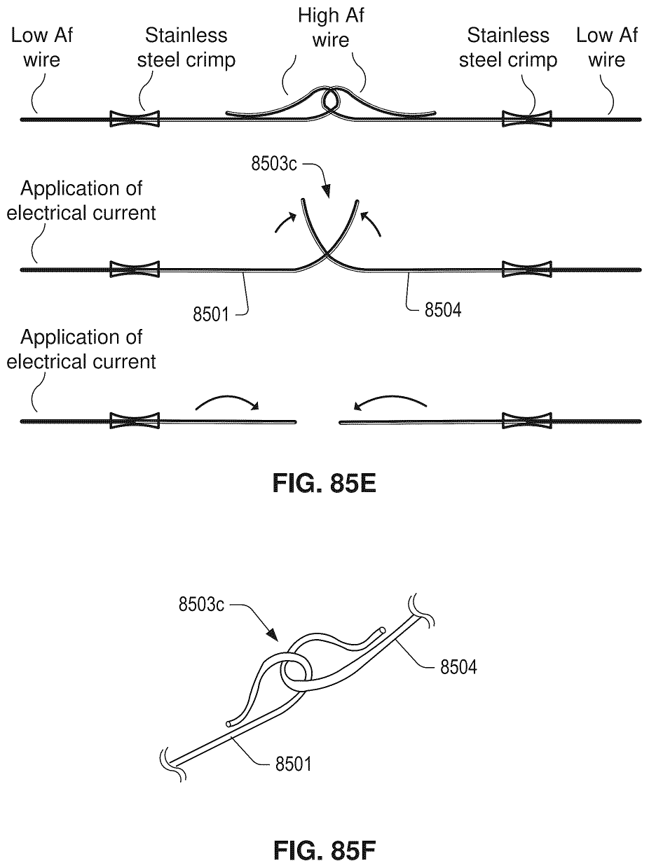

FIGS. 85A, 85B, 85C, 85D, 85E, and 85F illustrate examples of heat-activated electrical connectors 8503 to provide releasable electrical connections between wires 8504 and electrical leads 8501. The wires 8504 are configured to run from a proximal end of the delivery system 202 to a distal end to electrically couple with the electrical leads 8501, the electrical leads 8501 electrically coupled to an electrode as described herein. In some implementations, the heat-activated electrical connectors 8503 are configured to change shape with the application of heat or current to change shape. In some implementations, the change in shape releases the wires 8504 and the electrical leads 8501 to disconnect the electrical connection. In some implementations, heating the heat-activated electrical connectors 8503 transiently causes the connector to open to release the electrical leads 8501 to enable release of the device 200 from the delivery system 202 via the capture mechanism 213.

The heat-activated electrical connectors 8503 can be made of a shape set alloy, such as Nitinol, that is heat set to open upon being heated above its transition temperature. The transition temperature can be configured to be higher than body temperature (e.g., about 50° C.). Until heating, the heat-activated electrical connectors 8503 serve as a crimp that connects the wire 8504 of the delivery system 202 to the electrical lead 8501 of the device 200 (e.g., the heat-activated electrical connector 8503 is wrapped around both).

In some implementations, when it is desired to release the wire 8504 and the electrical lead 8501, (e.g., just before mechanically releasing the device 200), the heat-activated electrical connector 8503 is heated, causing it to transiently open. The heat-activated electrical connectors 8503 can be fixedly attached to the device 200 or to the delivery system 202, so as to not embolize. Heating can be achieved by introducing heated saline or by applying a current via the wire of the delivery system 202 (e.g., the wire via which electrical signals were previously being received).

FIG. 85C illustrates an example heat-activated electrical connector 8503a that comprises a Nitinol laser-cut tube or sheet that is shape-set to have an open orifice. The Nitinol is heat-treated following the shape-setting procedure to have a 50C Af transition temperature, meaning the part is in a soft martensitic phase in room and body temperatures. The wire 8504 and the electrical lead 8501 are inserted into the heat-activated electrical connector 8503a and crimped inside it. Crimping is possible because the heat-activated electrical connector 8503a is in its soft martensitic phase. When the release of the wire 8504 and the electrical lead 8501 is desired, the heat-activated electrical connector 8503a is heated by saline or an electrical current. As a result of the heating the part momentarily transitions to austenite and recovers to its original shape with an open orifice. Following the recovery of the heat-activated electrical connector 8503a the wire 8504 and the electrical lead 8501 are free to be removed.

FIG. 85D illustrates an example heat-activated electrical connector 8503b that is similar to the heat-activated electrical connector 8503a except that the shape is flat in its martensitic phase and a cylinder in its austenite phase. The open crimp of the cylinder allows the wire 8504 and the electrical lead 8501 to be removed.

FIGS. 85E and 85F illustrate an example heat-activated electrical connector 8503c that is configured to shape-set ends of the wire 8504 and the electrical lead 8501 so that they hook one another. The heat-activated electrical connector 8503c comprises a wire assembly that has two sections that are crimped to each other by a stainless-steel laser cut crimp. The proximal wire section of the electrical lead 8501 can be a low Af wire, e.g., 10C meaning that it is flexible at body temperature, and the distal wire section of the electrical lead 8501 can be a high Af wire, e.g., 50C meaning that it is soft at body temperature. Similarly, the proximal wire section of the wire 8504 can be a low Af wire, e.g., 10C meaning that it is flexible at body temperature, and the distal wire section of the wire 8504 can be a high Af wire, e.g., 50C meaning that it is soft at body temperature. The soft section is shape set in a straight geometry. The soft sections are looped around each other to form a connection feature. To release the wires, electrical current is introduced and heats the distal section for a split second. As a result of the heat the wire loops straighten, and the wires can be removed or disconnected. In some implementations, hot saline can be used to straighten the wires.

### Example Systems for Bioimpedance-Based Feedback

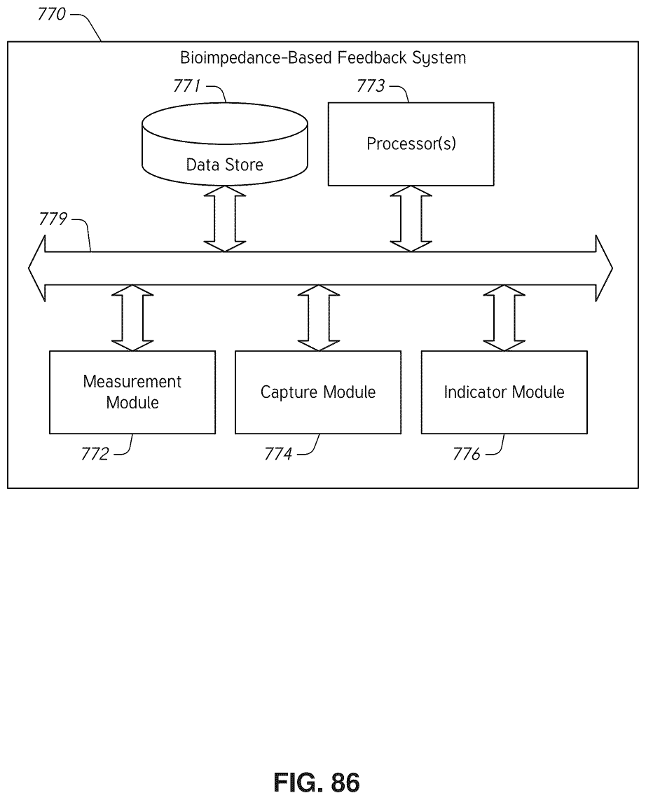

FIG. 86 illustrates a block diagram of an example system 770 (e.g., a bioimpedance-based feedback system, bioimpedance system, feedback system, etc.) configured to measure bioimpedance signals, determine tissue status (e.g., capture status, insertion status, etc.) with respect to a system, device, apparatus, etc. (e.g., any of the systems, devices, apparatuses, etc. disclosed herein), and/or to display or otherwise provide indicators associated with the determined status. The system 770 can employ any process, procedure, algorithm, or method described herein for measuring bioimpedance and determining tissue status with respect to an implant.

The system 770 (e.g., a bioimpedance-based feedback system, bioimpedance system, feedback system, etc.) can include hardware, software, and/or firmware components for bioimpedance-based feedback. The system 770 includes a data store 771, one or more processors 773, a measurement module 772, a capture module 774 (while referred to here as a “capture module”, this module can also be referred to as a “status module” and provide status indications other than regarding capture, e.g., insertion status, contact status, etc.), and an indicator module 776. Components of the system 770 can communicate with one another, with external systems, and/or with other components of a network using communication bus 779. The system 770 can be implemented using one or more computing devices. For example, the system 770 can be implemented using a single computing device, multiple computing devices, a distributed computing environment, or it can be located in a virtual device residing in a public or private computing cloud. In a distributed computing environment, one or more computing devices can be configured to provide the measurement module 772, the capture module 774, and the indicator module 776 to provide the described functionality.

In some implementations, the system 770 includes the measurement module 772 to acquire or receive electrical signals from electrical components (e.g., sensors, electrodes, arrays, PCBs, etc., such as the electrical sensor 6864 described herein with reference to FIG. 68.). In some implementations, the electrical signals correspond to bioimpedance signals and can also correspond to resistance, capacitance, voltage, current, components of impedance, and the like. The measurement module 772 is also configured to determine an impedance value based on the acquired bioimpedance signals. Thus, the measurement module 772 is configured to interface with hardware components that generate electrical signals and the measurement module 772 can implement one or more algorithms to determine electrical measurements, such as a bioimpedance measurement, based on the electrical signals from the hardware components.

In some implementations, the system 770 includes the capture module 774 to determine a capture status based on the bioimpedance measurements by the measurement module 772. The bioimpedance measurements (as well as resistance, inductance, capacitance, voltage, and/or current readings) measured by the measurement module 772 can be different based on the anatomy or anatomies that indicator electrodes are near or in contact with. Thus, the electrical characteristics measured by the measurement module 772, in particular the bioimpedance signals, can be used to determine the relative locations of a clasp, anchor, other device components, etc. and anatomy (e.g., tissue, etc.) that a device associated with the system 770 is in contact with, as described herein. For example, the value of the bioimpedance signal and/or changes in bioimpedance can signal that the electrodes are in blood, contacting tissue (e.g., leaflets), differentiating tissue (e.g., leaflet tissue versus chord tissue), transitioning from being primarily in contact with blood to being partially or primarily in contact with tissue, and/or transitioning from being partially or primarily in contact with tissue to being primarily in contact with blood. Examples of signals and what they indicate are described herein with reference to FIGS. 54-58, 61A, 61B, 62C, 67A, and 67B. Thus, algorithms can be implemented by the capture module 774 to determine a capture status, or other similar status such as anchor deployment status, based on the measurements acquired by the measurement module 772.

In some implementations, the system 770 includes the indicator module 776 to indicate results from the capture module 774. An example indicator is described herein with reference to FIG. 62D. However, other indicators may be employed such as one or more displays, LEDs, alarms, speakers, etc. and the indicator module 776 can interface with one or more of these indicators to relay information from the capture module 774 and/or the measurement module 772. Thus, algorithms can be implemented by the indicator module 776 to convert output from the capture module 774 into an indicator for a user (e.g., using visual and/or audible indicators) or for another device or computer system (e.g., using analog or digital communication protocols).

In some implementations, the system 770 includes one or more processors 773 that are configured to control operation of the measurement module 772, the capture module 774, the indicator module 776, and the data store 771. The one or more processors 773 implement and utilize the software modules, hardware components, and/or firmware elements configured to provide bioimpedance-based feedback. The one or more processors 773 can include any suitable computer processors, application-specific integrated circuits (ASICs), field programmable gate array (FPGAs), or other suitable microprocessors. The one or more processors 773 can include other computing components configured to interface with the various modules and data stores of the system 770.

In some implementations, the system 770 includes the data store 771 configured to store configuration data, measurement data, analysis parameters, control commands, databases, algorithms, executable instructions (e.g., instructions for the one or more processors 773), and the like. The data store 771 can include a combination of memory and/or storage devices. The data store 771 can be any suitable data storage device or combination of devices that include, for example and without limitation, random access memory, read-only memory, solid-state disks, hard drives, flash drives, and the like. For example, the data store 771 can include any suitable non-transitory computer readable medium. In some implementations, one or multiple or all of various steps, methods, procedures, algorithms, etc. of the systems, apparatuses, and/or devices herein can be stored on a non-transitory computer readable medium. In some implementations, the data store 771 can be configured to store computer executable instructions to cause the one or more processors 773 to perform any of the algorithms, procedures, processes, or methods described herein. Similarly, the measurement module 772, the capture module 774, and the indicator module 776 can represent hardware or software modules that provide the described functionality in conjunction with the data store 771 and the one or more processors 773.

### ADDITIONAL EXAMPLES

The following includes additional description of examples. The examples are intended to illustrate examples of combinations of elements and are not intended to limit the scope of any particular example or implementation disclosed herein.

Example 1: A device comprising: a tissue engagement portion comprising a first surface and a second surface, the tissue engagement portion configured such that the first surface and the second surface can close or be moved closer together to engage and/or capture tissue in the tissue engagement portion, at least one of the first surface and the second surface being movable to form a capture region between the first surface and the second surface for capturing the tissue; and/or two or more electrodes coupled to the tissue engagement portion, wherein the device is configured such that: an electrical signal can be applied to the two or more electrodes, and/or a bioimpedance signal can be measured responsive to the electrical signal applied, the bioimpedance signal providing an indication of a status of the tissue relative to the tissue engagement portion.

Example 2: The device of example 1, wherein the status includes under insertion of tissue in the tissue engagement portion.

Example 3: The device of any of examples 1-2, wherein the status includes full insertion of tissue in the tissue engagement portion.

Example 4: The device of any of examples 1-3, wherein the status includes over insertion of tissue in the tissue engagement portion.

Example 5: The device of any of examples 1-4, wherein the status includes angled insertion of tissue in the tissue engagement portion.

Example 6: The device of any of examples 1-5, wherein the status includes insertion of non-targeted tissue in the tissue engagement portion.

Example 7: The device of example 6, wherein the non-targeted tissue includes chordae tendineae.

Example 8: The device of any of examples 1-7, wherein the status includes insertion of tissue in the tissue engagement portion while the tissue engagement portion is in an open configuration comprising the first surface and the second surface being apart from each other.

Example 9: The device of any of examples 1-8, wherein the indication of the status is configured to be used to generate a visual indicator for a user of the status.

Example 10: The device of example 9, wherein the visual indicator is configured to indicate one or more of no tissue insertion, under tissue insertion, full tissue insertion, and/or over tissue insertion.

Example 11: A system, an apparatus, and/or apparatus, the system, apparatus, and/or device comprising: an anchor comprising a first arm and a second arm (optionally “surface” can be used in place of “arm”, e.g., “first surface” in place of “first arm” and “second surface” in place of “second arm” in any of examples 11-27) configured such that the first arm and the second arm can close or be moved closer together to capture tissue in the anchor, at least one of the first arm and the second arm being movable to form a capture region therebetween for capturing the tissue; and/or two or more electrodes coupled to the anchor, wherein the system, apparatus, and/or device is configured such that: an electrical signal can be applied to the two or more electrodes, and/or a bioimpedance signal can be measured based on or in response to the applied electrical signal.

Example 12: The system, apparatus, and/or device of example 11, wherein the two or more electrodes comprise: a first electrode strip coupled to the first arm of the anchor near a first edge of the first arm; and/or a second electrode strip coupled to the first arm of the anchor near a second edge of the first arm, the second edge opposite the first edge, wherein the first electrode strip and the second electrode strip are parallel to each other and/or run along a length of the first arm.

Example 13: The system, apparatus, and/or device of example 12, wherein the first electrode strip and the second electrode strip are offset a prescribed distance from a free edge of the first arm of the anchor.

Example 14: The system, apparatus, and/or device of example 13, wherein the prescribed distance is at least 6 mm.

Example 15: The system, apparatus, and/or device of any of examples 12-14, wherein a first bioimpedance signal can be measured based on an applied electrical signal to the first electrode strip and/or a second bioimpedance signal can be measured based on an applied electrical signal to the second electrode strip.

Example 16: The system, apparatus, and/or device of example 15, wherein the first bioimpedance signal and the second bioimpedance signal indicate a status of the tissue between the first arm and the second arm of the anchor.

Example 17: The system, apparatus, and/or device of example 16, wherein a difference between the status indicated by the first bioimpedance signal and indicated by the second bioimpedance signal indicates an angled insertion of the tissue between the first arm and the second arm of the anchor.

Example 18: The system, apparatus, and/or device of example 16, wherein an average of the first bioimpedance signal and the second bioimpedance signal is used to determine the status of the tissue.

Example 19: The system, apparatus, and/or device of example 16, wherein the first bioimpedance signal and the second bioimpedance signal provide a continuous indication of tissue insertion between the first arm and the second arm.

Example 20: The system, apparatus, and/or device of example 19, wherein the continuous indication of the status is divided into quantized signal regions indicating four categories of status that include no insertion of the tissue, under insertion of the tissue, full insertion of the tissue, and/or over insertion of the tissue.

Example 21: The system, apparatus, and/or device of any of examples 12-20 further comprising a reference electrode configured to enable bipolar measurements of the bioimpedance signal.

Example 22: The system, apparatus, and/or device of example 21, wherein the bioimpedance signal can be measured in at least three configurations comprising the first electrode strip versus the reference electrode, the second electrode strip versus the reference node, and/or the first electrode strip versus the second electrode strip.

Example 23: The system, apparatus, and/or device of example 11, wherein the two or more electrodes comprise: a first electrode coupled to the first arm of the anchor near a free edge of the first arm, the free edge opposite a hinged edge coupled to a hinged edge of the second arm; and a second electrode coupled to the second arm of the anchor near a free edge of the second arm, the free edge opposite the hinged edge, wherein the first electrode and the second electrode are configured to contact one another with the anchor closed.

Example 24: The system, apparatus, and/or device of example 23, wherein bioimpedance signals measured with the first electrode and with the second electrode are configured to be used to determine a thickness of the tissue inserted into the anchor.

Example 25: The system, apparatus, and/or device of example 24, wherein the bioimpedance signals measured with the first electrode and with the second electrode are configured to be used to determine variation in thickness of the tissue as the tissue is inserted into the anchor.

Example 26: The system, apparatus, and/or device of example 25, wherein a cross-sectional map of the thickness of the tissue is generated based on the determined thickness and variation in thickness of the tissue.

Example 27: The system, apparatus, and/or device of any of examples 23-26, wherein bioimpedance signals are measured with the first electrode and with the second electrode while the anchor is partially closed to approximate the first electrode and the second electrode to the tissue inserted into the anchor.

Example 28: A system, an apparatus, and/or a device usable for repairing or treating a native valve and/or other tissue, the system, apparatus, and/or device comprising: a coaptation portion; an anchor portion coupled to the coaptation portion, the anchor portion comprising a tissue engagement portion, anchor, or clasp configured to capture tissue (e.g., a leaflet of the native valve) in the tissue engagement portion, anchor, or clasp; one or more flexible electrodes protruding away from the coaptation portion; and/or a reference electrode, wherein the system, apparatus, and/or device is configured such that: an electrical signal can be applied to the one or more flexible electrodes, and/or a bioimpedance signal can be measured based on or in response to the applied electrical signal to determine relative blood flow adjacent to the system, apparatus, and/or device.

Example 29: The system, apparatus, and/or device of example 28, wherein the one or more flexible electrodes are configured to measure blood flow through the native valve.

Example 30: The system, apparatus, and/or device of example 28, wherein the one or more flexible electrodes are configured to detect leakage through the native valve.

Example 31: The system, apparatus, and/or device of any of examples 28-30, wherein the one or more flexible electrodes are configured to deflect in response to blood flowing past the one or more flexible electrodes.

Example 32: The system, apparatus, and/or device of example 31, wherein the bioimpedance signal changes in response to deflection of the one or more flexible electrodes.

Example 33: The system, apparatus, and/or device of example 32, wherein the change in bioimpedance signal correlates to an amount of deflection which correlates to a blood flow rate through the valve.

Example 34: The system, apparatus, and/or device of any of examples 28-33, wherein the bioimpedance signal is configured to decrease in response to regurgitation blood volume through the native valve.

Example 35: The system, apparatus, and/or device of any of examples 28-34, wherein the reference electrode is coupled to an actuation element, the actuation element coupled to the coaptation portion and/or to the anchor portion.

Example 36: A system, an apparatus, and/or a device usable for repairing a native valve, the system, apparatus, and/or device comprising: a coaptation portion; an anchor portion coupled to the coaptation portion, the anchor portion comprising a tissue engagement portion, anchor, or clasp configured to capture tissue (e.g., a leaflet of the native valve) in the tissue engagement portion, anchor, or clasp; and/or one or more electrodes coupled to the anchor portion, wherein the system, apparatus, and/or device is configured such that: an electrical signal can be applied to the one or more electrodes, and/or a bioimpedance signal can be measured based on or in response to the applied electrical signal to determine forces on the system, apparatus, and/or device.

Example 37: The system, apparatus, and/or device of example 36, wherein the bioimpedance signal is correlated with a deflection of the coaptation portion or the anchor portion.

Example 38: The system, apparatus, and/or device of any of examples 36-37, wherein the deflection is correlated with a force applied to the system, apparatus, and/or device such that the bioimpedance signal is correlated with the force applied to the system, apparatus, and/or device.

Example 39: The system, apparatus, and/or device of any of examples 36-38, wherein the anchor portion further comprise an inner paddle coupled to an outer paddle that rotate relative to one another, wherein forces applied to the system, apparatus, and/or device change an opening distance between the inner paddle and the outer paddle.

Example 40: The system, apparatus, and/or device of any of examples 36-39, wherein a first electrode of the two or more electrodes is coupled to the inner paddle and a second electrode of the two or more electrodes is coupled to the outer paddle such that the change in the opening distance causes a change in the bioimpedance signal.

Example 41: A system, an apparatus, and/or a device usable for repairing a native valve, the system, apparatus, and/or device comprising: a tissue engagement portion, anchor, or clasp comprising a first arm and a second arm (optionally “surface” can be used in place of “arm”, e.g., “first surface” in place of “first arm” and “second surface” in place of “second arm” in any of examples 41-48) configured such that the first arm and the second arm can close or be moved closer together to capture tissue (e.g., a leaflet of the native valve) in the tissue engagement portion, anchor, or clasp, at least one of the first arm and the second arm being movable to form a capture region therebetween for capturing the tissue (e.g., a leaflet of the native valve); a plurality of electrodes coupled to the tissue engagement portion, anchor, or clasp, the plurality of electrodes electrically coupled in series using an electrical lead; and/or a plurality of electrical components electrically coupled in series with the plurality of electrodes using the electrical lead, wherein the system, apparatus, and/or device is configured such that: an electrical signal can be applied to the plurality of electrodes through the electrical lead, and/or a bioimpedance signal can be measured for the plurality of electrodes based on or in response to the applied electrical signal, and/or measured bioimpedance values are determined for each electrode of the plurality of electrodes based on electrical characteristics of the plurality of electrical components and the electrical signal.

Example 42: The system, apparatus, and/or device of example 41, wherein one or more electrical components of the plurality of electrical components is coupled in series between a pair of electrodes of the plurality of electrodes using the electrical lead.

Example 43: The system, apparatus, and/or device of example 42, wherein the one or more electrical components includes a resistor, capacitor, or inductor.

Example 44: The system, apparatus, and/or device of example 43, wherein the one or more electrical components in series between the pair of electrodes of the plurality of electrodes have different electrical characteristics than another of the one or more electrical components in series between a different pair of electrodes of the plurality of electrodes.

Example 45: The system, apparatus, and/or device of any of examples 41-44, wherein the electrical signal can be applied with a predetermined current and frequency.

Example 46: The system, apparatus, and/or device of any of examples 41-45, wherein the electrical lead comprises a single electrical lead.

Example 47: The system, apparatus, and/or device of any of examples 41-46, wherein: a resistor with a fixed resistance value is coupled in series between a first electrode and a second electrode of the plurality of electrodes; a capacitor with a fixed capacitance value is coupled in series between the second electrode and a third electrode of the plurality of electrodes; and/or an inductor with a fixed inductance value is coupled in series between the third electrode and a fourth electrode of the plurality of electrodes.

Example 48: The system, apparatus, and/or device of example 47, wherein the measured bioimpedance signals of the first, second, third, and fourth electrodes of the plurality of electrodes depends on the fixed resistance of the resistor, the fixed capacitance of the capacitor, and the fixed inductance of the inductor in conjunction with a predetermined frequency and current of the applied electrical signal.

Example 49: A system, an apparatus, and/or a device usable for repairing a native valve, the system, apparatus, and/or device comprising: a tissue engagement portion, anchor, or clasp comprising a first arm and a second arm (optionally “surface” can be used in place of “arm”, e.g., “first surface” in place of “first arm” and “second surface” in place of “second arm” in any of examples 49-50) configured such that the first arm and the second arm can close or be moved closer together to capture tissue (e.g., a leaflet of the native valve) in the tissue engagement portion, anchor, or clasp, at least one of the first arm and the second arm being movable to form a capture region therebetween for capturing the tissue (e.g., a leaflet of the native valve); a plurality of electrodes coupled to the tissue engagement portion, anchor, or clasp; and/or an analog-to-digital converter (ADC) chip coupled to the tissue engagement portion, anchor, or clasp and electrically coupled to the plurality of electrodes; and/or a single electrical lead configured to direct signals from the ADC chip to a measurement system, wherein the system, apparatus, and/or device is configured such that: an electrical signal can be applied to the plurality of electrodes through the ADC chip, the ADC chip digitizes a bioimpedance signal from each of the plurality of electrodes, the digitized bioimpedance signal is transmitted over the single electrical lead to the measurement system, and/or the bioimpedance signal is determined for each of the plurality of electrodes based on or in response to the applied electrical signal and/or the digitized bioimpedance signal.

Example 50: The system, apparatus, and/or device of example 46, wherein the digitized bioimpedance signal is sent over the single electrical lead using digital packets.

Example 51: A system, an apparatus, and/or a device usable for repairing a native valve, the system, apparatus, and/or device comprising: a tissue engagement portion, anchor, or clasp comprising a first arm and a second arm (optionally “surface” can be used in place of “arm”, e.g., “first surface” in place of “first arm” and “second surface” in place of “second arm” in any of examples 51-61) configured such that the first arm and the second arm can close or be moved closer together to capture tissue (e.g., a leaflet of the native valve) in the tissue engagement portion, anchor, or clasp, at least one of the first arm and the second arm being movable to form a capture region therebetween for capturing the tissue (e.g., a leaflet of the native valve); and/or a flexible printed circuit board (PCB) comprising a body, one or more electrodes coupled to the body, and/or an electrical lead extending away from the body, the flexible PCB coupled to the tissue engagement portion, anchor, or clasp using one or more sutures, wherein the system, apparatus, and/or device is configured such that: an electrical signal can be applied to the one or more electrodes through the electrical lead of the flexible PCB, and/or a bioimpedance signal can be measured using the electrical lead based on or in response to the applied electrical signal.

Example 52: The system, apparatus, and/or device of example 51 further comprising a cover that covers the tissue engagement portion, anchor, or clasp, wherein the flexible PCB is secured to the cover that covers the tissue engagement portion, anchor, or clasp to couple the flexible PCB to the tissue engagement portion, anchor, or clasp.

Example 53: The system, apparatus, and/or device of example 51 further comprising a cover that covers the tissue engagement portion, anchor, or clasp, wherein the flexible PCB is secured to the first arm of the tissue engagement portion, anchor, or clasp under the cover that covers the tissue engagement portion, anchor, or clasp to couple the flexible PCB to the tissue engagement portion, anchor, or clasp.

Example 54: The system, apparatus, and/or device of any of examples 51-53, wherein the flexible PCB comprises one or more physical features that facilitate removal of the flexible PCB from the tissue engagement portion, anchor, or clasp by applying a force to the electrical lead.

Example 55: The system, apparatus, and/or device of example 54, wherein: the one or more physical features comprise a stress concentration point comprising a narrow connection point between two openings, the one or more sutures is configured to extend through the two openings and over the narrow connection point to couple the flexible PCB to the tissue engagement portion, anchor, or clasp, and applying a force to the electrical lead causes the narrow connection point to break thereby releasing the flexible PCB from the tissue engagement portion, anchor, or clasp and leaving the one or more sutures coupled to the tissue engagement portion, anchor, or clasp.

Example 56: The system, apparatus, and/or device of example 54, wherein: the one or more physical features comprise a Y-shaped protrusion extending from an end of the body the flexible PCB opposite an end from which the electrical lead extends away from the body of the flexible PCB, the Y-shaped protrusion includes a pair of legs extending away from a bridge portion that extends away from the body of the flexible PCB, the bridge portion forming a rotation cutout configured to facilitate rotation of the pair of legs toward one another with an inward force applied to the pair of legs, the one or more sutures are configured to extend over the bridge portion to secure the flexible PCB to the tissue engagement portion, anchor, or clasp, and/or applying a force to the electrical lead causes the suture to push the pair of legs toward one another to allow the flexible PCB to slide from under the suture thereby releasing the flexible PCB from the tissue engagement portion, anchor, or clasp and leaving the one or more sutures coupled to the tissue engagement portion, anchor, or clasp.

Example 57: The system, apparatus, and/or device of example 54, wherein: the one or more physical features comprise a round protrusion extending from a side of the body the flexible PCB, the round protrusion includes a neck portion that connects the round portion to the body of the flexible PCB, the round protrusion configured to wrap over a side of the tissue engagement portion, anchor, or clasp and the one or more sutures are configured to extend over the neck portion at the side of the tissue engagement portion, anchor, or clasp to secure the flexible PCB to the tissue engagement portion, anchor, or clasp, and/or applying a force to the electrical lead causes the round protrusion to deform to allow the flexible PCB to slide from under the suture thereby releasing the flexible PCB from the tissue engagement portion, anchor, or clasp and leaving the one or more sutures coupled to the tissue engagement portion, anchor, or clasp.

Example 58: The system, apparatus, and/or device of example 54, wherein: the one or more physical features comprise a pair of side indents formed from the body of the flexible PCB, the side indents formed in opposite sides of the body of the flexible PCB, the side indents are configured to provide a positive lock in a targeted location of the flexible PCB, the targeted location configured so as to not interfere with measurements made with the one or more electrodes of the flexible PCB, and/or applying a force to the electrical lead causes the flexible PCB to slide from under the suture thereby releasing the flexible PCB from the tissue engagement portion, anchor, or clasp and leaving the one or more sutures coupled to the tissue engagement portion, anchor, or clasp.

Example 59: The system, apparatus, and/or device of example 54, wherein: the one or more physical features comprise a hole formed in the body of the flexible PCB near an edge of the body of the flexible PCB opposite an end from which the electrical lead extends away from the body of the flexible PCB, the one or more sutures are configured to pass through the hole over the body of the flexible PCB to secure the flexible PCB to the tissue engagement portion, anchor, or clasp, and/or applying a force to the electrical lead causes the body of the PCB to break at the edge of the body of the flexible PCB thereby releasing the flexible PCB from the tissue engagement portion, anchor, or clasp and leaving the one or more sutures coupled to the tissue engagement portion, anchor, or clasp.

Example 60: The system, apparatus, and/or device of example 59, wherein the one or more physical features further includes a relief extending from the hole to the edge, the relief configured to allow the one or more sutures to pass through the relief to release the flexible PCB from the tissue engagement portion, anchor, or clasp.

Example 61: The system, apparatus, and/or device of example 54, wherein: the one or more physical features comprise a pair of bi-directional tongues that form a pair of tabs on the body of the flexible PCB, the pair of tabs being oriented in opposite directions from one another, each of the pair of tabs being is configured to allow a suture of the one or more sutures to pass over a portion of the body of the flexible PCB and under the tabs to secure the flexible PCB to the tissue engagement portion, anchor, or clasp, and/or applying a force to the electrical lead causes the one or more sutures to push the corresponding tab away from the body of the PCB to allow the flexible PCB to slide from under the one or more sutures thereby releasing the flexible PCB from the tissue engagement portion, anchor, or clasp and leaving the one or more sutures coupled to the tissue engagement portion, anchor, or clasp.

Example 62: A system, an apparatus, and/or device usable for repairing a native valve, the system, apparatus, and/or device comprising: a tissue engagement portion, anchor, or clasp comprising a first arm and a second arm (optionally “surface” can be used in place of “arm”, e.g., “first surface” in place of “first arm” and “second surface” in place of “second arm” in any of examples 62-80) configured such that the first arm and the second arm can close or be moved closer together to capture tissue (e.g., a leaflet of the native valve) in the tissue engagement portion, anchor, or clasp, at least one of the first arm and the second arm being movable to form a capture region therebetween for capturing the tissue (e.g., a leaflet of the native valve), the tissue engagement portion, anchor, or clasp further comprising a plurality of barbs to secure the tissue (e.g., a leaflet of the native valve) within the tissue engagement portion, anchor, or clasp; and/or a flexible printed circuit board (PCB) comprising an electrode pad or electrode array with one or more electrodes coupled to the electrode pad/array, and/or an electrical lead extending away from the electrode pad/array, wherein the system, apparatus, and/or device is configured such that: an electrical signal can be applied to the one or more electrodes through the electrical lead of the flexible PCB, a bioimpedance signal can be measured using the electrical lead based on or in response to the applied electrical signal, and/or application of a force to the electrical lead causes the flexible PCB to be removed from the system, apparatus, and/or device.

Example 63: The system, apparatus, and/or device of example 62, wherein the flexible PCB is configured to be pulled through a pair of barbs of the plurality barbs to remove the flexible PCB from the system, apparatus, and/or device.

Example 64: The system, apparatus, and/or device of example 63, wherein the electrical lead extends between the pair of barbs.

Example 65: The system, apparatus, and/or device of example 64, wherein the electrode pad/array of the flexible PCB has a width that is greater than a distance between the pair of barbs, the electrode pad/array of the flexible PCB configured to bend to fit between the pair of barbs.

Example 66: The system, apparatus, and/or device of example 65, wherein the width of the electrode pad/array is less than or equal to 1.875 times the distance between the pair of barbs.

Example 67: The system, apparatus, and/or device of example 65, wherein the width of the electrode pad/array is less than or equal to 1.25 times the distance between the pair of barbs.

Example 68: The system, apparatus, and/or device of any of examples 65-67, wherein the distance between the pair of barbs is less than or equal to 8 mm.

Example 69: The system, apparatus, and/or device of any of examples 65-67, wherein a force required to pull the electrode pad/array through the pair of barbs is less than or equal to 1.5 N.

Example 70: The system, apparatus, and/or device of example 62, wherein the flexible PCB is configured to be pulled around a side of the plurality barbs to remove the flexible PCB from the system, apparatus, and/or device.

Example 71: The system, apparatus, and/or device of example 70, wherein the electrical lead has a diagonal bend section leading immediately away from the electrode pad/array such that the electrode pad/array is laterally offset from the electrical lead so that the electrical lead lies along the side of the plurality of barbs while the electrode pad/array is within the tissue engagement portion, anchor, or clasp.

Example 72: The system, apparatus, and/or device of example 71, wherein pulling on the electrical lead causes the electrode pad/array to exit the tissue engagement portion, anchor, or clasp from a side of the tissue engagement portion, anchor, or clasp around the plurality of barbs.

Example 73: The system, apparatus, and/or device of example 72, wherein pulling on the electrical lead causes the diagonal bend section to contact the plurality of barbs so as to cause the electrode pad/array to move laterally relative to the plurality of barbs to exit the side of the tissue engagement portion, anchor, or clasp, using one or more barbs of the plurality of barbs as a fulcrum.

Example 74: The system, apparatus, and/or device of example 62, wherein the electrode pad/array includes a relief cut through the electrode pad/array such that application of a sufficient force causes the electrode pad/array to split apart into a first lateral portion and a second lateral portion.

Example 75: The system, apparatus, and/or device of example 74, wherein the flexible PCB further includes a second electrical lead, the electrical lead coupled to the first lateral portion of the electrode pad/array and the second electrical lead coupled to the second lateral portion of the electrode pad/array.

Example 76: The system, apparatus, and/or device of example 75, wherein the electrical lead and the second electrical lead each include diagonal bend sections in opposite directions so that the electrical lead and the second electrical lead are each laterally offset from the respective lateral portion of the electrode pad/array so that the electrical lead lies along a first side of the plurality of barbs and the second electrical lead lies along a second side of the plurality of barbs opposite the first side while the electrode pad/array is within the tissue engagement portion, anchor, or clasp.

Example 77: The system, apparatus, and/or device of example 76, wherein application of a proximal force on the electrical lead and the second electrical lead causes the electrode pad/array to split into the first lateral portion and the second lateral portion.

Example 78: The system, apparatus, and/or device of example 77, wherein application of the proximal force on the electrical lead and the second electrical lead after the electrode split in the first lateral portion and the second lateral portion causes the first lateral portion to exit the tissue engagement portion, anchor, or clasp around the first side of the plurality of barbs and causes the second lateral portion to exit the tissue engagement portion, anchor, or clasp around the second side of the plurality of barbs.

Example 79: The system, apparatus, and/or device of any of examples 62-78, wherein the flexible PCB further includes a reference electrode coupled to the electrical lead.

Example 80: The system, apparatus, and/or device of any of examples 62-79, wherein the electrical lead is configured to extend proximally to a proximal end of a delivery system configured to implant the system, apparatus, and/or device.

Example 81: A system, an apparatus, and/or a device usable for repairing a native valve, the system, apparatus, and/or device comprising: an anchor portion comprising a tissue engagement portion, anchor, or clasp having a first arm and a second arm (optionally “surface” can be used in place of “arm”, e.g., “first surface” in place of “first arm” and “second surface” in place of “second arm” in any of examples 81-86) configured to capture the tissue (e.g., a leaflet of the native valve); a distal portion configured to engage with an actuation element of a delivery system, the actuation element configured to rotate to deploy the anchor portion; an electrode coupled to the tissue engagement portion, anchor, or clasp; and/or one or more wires coupled to the electrode and coupled to the actuation element of the delivery system, wherein the system, apparatus, and/or device is configured such that: an electrical signal can be applied to the electrode through the one or more wires, a bioimpedance signal can be measured based on or in response to the applied electrical signal, and/or rotation of the actuation element of the delivery system causes the one or more wires to spool around the actuation element so as to pull the electrode off of the tissue engagement portion, anchor, or clasp to remove the electrode from the system, apparatus, and/or device.

Example 82: The system, apparatus, and/or device of example 81, wherein the one or more wires are secured to a collar that is affixed to the actuation element such that rotation of the actuation element causes the collar to rotate.

Example 83: The system, apparatus, and/or device of example 82, wherein one or more electrical leads are coupled to the one or more wires at the collar to provide electrical connectivity to a proximal end of the delivery system.

Example 84: The system, apparatus, and/or device of any of examples 81-83, wherein the electrode comprises a flexible printed circuit board.

Example 85: The system, apparatus, and/or device of any of examples 81-84, wherein the electrode is releasably secured to the tissue engagement portion, anchor, or clasp.

Example 86: The system, apparatus, and/or device of any of examples 81-85, wherein rotation of the actuation element further causes the electrode to spool around the actuation element, thereby removing the electrode and the one or more wires from the system, apparatus, and/or device.

Example 87: A system for repairing a native valve, the system comprising: a delivery system comprising: a catheter with a proximal end and a distal end; an actuation element; a wire extending within a lumen of the catheter from the proximal end of the catheter to the distal end of the catheter; and a capture mechanism at a distal end of the delivery system; and a treatment device comprising: an attachment portion comprising a proximal component (e.g., a collar, ring, extension, etc.) configured to engage with the capture mechanism of the delivery system; an anchor portion comprising a tissue engagement portion, anchor, or clasp having a first arm and a second arm (optionally “surface” can be used in place of “arm”, e.g., “first surface” in place of “first arm” and “second surface” in place of “second arm” in any of examples 87-120) configured to capture the tissue (e.g., a leaflet of the native valve); a distal portion configured to engage with the actuation element of the delivery system, the actuation element configured to deploy the anchor portion and to release the capture mechanism from the proximal component; an electrode coupled to the tissue engagement portion, anchor, or clasp; and/or an electrical lead having a distal end coupled to the electrode and a proximal end coupled to the proximal component, wherein the treatment device is configured such that: an electrical signal can be applied to the electrode through the electrical lead, and/or a bioimpedance signal can be measured based on or in response to the applied electrical signal, and/or wherein the wire is configured to provide an electrical connection to the electrical lead during delivery and deployment of the treatment device that is terminated upon withdrawal of the delivery system.

Example 88: The system of example 87, wherein: a distal end of the wire comprises a spring pin connector, the proximal end of the electrical lead is coupled to an electrical pad at the proximal component, and/or the spring pin connector of the wire is in electrical contact with the electrical pad of the electrical lead to provide electrical connection to the electrode until the treatment device is released from the delivery system.

Example 89: The system of example 87, wherein: a distal end of the wire comprises an electrical pad, the proximal end of the electrical lead is coupled to a spring pin connector at the proximal component, and/or the spring pin connector of the electrical lead is in electrical contact with the electrical pad of the wire to provide electrical connection to the electrode until the treatment device is released from the delivery system.

Example 90: The system of any of examples 87-89, wherein the spring pin connector is configured to use spring forces parallel to a shaft of the catheter to provide electrical contact between the electrical lead and the wire.

Example 91: The system of any of examples 87-90, wherein a spring force of the spring pin connector is configured to assist in detaching the spring pin connector from the electrical pad.

Example 92: The system of example 87, wherein: the proximal component forms a groove, the electrical lead is coupled to the proximal component within the groove; the capture mechanism comprises a finger configured to mate with the groove of the proximal component to couple the treatment device to the delivery system, the wire is coupled to an inner surface of the finger so that the wire physically contacts the electrical lead in the groove to provide electrical contact between the wire and the electrical lead, and/or release of the treatment device from the delivery system causes the finger to disengage from the proximal component, thereby releasing the treatment device and terminating electrical contact between the wire and the electrical lead.

Example 93: The system of example 92, wherein the groove and the finger are coated with an insulative material to electrically isolate the electrical connection between the wire and the electrical lead.

Example 94: The system of example 87, wherein: the delivery system further comprises a tube coupled to the capture mechanism with the wire secured within the tube, the proximal end of the electrical lead is releasably secured within the tube to provide electrical contact between the wire and the electrical lead while the treatment device is coupled to the delivery system, and withdrawal of the delivery system from the treatment device causes the tube to move away from the proximal component, thereby releasing the electrical lead from the tube and terminating electrical contact between the wire and the electrical lead.

Example 95: The system of example 94, wherein the tube comprises a leaf spring to provide a clamping force on the wire and the electrical lead to enhance the electrical connection.

Example 96: The system of any of examples 94-95, wherein the delivery system further comprises a frame secured to the distal end of the catheter, the tube being coupled to the frame and the frame configured to hold the tube in a targeted location relative to the treatment device.

Example 97: The system of example 96, wherein the frame is made of a polymer to electrically isolate the electrical connection between the wire and the electrical lead.

Example 98: The system of any of examples 96-97, wherein the frame comprises a U-shaped support that engages with the attachment portion of the treatment device.

Example 99: The system of example 87, wherein: a distal end of the wire terminates with a coil crimp having an inner diameter, the proximal end of the electrical lead is seated within the coil crimp, the inner diameter configured to provide a friction fit between the electrical lead and the wire to establish an electrical connection between the wire and the electrical lead, and the coil crimp is configured to expand to release the electrical lead.

Example 100: The system of example 99, wherein the coil crimp is configured to expand responsive to being exposed to a temperature above a threshold temperature.

Example 101: The system of example 99, wherein the coil crimp is configured to expand responsive to a current above a threshold current being driven through the wire.

Example 102: The system of any of examples 99-101, wherein the coil crimp is formed with a shape memory alloy in a martensite state, the inner diameter being smaller than a diameter of the electrical lead.

Example 103: The system of example 102, wherein the coil crimp is configured to expand to have an inner diameter larger than the diameter of the electrical lead responsive to transitioning to the austenite state.

Example 104: The system of any of examples 99-103, wherein the coil crimp includes a bent location to enhance a friction fit between the wire and the electrical lead.

Example 105: The system of example 104, wherein the electrical lead is inserted into the coil crimp at the bent location.

Example 106: The system of example 87, wherein: the capture mechanism further comprises a pair of fingers that are configured to engage with the proximal component to releasably secure the treatment device to the delivery system, the capture mechanism further comprises a disc crimp having a first section coupled to a first finger of the pair of fingers and a second section coupled to a second finger of the pair of fingers, the first section and the second section of the disc crimp forming a connection channel when abutted by the pair of fingers, the connection channel opening when the first section and the second section are separated, the wire is coupled to the connection channel and the electrical lead is seated within the connection channel, the connection channel is sized to force the wire to physically contact the electrical lead to form an electrical connection, releasing the treatment device from the delivery system causes the pair of fingers to separate from the proximal component and to separate the first section from the second section of the disc crimp, thereby allowing the wire and the electrical lead to separate to terminate the electrical connection.

Example 107: The system of example 106, wherein the disc crimp comprises a polymer that is configured to electrically insulate the electrical connection between the wire and the electrical lead.

Example 108: The system of any of examples 106-107, wherein: the first section is coupled to the first finger by inserting a portion of the first section through a window of the first finger to establish a friction fit between the first section and the first finger, and the second section is coupled to the second finger by inserting a portion of the second section through a window of the second finger to establish a friction fit between the second section and the second finger.

Example 109: The system of any of examples 106-108, wherein the first section and the second section comprise a shape set alloy that is welded to the first finger and the second finger, respectively.

Example 110: The system of example 109, wherein the connection channel is coated with an electrically insulative coating to electrically insulate the electrical connection between the wire and the electrical lead.

Example 111: The system of example 87, wherein: the delivery system further comprises a heat-activated electrical connector coupled to the capture mechanism with the wire secured within the heat-activated electrical connector, the proximal end of the electrical lead is releasably secured within the heat-activated electrical connector to provide electrical contact between the wire and the electrical lead while the treatment device is coupled to the delivery system, the heat-activated electrical connector is configured to change shape responsive to the application of heat or current, the change in shape configured to release the electrical lead from the heat-activated electrical connector, and/or withdrawal of the delivery system from the treatment device includes applying heat or current to the heat-activated electrical connector to cause the heat-activated electrical connector to open to release the electrical lead, thereby releasing the electrical lead from the heat-activated electrical connector and terminating electrical contact between the wire and the electrical lead.

Example 112: The system of example 111, wherein the heat-activated electrical connector comprises a shape set alloy with a transition temperature above average body temperature.

Example 113: The system of any of examples 111-112, wherein the heat-activated electrical connector is heated using heated saline.

Example 114: The system of any of examples 111-112, wherein the heat-activated electrical connector is opened by applying a current via the wire.

Example 115: The system of any of examples 111-114, wherein the heat-activated electrical connector comprises a flat tube with an open orifice that is configured to transition to an open U-shape responsive to the application of heat or current above a threshold to enable removal of the electrical lead.

Example 116: The system of any of examples 111-114, wherein the heat-activated electrical connector comprises a flat tube that is configured to transition to an open cylinder responsive to the application of heat or current above a threshold to enable removal of the electrical lead.

Example 117: The system of example 87, wherein: a distal end of the wire comprises a shape memory alloy that is formed into a shepherd hook and configured to transition to a straight wire with application of heat or current, a proximal end of the electrical lead comprises a shape memory alloy that is formed into a shepherd hook and configured to transition to a straight wire with application of heat or current, the shepherd hook of the wire and the shepherd hook of the electrical lead are hooked to each other to form an electrical connection, and/or withdrawal of the delivery system from the treatment device includes applying heat or current to the distal end of the wire and to the proximal end of the electrical lead to cause the wire and the electrical lead straighten, thereby disconnecting the electrical lead and the wire to terminate the electrical connection between the wire and the electrical lead.

Example 118: The system of example 117, wherein the heat-activated electrical connector is heated using heated saline.

Example 119: The system of example 117, wherein the heat-activated electrical connector is opened by applying a current via the wire.

Example 120: The system of any of examples 117-119, wherein: the wire comprises a first portion comprising a first metal and a second portion comprising the shape memory alloy, the first portion joined to the second portion using a first crimp, and/or the electrical lead comprises a first portion comprising the first metal and a second portion comprising the shape memory alloy, the first portion joined to the second portion using a second crimp.

Example 121: A device, the device comprising: (A) a tissue engagement portion or tissue capture portion comprising a first surface and a second surface, the tissue capture portion configured such that the first surface and the second surface can close or be moved closer together to capture tissue in the tissue capture portion, at least one of the first surface and the second surface being movable to form a capture region between the first surface and the second surface for capturing the tissue; and/or (B) two or more electrodes coupled to the tissue capture portion, and/or wherein the device is configured such that: (i) an electrical signal can be applied to the two or more electrodes, and/or (ii) a bioimpedance signal can be measured responsive to the electrical signal applied, the bioimpedance signal providing an indication of a tissue capture status within the tissue capture portion.

Example 122: The device of example 121, wherein the two or more electrodes comprise a first electrode coupled to the first surface and a second electrode coupled to the second surface.

Example 123: The device of example 122, wherein the first electrode is adjacent to the second electrode when the tissue capture portion is in a closed configuration.

Example 124: The device of example 123, wherein the first electrode comprises an electrode plate covering a majority of the first surface and the second electrode comprises an electrode plate covering a majority of the second surface.

Example 125: The device of any of examples 121-124, wherein the two or more electrodes comprise a first electrode coupled to the first surface and a second electrode coupled to the first surface.

Example 126: The device of example 125, wherein the first electrode is separated from the second electrode by a gap.

Example 127: The device of example 126, wherein the first electrode and the second electrode comprise electrode strips parallel to a length of the first surface.

Example 128: The device of example 126, wherein the first electrode and the second electrode comprise electrode strips parallel to a width of the first surface.

Example 129: The device of example 128, wherein the first electrode is positioned on the first surface at a first tissue capture depth.

Example 130: The device of example 129, wherein the second electrode is positioned on the first surface at a second tissue capture depth greater than the first tissue capture depth.

Example 131: The device of example 126 further comprising an electrode plate coupled to the second surface.

Example 132: The device of any of examples 121-131 further comprising an impedance measurement device configured to measure bioimpedance signals and/or to determine a tissue capture depth based on or in response to the measured bioimpedance signals.

Example 133: The device of example 132, wherein the impedance measurement device implements an algorithm to generate an indicator of fully captured tissue, partially captured tissue, or overly captured tissue.

Example 134: The device of example 132, wherein the impedance measurement device implements an algorithm to generate an indicator of tissue capture depth.

Example 135: The device of any of examples 132-134, wherein the impedance measurement device is configured to generate an indicator of tissue capture status when the tissue capture portion is in a closed configuration.

Example 136: The device of any of examples 132-135, wherein the impedance measurement device is configured to generate an indicator of tissue capture status when the tissue capture portion is in an open configuration.

Example 137: An implantable device configured to be implanted during a medical procedure that includes an anchor configured to secure the implantable device to tissue in a patient; and/or at least one electrode coupled to the anchor, wherein an electrical signal can be applied to the anchor, and a bioimpedance signal can be measured based on or in response to the applied electrical signal, the bioimpedance signal being indicative of a deployment status of the anchor.

Example 138: The implantable device of any example herein, in particular example 137, wherein the implantable device comprises an annuloplasty device.

Example 139: The implantable device of any example herein, in particular examples 137-138 further comprising a plurality of anchors, each anchor including at least one electrode.

Example 140: The implantable device of any example herein, in particular example 139, wherein an electrical signal can be applied to the plurality of anchors and a bioimpedance signal can be measured from each of the plurality of anchors based on or in response to the applied electrical signal, each bioimpedance signal configured to indicate a deployment status of the corresponding anchor of the plurality of anchors.

Example 141: The implantable device of any example herein, in particular examples 137-138 further comprising a plurality of anchors that are electrically shorted together.

Example 142: The implantable device of any example herein, in particular examples 137-141 further comprising an impedance measurement device configured to measure bioimpedance signals and/or to determine an anchor deployment status based on or in response to the measured bioimpedance signals.

Example 143: The implantable device of any example herein, in particular example 142, wherein the impedance measurement device implements an algorithm to generate an indicator of anchor deployment status, the anchor deployment status including the anchor in contact with tissue, a partially deployed anchor, and/or a fully deployed anchor.

Example 144: A system, an apparatus, and/or a device usable for repairing a native valve of a patient includes a tissue engagement portion, anchor, or clasp comprising a first arm and a second arm (optionally “surface” can be used in place of “arm”, e.g., “first surface” in place of “first arm” and “second surface” in place of “second arm” in any of examples 144-159) joined by a hinge portion to enable the first arm and the second arm to close or be moved closer together to capture targeted tissue in the tissue engagement portion, anchor, or clasp, the tissue engagement portion, anchor, or clasp being movable to form a capture region for capturing tissue (e.g., a leaflet of the native valve); and/or two or more electrodes coupled to the tissue engagement portion, anchor, or clasp, wherein an electrical signal can be applied to the two or more electrodes, and/or a bioimpedance signal can be measured based on or in response to the electrical signal applied, the bioimpedance signal configured to indicate a leaflet capture status within the tissue engagement portion, anchor, or clasp.

Example 145: The system, apparatus, and/or device of any example herein, in particular example 144, wherein the two or more electrodes comprise a first electrode coupled to the first arm and a second electrode coupled to the second arm.

Example 146: The system, apparatus, and/or device of any example herein, in particular example 145, wherein the first electrode is adjacent to the second electrode upon closing the tissue engagement portion, anchor, or clasp.

Example 147: The system, apparatus, and/or device of any example herein, in particular example 146, wherein the first electrode comprises an electrode plate covering a majority of the first arm and the second electrode comprises an electrode plate covering a majority of the second arm.

Example 148: The system, apparatus, and/or device of any example herein, in particular examples 144-147, wherein the two or more electrodes comprise a first electrode coupled to the first arm and a second electrode coupled to the first arm.

Example 149: The system, apparatus, and/or device of any example herein, in particular example 148, wherein the first electrode is separated from the second electrode by a gap.

Example 150: The system, apparatus, and/or device of any example herein, in particular example 149, wherein the first electrode and the second electrode comprise electrode strips parallel to a length of the first arm.

Example 151: The system, apparatus, and/or device of any example herein, in particular example 149, wherein the first electrode and the second electrode comprise electrode strips parallel to a width of the first arm.

Example 152: The system, apparatus, and/or device of any example herein, in particular example 151, wherein the first electrode is positioned on the first arm at a targeted minimum leaflet capture depth.

Example 153: The system, apparatus, and/or device of any example herein, in particular example 152, wherein the second electrode is positioned on the first arm at a targeted maximum leaflet capture depth.

Example 154: The system, apparatus, and/or device of any example herein, in particular example 149 further comprising an electrode plate coupled to the second arm.

Example 155: The system, apparatus, and/or device of any example herein, in particular examples 144-154 further comprising an impedance measurement device configured to measure bioimpedance signals and to determine a leaflet capture depth based on the measured bioimpedance signals.

Example 156: The system, apparatus, and/or device of any example herein, in particular example 155, wherein the impedance measurement device is configured to implement an algorithm to generate an indicator of a fully captured leaflet, a partially captured leaflet, or an overly captured leaflet.

Example 157: The system, apparatus, and/or device of any example herein, in particular example 155, wherein the impedance measurement device is configured to implement an algorithm to generate an indicator of leaflet capture depth.

Example 158: The system, apparatus, and/or device of any example herein, in particular examples 155-157, wherein the impedance measurement device is configured to generate an indicator of leaflet capture status when the tissue engagement portion, anchor, or clasp is closed.

Example 159: The system, apparatus, and/or device of any example herein, in particular examples 155-158, wherein the impedance measurement device is configured to generate an indicator of leaflet capture status when the tissue engagement portion, anchor, or clasp is open.

Example 160: A bioimpedance signal measurement system and/or apparatus comprising: a device including a tissue engagement portion comprising a first surface and a second surface, the tissue engagement portion configured such that the first surface and the second surface can close or be moved closer together to capture tissue in the tissue engagement portion, at least one of the first surface and the second surface being movable to form a capture region between the first surface and the second surface for capturing the tissue; and/or two or more electrodes coupled to the tissue engagement portion; and/or an impedance measurement device comprising a power supply and an electrical sensor, the power supply configured to apply an electrical signal to the two or more electrodes, the impedance measurement device configured to measure a bioimpedance signal using the electrical sensor, the bioimpedance signal responsive to the applied electrical signal, the bioimpedance signal providing an indication of a status of the tissue within the tissue engagement portion.

Example 161: The system, apparatus, and/or device of any example herein, in particular example 160, wherein the two or more electrodes are coupled to one or more anchors of the device.

Example 162: The system, apparatus, and/or device of any example herein, in particular example 160, wherein the two or more electrodes are coupled to one or more clasps of the device.

Example 163, The system, apparatus, and/or device of any example herein, in particular examples 160-162, wherein the impedance measurement device is configured to measure electrical characteristics from the two or more electrodes to determine the relative location of a clasp of the device and anatomy that the device is in contact with.

Example 164: The system, apparatus, and/or device of any example herein, in particular example 163, wherein the electrical characteristics includes a peak-to-peak amplitude of oscillations of the bioimpedance signal.

Example 165: The system, apparatus, and/or device of any example herein, in particular example 163, wherein the electrical characteristics includes an average value of a magnitude of the bioimpedance signal.

Example 166: The system, apparatus, and/or device of any example herein, in particular examples 160-165, wherein the system is further configured to determine that the two or more electrodes are in blood based at least in part on the bioimpedance signal.

Example 167: The system, apparatus, and/or device of any example herein, in particular examples 160-166, wherein the system is further configured to determine that the two or more electrodes are contacting targeted tissue based at least in part on the bioimpedance signal.

Example 168: The system, apparatus, and/or device of any example herein, in particular examples 160-167, wherein the system is further configured to differentiate tissue types based at least in part on the bioimpedance signal.

Example 169: The system, apparatus, and/or device of any example herein, in particular examples 160-168, wherein the system is further configured to determine that the two or more electrodes are transitioning from being primarily in contact with blood to being partially in contact with tissue based at least in part on the bioimpedance signal.

Example 170: The system, apparatus, and/or device of any example herein, in particular examples 160-169, wherein the system is further configured to determine that the two or more electrodes are transitioning from being partially or primarily in contact with tissue to being primarily in contact with blood based at least in part on the bioimpedance signal.

Example 171: The system, apparatus, and/or device of any example herein, in particular examples 160-170, wherein the impedance measurement device implements a signal processing algorithm to indicate a status of the device.

Example 172: The system, apparatus, and/or device of any example herein, in particular example 171, wherein the status of the device includes full capture of a leaflet, under capture of a leaflet, over capture of a leaflet, and a relative position of a leaflet in a clasp of the device.

Example 173: The system, apparatus, and/or device of any example herein, in particular examples 160-172 further comprising a display to display a derived indicator to a user, the derived indicator indicative of a status of the device.

Example 174: A system, apparatus, and/or device for use in medical procedures, the system, apparatus, and/or device comprising: (A) a tissue engagement portion comprising a first surface and a second surface configured such that the first surface and the second surface can close or be moved closer together to capture a tissue in the tissue engagement portion, at least one of the first surface and the second surface being movable to form a capture region therebetween for capturing the tissue, the tissue engagement portion further comprising one or more friction-enhancing elements to secure the tissue within the tissue engagement portion; (B) one or more electrodes, and/or (C) an electrical lead electrically coupled to the one or more electrodes.

Example 175: The system, apparatus, and/or device of example 174, wherein the system, apparatus, and/or device is configured such that: (i) an electrical signal can be applied to the one or more electrodes through the electrical lead, (ii) a bioimpedance signal can be measured using the electrical lead based on or in response to the applied electrical signal, and/or (iii) application of a force to the electrical lead causes the one or more electrodes to be removed from the tissue engagement portion.

Example 176: The system, apparatus, and/or device of any one of examples 174-175, wherein the one or more electrodes are configured to be pulled through a pair of barbs of the one or more friction-enhancing elements to remove the one or more electrodes from the tissue engagement portion.

Example 177: The system, apparatus, and/or device of example 176, wherein the electrical lead extends between the pair of barbs.

Example 178: The system, apparatus, and/or device of example 177, wherein the one or more electrodes are configured to bend to fit between the pair of barbs.

Example 179: The system, apparatus, and/or device of any one of examples 174-175, wherein the one or more electrodes are configured to be pulled around a side of the one or more friction-enhancing elements to remove the one or more electrodes from the tissue engagement portion.

Example 180: The system, apparatus, and/or device of example 179, wherein the electrical lead has a diagonal bend section leading immediately away from the one or more electrodes such that the lead is laterally offset and lies along the side of the one or more friction-enhancing elements while the one or more electrodes are within the tissue engagement portion.

Example 181: The system, apparatus, and/or device of example 180, wherein pulling on the electrical lead causes the one or more electrodes to exit the tissue engagement portion from a side of the tissue engagement portion around the one or more friction-enhancing elements.

Example 182: The system, apparatus, and/or device of example 181, wherein pulling on the electrical lead causes the diagonal bend section to contact the one or more friction-enhancing elements so as to cause the one or more electrodes to move laterally relative to the plurality of barbs to exit the side of the tissue engagement portion, using one or more of the one or more friction-enhancing elements as a fulcrum.

Example 183: The system, apparatus, and/or device of any one of examples 174-175, wherein the one or more electrodes are mounted on an electrode pad that includes a relief cut through the electrode pad such that application of a sufficient force causes the electrode pad to split apart into a first lateral portion and a second lateral portion.

Example 184: The system, apparatus, and/or device of example 183, wherein the system, apparatus, and/or device further includes a second electrical lead, the electrical lead coupled to the first lateral portion of the one or more electrodes and the second electrical lead coupled to the second lateral portion of the one or more electrodes.

Example 185: The system, apparatus, and/or device of example 184, wherein the electrical lead and the second electrical lead each include diagonal bend sections in opposite directions so that the electrical lead and the second electrical lead are each laterally offset so that the electrical lead lies along a first side of the one or more friction-enhancing elements and the second electrical lead lies along a second side of the one or more friction-enhancing elements opposite the first side while the one or more electrodes are within the tissue engagement portion.

Example 186: The system, apparatus, and/or device of example 185, wherein application of a proximal force on the electrical lead and the second electrical lead causes the electrode pad to split into the first lateral portion and the second lateral portion.

Example 187: The system, apparatus, and/or device of example 186, wherein application of the proximal force on the electrical lead and the second electrical lead causes the first lateral portion to exit the tissue engagement portion around the first side of the one or more friction-enhancing elements and causes the second lateral portion to exit the tissue engagement portion around the second side of the one or more friction-enhancing elements.

Example 188: The system, apparatus, and/or device of examples 174-187, wherein the system, apparatus, and/or device further includes a reference electrode coupled to the electrical lead.

Example 189: The system, apparatus, and/or device of examples 174-188, wherein the system, apparatus, and/or device is a valve repair device and is configured to capture leaflet tissue of a native valve in the tissue engagement portion.

Example 190: The system, apparatus, and/or device of any example herein, wherein the system, apparatus, and/or device is sterilized.

Example 191: A method comprising sterilizing any of the systems, apparatuses, and/or devices of examples 1-190.

Example 192: A system, apparatus, and/or device (e.g., a feedback system, indication system, bioimpedance-based feedback system, etc.) comprising: a data store configured to store computer executable instructions; and a processor connected to the data store and configured to execute the stored computer executable instructions to cause the processor to: measure a bioimpedance signal that is responsive to an electrical signal applied to a tissue engagement portion of an implantable device; determine a status of tissue relative to the tissue engagement portion (e.g., whether tissue has been captured in the tissue engagement portion, whether tissue is folded or bunched in the tissue engagement portion, whether the tissue engagement portion has penetrated tissue, etc.) based on the measured bioimpedance signal; and/or generate an indicator of the status.

Example 193: A method for using bioimpedance-based feedback to monitor a status of a system, apparatus, and/or device and/or a status of targeted tissue (e.g., of living tissue, of cadaver tissue, of simulated tissue, etc.), the method comprising: applying an electrical signal to a tissue engagement portion of the system, apparatus, or device; measuring a bioimpedance signal that is responsive to the electrical signal applied to the tissue engagement portion; and/or determining a status of the system, apparatus, and/or device and/or a status of tissue relative to the tissue engagement portion (e.g., whether tissue has been captured in the tissue engagement portion, whether tissue is folded or bunched in the tissue engagement portion, whether the tissue engagement portion has penetrated tissue, etc.) based on the measured bioimpedance signal.

Example 194: A method for using bioimpedance-based feedback to monitor a status of a system, apparatus, and/or device and/or a status of targeted tissue (e.g., of living tissue, of cadaver tissue, of simulated tissue, etc.), the method comprising: advancing the a system, apparatus, and/or device (e.g., all or a portion thereof) to a desired location inside a body of a subject (e.g., of a living subject, of a simulation, etc.), applying an electrical signal to a tissue engagement portion of the system, apparatus, or device; measuring a bioimpedance signal that is responsive to the electrical signal applied to the tissue engagement portion; and/or determining a status of the system, apparatus, and/or device and/or a status of tissue relative to the tissue engagement portion (e.g., whether tissue has been captured in the tissue engagement portion, whether tissue is folded or bunched in the tissue engagement portion, whether the tissue engagement portion has penetrated tissue, etc.) based on the measured bioimpedance signal

Example 195: The method of any one of examples 193 and 194, further comprising generating an indicator of the status.

Example 196: The method of any one of examples 193-195, wherein the system, apparatus, and/or device comprises and implant, and the method further includes implanting the implant inside the body of the subject.

Example 197: The method of any one of examples 193-196, further comprising, responsive to the status, adjusting the system, apparatus, and/or device (e.g., adjusting the tissue engagement portion) inside the body of the subject and/or relative to the tissue, subsequently applying a second electrical signal to the tissue engagement portion, and measuring a second bioimpedance signal that is responsive to the second electrical signal applied to the tissue engagement portion; and/or determining a second status of the system, apparatus, and/or device and/or a second status of tissue relative to the tissue engagement portion (e.g., whether tissue has been captured in the tissue engagement portion, whether tissue is folded or bunched in the tissue engagement portion, whether the tissue engagement portion has penetrated tissue, etc.) based on the measured second bioimpedance signal.

Example 198: The method of example 197, further comprising, responsive to the second status, adjusting the system, apparatus, and/or device (e.g., adjusting the tissue engagement portion) inside the body of the subject and/or relative to the tissue, subsequently applying a third electrical signal to the tissue engagement portion, and measuring a third bioimpedance signal that is responsive to the third electrical signal applied to the tissue engagement portion; and/or determining a third status of the system, apparatus, and/or device and/or a third status of tissue relative to the tissue engagement portion (e.g., whether tissue has been captured in the tissue engagement portion, whether tissue is folded or bunched in the tissue engagement portion, whether the tissue engagement portion has penetrated tissue, etc.) based on the measured third bioimpedance signal.

Example 199: The method of any one of examples 193-198, further comprising removing the system, apparatus, and/or device from the body of the subject.

Any of the various systems, assemblies, devices, components, apparatuses, etc. in this disclosure, including those in the examples above, can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of).

The various inventive aspects, concepts and features of the disclosures may be described and illustrated herein as embodied in combination in the examples herein, these various aspects, concepts, and features may be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc. The words used in the claims have their full ordinary meanings and are not limited in any way by the description of the implementations in the specification.

What is claimed is:

1. A device comprising:
a tissue engagement portion comprising a first arm and a second arm configured such that the first arm and the second arm can close or be moved closer together to capture tissue in the tissue engagement portion, at least one of the first arm and the second arm being movable to form a capture region therebetween for capturing the tissue; and
two or more electrodes coupled to the tissue engagement portion,
wherein the device is configured such that:
an electrical signal can be applied to the two or more electrodes, and
a bioimpedance signal can be measured responsive to the electrical signal applied.

2. The device of claim 1, wherein the bioimpedance signal provides an indication of a status of the tissue within the tissue engagement portion.

3. The device of claim 2, wherein the status includes under insertion of tissue in the tissue engagement portion, full insertion of tissue in the tissue engagement portion, over insertion of tissue in the tissue engagement portion, angled insertion of tissue in the tissue engagement portion, or insertion of non-targeted tissue in the tissue engagement portion.

4. The device of claim 2, wherein the status includes insertion of tissue in the tissue engagement portion while the tissue engagement portion is in an open configuration comprising the first arm and the second arm being apart from each other.

5. The device of claim 2, wherein the indication of the status is configured to be used to generate a visual indicator for a user of the status.

6. The device of claim 5, wherein the visual indicator is configured to indicate one or more of no tissue insertion, under tissue insertion, full tissue insertion, and over tissue insertion.

7. The device of claim 1, wherein the two or more electrodes comprise:
a first electrode strip coupled to the first arm of the tissue engagement portion near a first edge of the first arm; and
a second electrode strip coupled to the first arm of the tissue engagement portion near a second edge of the first arm, the second edge opposite the first edge,
wherein the first electrode strip and the second electrode strip are parallel to each other and run along a length of the first arm.

8. The device of claim 7, wherein the first electrode strip and the second electrode strip are offset a prescribed distance from a free edge of the first arm of the tissue engagement portion.

9. A valve repair device for repairing a native valve, the valve repair device comprising:
a tissue engagement portion comprising a first arm and a second arm configured such that the first arm and the second arm can close or be moved closer together to capture a tissue (e.g., a leaflet of the native valve) in the tissue engagement portion, at least one of the first arm and the second arm being movable to form a capture region therebetween for capturing the tissue (e.g., a leaflet of the native valve), the tissue engagement portion further comprising a plurality of barbs to secure the tissue (e.g., a leaflet of the native valve) within the tissue engagement portion; and
a flexible printed circuit board (PCB) comprising an electrode pad with one or more electrodes coupled to the electrode pad, and an electrical lead extending away from the electrode pad,
wherein the valve repair device is configured such that:
an electrical signal can be applied to the one or more electrodes through the electrical lead of the flexible PCB,
a bioimpedance signal can be measured using the electrical lead based on or in response to the applied electrical signal, and
application of a force to the electrical lead causes the flexible PCB to be removed from the valve repair device.

10. The valve repair device of claim 9, wherein the flexible PCB is configured to be pulled through a pair of barbs of the plurality barbs to remove the flexible PCB from the valve repair device.

11. The valve repair device of claim 10, wherein the electrical lead extends between the pair of barbs.

12. The valve repair device of claim 11, wherein the electrode pad of the flexible PCB has a width that is greater than a distance between the pair of barbs, the electrode pad of the flexible PCB configured to bend to fit between the pair of barbs.

13. The valve repair device of claim 9, wherein the flexible PCB is configured to be pulled around a side of the plurality barbs to remove the flexible PCB from the valve repair device.

14. The valve repair device of claim 9, wherein the electrode pad includes a relief cut through the electrode pad such that application of a sufficient force causes the electrode pad to split apart into a first lateral portion and a second lateral portion.

15. The valve repair device of claim 14, wherein the flexible PCB further includes a second electrical lead, the electrical lead coupled to the first lateral portion of the electrode pad and the second electrical lead coupled to the second lateral portion of the electrode pad.

16. The valve repair device of claim 9, wherein the electrical lead is configured to extend proximally to a proximal end of a delivery system configured to implant the valve repair device.

17. A system for repairing a native valve, the system comprising:
a delivery system comprising:
a catheter with a proximal end and a distal end;
an actuation element;
a wire extending within a lumen of the catheter from the proximal end of the catheter to the distal end of the catheter; and
a capture mechanism at a distal end of the delivery system; and
a valve repair device comprising:
an attachment portion comprising a proximal component configured to engage with the capture mechanism of the delivery system;
an anchor portion comprising a tissue engagement portion having a first arm and a second arm configured to capture the tissue (e.g., a leaflet of the native valve);
a distal portion configured to engage with the actuation element of the delivery system, the actuation element configured to deploy the anchor portion and to release the capture mechanism from the proximal component;
an electrode coupled to the tissue engagement portion; and
an electrical lead having a distal end coupled to the electrode and a proximal end coupled to the proximal component,
wherein the valve repair device is configured such that:
an electrical signal can be applied to the electrode through the electrical lead, and
a bioimpedance signal can be measured based on or in response to the applied electrical signal, and
wherein the wire is configured to provide an electrical connection to the electrical lead during delivery and deployment of the valve repair device that is terminated upon withdrawal of the delivery system.

18. The system of claim 17, wherein:
a distal end of the wire comprises a spring pin connector,
the proximal end of the electrical lead is coupled to an electrical pad at the proximal component, and
the spring pin connector of the wire is in electrical contact with the electrical pad of the electrical lead to provide electrical connection to the electrode until the valve repair device is released from the delivery system.

19. The system of claim 17, wherein:
a distal end of the wire comprises an electrical pad,
the proximal end of the electrical lead is coupled to a spring pin connector at the proximal component, and
the spring pin connector of the electrical lead is in electrical contact with the electrical pad of the wire to provide electrical connection to the electrode until the valve repair device is released from the delivery system.

20. The system of claim 17, wherein the spring pin connector is configured to use spring forces parallel to a shaft of the catheter to provide electrical contact between the electrical lead and the wire.

21. The system of claim 17, wherein a spring force of the spring pin connector is configured to assist in detaching the spring pin connector from the electrical pad.

22. The system of claim 17, wherein:
the proximal component forms a groove,
the electrical lead is coupled to the proximal component within the groove;
the capture mechanism comprises a finger configured to mate with the groove of the proximal component to couple the valve repair device to the delivery system,
the wire is coupled to an inner surface of the finger so that the wire physically contacts the electrical lead in the groove to provide electrical contact between the wire and the electrical lead, and
release of the valve repair device from the delivery system causes the finger to disengage from the proximal component, thereby releasing the valve repair device and terminating electrical contact between the wire and the electrical lead.

23. The system of claim 22, wherein the groove and the finger are coated with an insulative material to electrically isolate the electrical connection between the wire and the electrical lead.

24. The system of claim 17, wherein:
the delivery system further comprises a tube coupled to the capture mechanism with the wire secured within the tube,
the proximal end of the electrical lead is releasably secured within the tube to provide electrical contact between the wire and the electrical lead while the valve repair device is coupled to the delivery system, and
withdrawal of the delivery system from the valve repair device causes the tube to move away from the proximal component, thereby releasing the electrical lead from the tube and terminating electrical contact between the wire and the electrical lead.

25. The system of claim 24, wherein the tube comprises a leaf spring to provide a clamping force on the wire and the electrical lead to enhance the electrical connection.

26. The system of claim 24, wherein the delivery system further comprises a frame secured to the distal end of the catheter, the tube being coupled to the frame and the frame configured to hold the tube in a targeted location relative to the valve repair device.

27. The system of claim 17, wherein:
a distal end of the wire terminates with a coil crimp having an inner diameter,
the proximal end of the electrical lead is seated within the coil crimp, the inner diameter configured to provide a friction fit between the electrical lead and the wire to establish an electrical connection between the wire and the electrical lead, and
the coil crimp is configured to expand to release the electrical lead.

28. The system of claim 27, wherein the coil crimp is configured to expand responsive to being exposed to a temperature above a threshold temperature or to a current above a threshold current being driven through the wire.

29. The system of claim 27, wherein the coil crimp is formed with a shape memory alloy in a martensite state, the inner diameter being smaller than a diameter of the electrical lead, and to expand to have an inner diameter larger than the diameter of the electrical lead responsive to transitioning to the austenite state.
